(19) United States
(12) Patent Application Publication
(10) Pub. No.: US 2009/0226896 A1
(43) Pub. Date: Sep. 10, 2009

(54) Two Component DNA Replicases with Modified Beta-Subunit Binding Motifs, and Uses Thereof

(75) Inventor: Lars-Erik Peters, Lafayette, CO (US)

Correspondence Address: King Spalding LLP, 4 Embarcadero Center, Suite 3500, San Francisco, CA, 94111, US

(73) Assignee: Qiagen North American Holdings, Inc., Valencia, CA (US)

(21) Appl. No.: 11/910,900

(22) PCT Filed: Jan. 3, 2006
(86) PCT No.: PCT/US06/00077

§ 371 Date: Dec. 10, 2008

(51) Int. Cl. C12Q 1/68; C12N 9/12; C12P 19/34; C12Q 1/48

(52) U.S. Cl. 435/6; 435/194; 435/91.1; 435/15

(57) ABSTRACT

The invention provides Pol III α mutants, and modified Pol III replicases comprising the same, and methods of using modified Pol III replicases for a variety of nucleic acid replication reactions.

### STATEMENT OF RELATEDNESS

This application claims the benefit of U.S. Provisional Patent Application Ser. No. 60/641,183 filed 3 Jan. 2005, which is expressly incorporated herein in its entirety by reference.

### FIELD

The invention relates to the enzymatic synthesis, digestion, replication, and modification of nucleic acid molecules. The invention further relates to bacterial DNA Polymerase III enzymes, and variants thereof engineered for desirable characteristics.

### BACKGROUND OF THE INVENTION

DNA polymerases are used in fundamental processes in molecular biology, including nucleic acid sequencing, nucleic acid labeling, nucleic acid quantification (Real Time PCR, NASBA), nucleic acid amplification (PCR, RDA, SDA), and reverse transcription of RNA into cDNA.

DNA polymerases have been isolated from a variety of biological sources and characterized as multifunctional enzymes which typically possess at least two different catalytic activities. For example, retroviral reverse transcriptases possess an RNA-dependent DNA polymerase activity and an RNase H-type endonuclease activity. Other DNA polymerases used in PCR and sequencing, such as the thermostable Taq and Tth DNA polymerase I, possess in their native form a 5′→3′ exonuclease activity and a DNA-dependent DNA polymerase activity. Another group of DNA polymerases, which includes E. coli DNA polymerase and the sequencing enzyme T7 DNA polymerase, have two different exonuclease activities (5′→3′ and 3′→5′) and a DNA-dependent DNA polymerase activity. The use of multifunctional DNA polymerases in analytical methods requires in most cases the suppression or elimination of unwanted non-polymerase activities. This can be done by protein engineering deleting either complete domains where those activities reside or altering conserved sequence motifs in active sites by mutagenesis. In both scenarios, knowledge about the location and structure of active sites determining the enzymatic activities is necessary.

DNA polymerase III holoenzyme (“Pol III”) was first purified and determined to be the principal replicase of the E. coli chromosome by Kornberg (Kornberg, A., 1982 Supplement to DNA Replication, Freeman Publications, San Francisco, pp 122-125), which is hereby incorporated by reference. The three functional components of the E. coli DNA Polymerase III can be assembled into one holoenzyme where they are all connected together. This holoenzyme is composed of 10 subunits (McHenry, et al., J. Bio Chem., 252:6478-6484 (1977) and Maki, et al., J. Biol. Chem., 263:6551-6559 (1988), which are hereby incorporated by reference).

The three functional components of Pol III are (i) the “core” (i.e. the polymerase), β (i.e., the clamp), and the γ-complex (i.e., the clamp loader). The τ subunit holds together two cores to form the Pol III′ subassembly, and it binds one γ-complex to form Pol III*. The τ subunit and the γ subunit are both encoded by dnaX. Tau is the full length product, while γ is approximately the N-terminal ⅔ of τ and is formed by a translational frame shift (Tsuchihashi et al., “Translational Frameshifting Generates the γ Subunit of DNA Polymerase III Holoenzyme,” Proc. Natl. Acad. Sci., USA., 87:2516-2520 (1990), which is hereby incorporated by reference).

Within the “core” are three subunits: the α subunit (encoded by dnaE) contains the DNA polymerase activity (Blanar, et al., Proc. Natl. Acad. Sci. USA, 81:4622-4626 (1984), which is hereby incorporated by reference); the ε subunit (encoded by dnaQ, mutD) is the proofreading 3′→5′ exonuclease (Scheuermann, et al., Proc. Natl. Acad. Sci. USA, 81:7747-7751 (1985) and DeFrancesco, et al., J. Biol. Chem., 259:5567-5573 (1984), which are hereby incorporated by reference), and the θ subunit (encoded by holE) stimulates ε (Studwell-Vaughan et al., “DNA Polymerase III Accessory Proteins V. theta encoded by holE*,” J. Biol. Chem., 268:11785-11791 (1993), which is hereby incorporated by reference). The α subunit forms a tight 1:1 complex with ε (Maki, et al., J. Biol. Chem., 260:12987-12992 (1985) which is hereby incorporated by reference, and θ forms a 1:1 complex with ε (Studwell-Vaughan et al., “DNA Polymerase III Accessory Proteins V. theta encoded by holE*,” J. Biol. Chem., 268:11785-11791 (1993), which is hereby incorporated by reference).

The E. coli three component polymerase is highly efficient and completely replicates a uniquely primed bacteriophage single-strand DNA (“ssDNA”) genome coated with the ssDNA binding protein (“SSB”), at a speed of at least 500 nucleotides per second at 30° C. without dissociating from a 5 kb circular DNA even once (Fay, et al., J. Biol. Chem., 256:976-983 (1981); O'Donnell, et al., J. Biol. Chem., 260:12884-12889 (1985); and Mok, et al., J. Biol. Chem., 262:16644-16654 (1987), which are hereby incorporated by reference).

In thermophilic bacteria, the organization of a minimal functional DNA Pol III holoenzyme is less complex. The polymerase core can function very efficiently without the ε and θ subunits. The clamp loader complex can assemble without the participation of ψ and χ subunits into a functional initiation complex (for example, see Bruck et al., J. Biol. Chem., 277:17334-17348, 2002; Bullard et al., J. Biol. Chem., 277:13401-13408, 2002).

In archae bacteria, the genome replicase holoenzymes are assembled from the same three functional components, clamp loader (RCF), processivity clamp (PCNA-Proliferating Cell Nuclear Antigen) and polymerase core, but their subunit organization is different and these subunits do not share any significant sequence homology to bacterial Pol III subunits.

Functional motifs, referred to herein as motifs A, B, and C, described below, have been identified in Pol I DNA polymerases.

“Motif A”, which is located in the palm domain of Pol I enzymes and forms the bottom of the dNTP binding pocket, is involved in dNTP binding and discrimination between deoxyribonucleotides and ribonucleotides. Motif A is also involved in the binding of primed template molecules. An invariant aspartic acid residue followed always by a large hydrophobic amino acid within the motif A complexes with a catalytically important Mg2+ cation. This Mg2+ cation activates the 3′-terminal hydroxyl group of a primer to attack the alpha phosphodiester bond of the incoming dNTP.

“Motif B”, which is located in the finger domain and forms the side and top of the dNTP binding pocket, is also involved in dNTP binding and discrimination between deoxyribonucleotides and ribonucleotides, as well as between deoxyribonucleotides and dideoxyribonucleotides. Motif B is also involved in primed template binding and interacts with the phosphate-sugar backbone of the three terminal bases of the primer. Conserved phenylalanine and tyrosine residues within motif B interact with the base moiety of the incoming dNTP by pi electron stacking interactions. An invariable lysine residue in the N-terminal half of motif B is engaged in electrostatic interactions with the gamma and beta orthophosphate groups of the incoming dNTP.

“Motif C”, which is isolated in the palm domain, forms the catalytic active site of the DNA polymerase. Two conserved aspartic acid residues (sometimes three) within motif C coordinate the second catalytically important Mg²⁺ cation that is complexed with the polymerase. This Mg²⁺ cation activates the alpha phosphodiester bond of the incoming dNTP.

Manipulation of these motifs has resulted in polymerases with altered nucleotide discrimination characteristics and altered template nucleic acid specificities.

The DNA Pol III polymerases have generally been thought to operate by distinct mechanisms (for example, see Steitz, J. Biol. Chem., 274:17395-17398, 1999; Mar Alba, Genome Biology, 2: reviews 3002.1-3002.4, 2001). However, Fijalkowska et al. previously reported the identification of putative conserved motifs in Pol III α through sequence alignment (Genetics 154:1039-1044, 1993), and Kim et al have reported the identification of conserved acidic residues putatively involved in divalent cation coordination at the Pol III α active site (J. Bacteriology 179:6721-6728, 1997).

In their report, Fijalkowska et al. identified putative motifs A, B, and C, arranged as A-B-C from the amino terminus to the carboxy terminus as in Pol I enzymes. However, no data was provided to support the function of the putative motifs suggested by sequence alignment, and the functional Pol III mutations examined by Fijalkowski et al. mapped outside their designated motifs A, B, and C.

Building off of active site descriptions for Pol I polymerases, Kim et al. identified by alignment two aspartate residues conserved in Pol IIIs and putatively involved in divalent cation coordination in the Pol III active site. Further, they identified five candidate positions for a third conserved acidic residue. However, the authors conceded that the data provided was insufficient to make definitive conclusions regarding the location of the Pol III active site.

### SUMMARY OF THE INVENTION

Disclosed herein is the identification of DNA polymerase functional motifs A, B, and C, and the unusual arrangement thereof in bacterial DNA Pol III α subunits. These motifs and their arrangement, including their spacings, are highly conserved in DNA Pol III α subunits, presumably owing to their critical function in catalysis, primer selectivity, and nucleotide discrimination. The arrangement of these motifs in the order C, A, B in the N- to C-terminus direction contradicts a previous report (Fijalkowska et al. supra) and is unique among all known polymerase classes and specific for bacterial genomic DNA replicases. Further, motifs and arrangements particular to gram negative bacteria DnaE, gram positive bacteria DnaE, gram positive bacteria PolC, and cyanobacteria DnaE are disclosed herein and may be used to distinguish between these different types of Pol III α subunits.

Stemming from this discovery, in one aspect, disclosed herein are sequence-based classification and activity determination methods. The classification and activity determination methods of the present invention are convenient sequence-based methods that provide information concerning the potential utility of previously uncharacterized and/or novel proteins in a number of applications, including nucleic acid molecule amplification and nucleic acid molecule sequencing. Further disclosed herein are compositions and methods for diagnosing a bacterial infection.

The consensus sequences for motifs A, B, and C of the active site of DnaE from gram negative bacteria are, respectively, G-[L/M]-[L/V/I]-K-X-D-F-L-G-L-X-X-L-T, [F/W]-X-X-X-X-X-F-X-X-Y-[A/G]-F-N-K-S-H, and S-X-P-D-[F/I]-D-X-D-[F/I], wherein X is any amino acid. The arrangement of the motifs, from N-terminus to C-terminus is C-A-B, with a spacing between motifs C-A of about 153-155 amino acids, a spacing between motifs A-B of about 195-201 amino acids, and a consequent spacing between motifs C-B of about 348-356 amino acids. This information provides for a sequence-based method of determining that a polypeptide is a Pol III α subunit from gram negative bacteria. The method involves determining the amino acid sequence of a candidate polypeptide, or a segment thereof, and identifying therein the amino acid sequence of gram negative consensus motifs A, B, and C, or C and A, or A and B, or C and B, with the arrangement characteristic of gram negative DnaE. In an alternative embodiment, the methods consist essentially of identifying in the amino acid sequence of the polypeptide, or portion thereof, the consensus gram negative DnaE motifs A, B, and C. In another embodiment, the methods consist essentially of identifying in the amino acid sequence of the polypeptide, or portion thereof, the consensus gram negative DnaE motifs A, B, or A and B, and optionally C. The sequence based methods may be combined with other activity assays.

Similarly, the consensus sequences for motifs A, B, and C of the active site of DnaE from cyanobacteria are, respectively, G-L-L-K-M-D-F-L-G-L-[R/K]-N-L-T, F-D-Q-M-V-K-F-A-E-Y-C-F-N-K-S-H, P-D-I-D-T-D-F-C. The motifs are arranged, from amino terminus to carboxyl terminus, in the order C-A-B. The spacing between motif C and A is about 100-160 amino acids. The spacing between motif A and motif B about 150-210 amino acids. The spacing from motif C to motif B is about 250-370 amino acids. This information provides for a sequence-based method of determining that a polypeptide is a Pol III α subunit from cyanobacteria. The method involves determining the amino acid sequence of a candidate polypeptide, or a segment thereof, and identifying therein the amino acid of cyanobacteria consensus motifs A, B, and C, or C and A, or A and B, or C and B, with the arrangement characteristic of cyanobacteria DnaE. In an alternative embodiment, the methods consist essentially of identifying in the amino acid sequence of the polypeptide, or portion thereof, the consensus cyanobacteria DnaE motifs A, B, and C. In another embodiment, the methods consist essentially of identifying in the amino acid sequence of the polypeptide, or portion thereof, the consensus cyanobacteria DnaE motifs A, B, or A and B, and optionally C. The sequence based methods may be combined with other activity assays.

Similarly, the consensus sequences for motifs A, B, and C of the active site of DnaE from gram positive bacteria are, respectively, G-[L/V]-[L/V]-K-X-D-[F/I]-L-G-L-[R/K]-X-L-[T/S], [F/Y/W]-X-X-X-X-[R/K]-F-X-X-Y-[A/G]-F-N-[R/K]-X-H, and P-D-I-D-[L/IN]-D-[F/L/V], wherein X is any amino acid. The arrangement of the motifs, from N-terminus to C-terminus is C-A-B, with a spacing between motifs C-A of about 112-150 amino acids, a spacing between motifs A-B of about 167-190 amino acids, and a consequent spacing between motifs C-B of about 279-340 amino acids. This information provides for a sequence-based method of determining that a polypeptide is a DnaE Pol III α subunit from gram positive bacteria. The method involves determining the amino acid sequence of a candidate polypeptide, or a segment thereof, and identifying therein the amino acid sequence of gram positive DnaE consensus motifs A, B, and C, or C and A, or A and B, or C and B, with the arrangement characteristic of gram positive DnaE. In an alternative embodiment, the methods consist essentially of identifying in the amino acid sequence of the polypeptide, or portion thereof, the consensus gram positive DnaE motifs A, B, and C. In another embodiment, the methods consist essentially of identifying in the amino acid sequence of the polypeptide, or portion thereof, the consensus gram positive DnaE motifs A, B, or A and B, and optionally C. The sequence based methods may be combined with other activity assays.

Similarly, the consensus sequences for motifs A, B, and C of the active site of PolC from gram positive bacteria are, respectively, [L/V]-[L/V]-K-X-D-[A/I]-L-G-H-D-X-P-T, [F/Y]-I-X-S-C-X-[R/K]-I-K-Y-[M/L]-F-P-K-A-H, and P-D-I-D-L-D-F-S, wherein X is any amino acid. The arrangement of the motifs, from N-terminus to C-terminus is C-A-B, with a spacing between motifs C-A of about 124 amino acids, a spacing between motifs A-B of about 173-179 amino acids, and a consequent spacing between motifs C-B of about 297-303 amino acids. This information provides for a sequence-based method of determining that a polypeptide is a PolC from gram positive bacteria. The method involves determining the amino acid sequence of a candidate polypeptide, or a segment thereof, and identifying therein the amino acid sequence of gram positive PolC consensus motifs A, B, and C, or C and A, or A and B, or C and B, with the arrangement characteristic of gram positive PolC. In an alternative embodiment, the methods consist essentially of identifying in the amino acid sequence of the polypeptide, or portion thereof, the consensus PolC motifs A, B, and C. In another embodiment, the methods consist essentially of identifying in the amino acid sequence of the polypeptide, or portion thereof, the consensus PolC motifs A, B, or A and B, and optionally C. The sequence based methods may be combined with other activity assays.

In one aspect, the invention provides compositions and methods for detecting the presence of bacteria in a host. The methods involve analyzing a sample from the host for the presence of a Pol III α subunit. As replicases are critical to the viability of bacteria, Pol III α subunits are extremely useful diagnostic markers that are indicative of the presence of viable bacteria.

In one aspect, the invention provides compositions and methods for screening candidate bioactive agents for the ability to modulate, preferably inhibit, the activity of bacterial DNA Pol III enzymes. In one embodiment, the methods further comprise screening such candidate bioactive agents for the inability to inhibit a human replicase. In one embodiment, the invention provides bioactive agents identified by the screening methods herein. Such bioactive agents obtained by the screening methods described herein find use in the treatment of patients having a bacterial infection.

In addition to identifying and describing the functional motifs of bacterial Pol III α active sites, methods for altering the functionality of bacterial Pol III α subunits, and Pol III replicases comprising the same, through amino acid substitution at a variety of positions within motifs A and B are provided herein. The mutations in motifs A and/or B endow the Pol III α mutants with one or more characteristics distinguishing them from Pol III α subunits not having the one or more mutations. Preferred activity alterations include altered primer discrimination and altered dNTP discrimination.

Accordingly, the invention provides Pol III α mutants having at least one mutation in one or more of motifs A and B, and having functional characteristics different from unmodified Pol III α subunits.

In one aspect, the invention provides Pol III α mutants altered in their ability to discriminate RNA/DNA primers. In one embodiment, Pol III α mutants that preferentially replicate RNA-primed template are provided. Such Pol III α mutants preferably bear one or more mutations in motif B. In another embodiment, Pol III α mutants that preferentially replicate DNA-primed template are provided. Such Pol III α mutants preferably bear one or more mutations in motif B.

In one aspect, the invention provides Pol III α mutants altered in their ability to incorporate labeled nucleotides into primer extension products. In one embodiment, Pol III α mutants having increased ability to incorporate labeled nucleotides into primer extension products are provided. Such Pol III α mutants preferably bear one or more mutations in motif A.

In one aspect, the invention provides Pol III α mutants altered in their ability to incorporate ddNTPs into primer extension products. In a preferred embodiment, Pol III α mutants having increased ability to incorporate ddNTPs into primer extension products are provided. Such Pol III a mutants preferably bear one or more mutations in motif B.

Also provided are Pol III α mutants having more than one activity alteration. In a preferred embodiment, the invention provides Pol III α mutants having an increased ability to incorporate ddNTPs into primer extension products, which also preferentially replicate DNA-primed template.

Also provided herein are methods of producing Pol III α mutants. In a preferred embodiment, the methods involve introducing at least one mutation into one or more of motifs A, B, and C of an unmodified Pol III α. The unmodified Pol III α subunit may be selected from gram negative DnaE, gram positive DnaE, cyanobacteria DnaE, and gram positive PolC. An unmodified Pol III α subunit is preferably characterized as having from N-terminus to C-terminus motifs C, A, B, at spacings characteristic of the particular bacterial type, as disclosed herein.

Additionally provided are modified Pol III replicases comprising Pol III α mutants disclosed herein. The modified Pol III replicases have altered activity relative to unmodified Pol III replicases comprising α subunits not having the one or more mutations. Preferred activity alterations include altered dNTP discrimination, and altered primer discrimination.

Additionally provided are Pol III α subunit isoforms having preferred characteristics. These Pol III α isoforms may be naturally occurring isoforms. Based on the nexus between motif sequences and activities disclosed herein, these isoforms are, for the first time, recognized on the basis of motif sequence as having desirable nucleotide and primer discrimination characteristics, thus making them useful in particular compositions and methods described herein in place of non-naturally occurring Pol III α mutants having the same desirable characteristics, as disclosed herein.

The modified Pol III replicases of the invention may consist of one, two, three, or more components. Included among the modified Pol III replicases of the invention are holoenzyme preparations comprising a Pol III α mutant disclosed herein. Preferred for use in the invention are Pol III α mutants derived from unmodified Pol III α subunits of extremeophiles.

In an especially preferred embodiment, the Pol III α mutant is derived from an unmodified Pol III α subunit of a thermophilic bacterium or thermophilic cyanobacterium. In a preferred embodiment, the thermophilic bacterium is selected from the group consisting of the genera Thermus, Aquifex, Thermotoga, Thermocridis, Hydrogenobacter, Thermosynchecoccus and Thermoanaerobacter. Especially preferred are Aquifex aeolicus, Aquifex pyogenes, Thermus thermophilus, Thermus aquaticus, Thermotoga neapolitana and Thermotoga maritima.

In one aspect, the invention directed to the use of modified Pol III replicases in compositions and methods for nucleic acid replication, including methods of DNA amplification, such as PCR, and DNA sequencing.

Accordingly, in one aspect, the invention provides a method for replicating a nucleic acid molecule, which method comprises subjecting the nucleic acid molecule to a replication reaction in a replication reaction mixture comprising a modified Pol III replicase disclosed herein. In one embodiment, the modified Pol III replicase is a single component Pol III replicase. In another embodiment, the modified Pol III replicase is a two component Pol III replicase. In another embodiment, the modified Pol III replicase comprises three or more components. In one embodiment, a combination of modified Pol III replicases is used in the replication reaction mixture. In one embodiment, a single component or two component Pol III replicase is used in combination with one or more modified Pol III replicases. In one embodiment, a type I single subunit repair DNA polymerase is used in combination with one or more modified Pol III replicases.

In a preferred embodiment, the nucleic acid molecule replicated is a DNA molecule. In a further preferred embodiment, the DNA molecule is double stranded. In a further preferred embodiment, the double stranded DNA molecule is a linear DNA molecule. In other embodiments, the DNA molecule is non-linear, for example circular or supercoiled DNA.

In a preferred embodiment, the method for replicating a nucleic acid molecule is a sequencing method useful for sequencing a nucleic acid molecule, preferably DNA. In a preferred embodiment, the method involves subjecting the nucleic acid molecule to a sequencing reaction in a sequencing reaction mixture. The sequencing reaction mixture comprises a modified Pol III replicase, preferably a single component modified Pol III replicase disclosed herein. The modified Pol III replicase used comprises a mutant Pol III disclosed herein and has an increased ability to incorporate ddNTPs into primer extension products. Preferably the modified Pol III replicase lacks 3′-5′ exonuclease activity capable of removing 3′ terminal ddNTPS in the sequencing reaction mixture. In a preferred embodiment, the modified Pol III replicase comprises a Pol III c mutant derived from an unmodified dnaE α subunit, preferably of the genus Thermus or Aquifex, preferably of the species Thermus thermophilus, Thermus aquaticus, or Aquifex aeolicus.

In another preferred embodiment, the method for replicating a nucleic acid molecule is an amplification method useful for amplifying a nucleic acid molecule, preferably DNA. In a preferred embodiment, the method involves subjecting the nucleic acid molecule to an amplification reaction in an amplification reaction mixture. The amplification reaction mixture comprises a modified Pol III replicase disclosed herein. The modified Pol III replicase used comprises a mutant Pol III α disclosed herein and has an increased ability to incorporate labeled dNTPs into primer extension products. The modified Pol III replicase preferably possesses 3′-5′ exonuclease activity in the amplification reaction mixture.

In a preferred embodiment, the amplification method is a thermocycling amplification method useful for amplifying a nucleic acid molecule, preferably DNA, which is preferably double stranded, by a temperature-cycled mode. In a preferred embodiment, the method involves subjecting the nucleic acid molecule to a thermocycling amplification reaction in an thermocycling amplification reaction mixture. The thermocycling amplification reaction mixture comprises a thermostable modified Pol III replicase. In a preferred embodiment, the thermostable modified Pol III replicase possesses 3′-5′ exonuclease activity in the thermocycling amplification reaction mixture. In a preferred embodiment, the thermostable modified Pol III replicase comprises a Pol III α mutant derived from an unmodified dnaE α subunit, preferably of the genus Thermus or Aquifex, preferably of the species Thermus thermophilus, Thermus aquaticus, or Aquifex aeolicus. In a preferred embodiment, the thermocycling amplification reaction mixture further comprises thermostabilizers, as disclosed herein.

In a preferred embodiment, the thermocycling amplification method is a PCR method, useful for amplifying a nucleic acid molecule, preferably DNA, which is preferably double stranded, by PCR.

In a preferred embodiment, the method involves subjecting the nucleic acid molecule to PCR in a PCR reaction mixture. The PCR reaction mixture comprises a thermostable modified Pol III replicase.

In a preferred embodiment, the invention provides methods for fast PCR. In a preferred embodiment, the method involves subjecting the nucleic acid molecule to fast PCR in a fast PCR reaction mixture. The fast PCR reaction mixture comprises a thermostable modified Pol III replicase.

In a preferred embodiment, the invention provides methods for long range PCR. In a preferred embodiment, the method involves subjecting the nucleic acid molecule to long range PCR in a long range PCR reaction mixture. The long range PCR reaction mixture comprises a thermostable modified Pol III replicase.

In one aspect, the invention provides methods for simultaneous sequencing and amplification of DNA molecules in one homogenous reaction mixture, comprising subjecting the DNA molecules to a sequencing/amplification reaction in a sequencing/amplification reaction mixture comprising a modified Pol III replicase and a thermostable type I single subunit repair DNA polymerase.

In a preferred embodiment the sequencing/amplification reaction mixture used for a simultaneous sequencing/amplification reaction involving one or more high temperature denaturation steps comprises two RNA primers (forward and reverse) to drive the sequencing template amplification by the modified Pol III replicase, and a single DNA primer to drive the sequencing reaction by the repair type DNA polymerase. The repair type DNA polymerase preferably carries a mutated motif B sequence in which the conserved phenylalanine residue is replaced by a tyrosine residue. The modified Pol III replicase has an increased preference for RNA-primed template and preferably comprises one or more mutations in motif B. In one embodiment, the mixture further comprises stabilizers that contribute to the thermostability of the modified Pol III replicase.

In an alternative embodiment, a second modified Pol III replicase having increased ability to incorporate ddNTPs into primer extension products is used in place of the repair type DNA polymerase in a simultaneous sequencing/amplification reaction. The second modified Pol III replicase preferably comprises one or more mutations in motif B. In a preferred embodiment, the modified Pol III replicase additionally has increased preference for DNA-primed template.

In an alternative embodiment, the amplification and sequencing reactions are not simultaneous. In this embodiment, RNA primers and DNA primers, and/or modified Pol III replicase and repair type DNA polymerase (or second modified Pol III replicase) are added sequentially to the same reaction mixture.

In one aspect, the invention provides replication reaction mixtures for nucleic acid replication, which mixtures comprise modified Pol III replicases disclosed herein. In a preferred embodiment, a replication reaction mixture is useful for DNA replication. In one embodiment, the modified Pol III replicase is a single component modified Pol III replicase. In another embodiment, the modified Pol III replicase is a two component modified Pol III replicase. In another embodiment, the modified Pol III replicase comprises three or more components. In another embodiment, a combination of modified Pol III replicases are used in a replication reaction mixture.

In a preferred embodiment, the replication reaction mixture is a sequencing reaction mixture useful for nucleic acid sequencing, preferably DNA sequencing. The sequencing reaction mixture comprises a modified Pol III replicase, preferably a single component modified Pol III replicase disclosed herein. The modified Pol III replicase used comprises a mutant Pol III α disclosed herein and has an increased ability to incorporate ddNTPs into primer extension products. Preferably the modified Pol III replicase lacks 3′-5′ exonuclease activity capable of removing 3′ terminal ddNTPs in the sequencing reaction mixture. In a preferred embodiment, the modified Pol III replicase comprises a Pol III α mutant derived from an unmodified dnaE α subunit, preferably of the genus Thermus or Aquifex, preferably of the species Thermus thermophilus, Thermus aquaticus, or Aquifex aeolicus.

In another preferred embodiment, the replication reaction mixture is an amplification reaction mixture useful for nucleic acid amplification, preferably DNA amplification. The amplification reaction mixture comprises a modified Pol III replicase disclosed herein. The modified Pol III replicase used comprises a mutant Pol III α disclosed herein and has an increased ability to incorporate labeled dNTPs into primer extension products. The modified Pol III replicase preferably possesses 3′-5′ exonuclease activity in the amplification reaction mixture.

In a preferred embodiment, the amplification reaction mixture is a thermocycling amplification reaction mixture useful for amplifying nucleic acid, preferably DNA, which is preferably double stranded, by a temperature-cycled mode. Preferably, the thermocycling amplification reaction mixture comprises a thermostable modified Pol III replicase. In a preferred embodiment, the thermostable modified Pol III replicase possesses 3′-5′ exonuclease activity in the thermocycling amplification reaction mixture. In a preferred embodiment, the thermostable modified Pol III replicase comprises a Pol III α mutant derived from an unmodified dnaE α subunit, preferably of the genus Thermus or Aquifex, preferably of the species Thermus thermophilus, Thermus aquaticus, or Aquifex aeolicus. In a preferred embodiment, the thermocycling amplification reaction mixture further comprises thermostabilizers, as disclosed herein.

In a preferred embodiment, the thermocycling amplification reaction mixture is a polymerase chain reaction mixture (“PCR mixture”) useful for amplifying nucleic acids, preferably DNA, which is preferably double stranded, by PCR. Preferably, the PCR mixture comprises a thermostable modified Pol III replicase.

In a preferred embodiment, the invention provides PCR mixtures that are fast PCR mixtures useful in fast PCR methods. Preferably, a fast PCR mixture comprises a thermostable modified Pol III replicase.

In a preferred embodiment, the invention provides PCR mixtures that are long range PCR mixtures useful in long range PCR methods. Preferably, a long range PCR mixture comprises a thermostable modified Pol III replicase.

In one aspect, the invention provides nucleic acid replication reaction tubes, which comprise nucleic acid replication reaction mixtures disclosed herein. Tubes comprising a replication reaction mixture are tubes that contain a reaction mixture.

In a preferred embodiment, the nucleic acid replication reaction tubes are sequencing reaction tubes, which comprise a sequencing reaction mixture disclosed herein.

In another preferred embodiment, the nucleic acid replication reaction tubes are amplification reaction tubes, which comprise an amplification reaction mixture disclosed herein.

In a preferred embodiment, the amplification reaction tubes are thermocycling amplification reaction tubes, which comprise a thermocycling amplification reaction mixture disclosed herein.

In a preferred embodiment, the thermocycling amplification reaction tubes are PCR tubes, which comprise a PCR reaction mixture disclosed herein.

In a preferred embodiment, the invention provides PCR tubes that are fast PCR tubes, which comprise a fast PCR reaction mixture disclosed herein.

In a preferred embodiment, the invention provides PCR tubes that are long range PCR tubes, which comprise a long range PCR reaction mixture disclosed herein.

In one aspect, the invention provides nucleic acid replication kits useful for nucleic acid replication, which kits comprise modified Pol III replicases disclosed herein. In a preferred embodiment, a replication kit comprises a replication reaction mixture disclosed herein. The replication reaction mixture of the kit may be free of modified Pol III replicase, and may require addition of modified Pol III replicase prior to use.

In a preferred embodiment, the nucleic acid replication kit is a sequencing kit useful for nucleic acid sequencing, preferably DNA sequencing.

In another preferred embodiment, the nucleic acid replication kit is an amplification kit useful for nucleic acid amplification, preferably DNA amplification.

In a preferred embodiment, the amplification kit is a thermocycling amplification kit useful for amplifying nucleic acids, preferably DNA, which is preferably double stranded, by a temperature-cycled mode.

In a preferred embodiment, the thermocycling amplification kit is a PCR kit for amplifying nucleic acids, preferably DNA, which is preferably double stranded, by PCR.

In a preferred embodiment, the invention provides PCR kits that are fast PCR kits.

In a preferred embodiment, the invention provides PCR kits that are long range PCR kits.

### BRIEF DESCRIPTION OF THE DRAWINGS

FIG. 1 schematically compares the arrangement and spacing of motifs A, B and C in a variety of gram negative and gram positive DNA polymerase subunits, as well as in human, archaebacterial, and bacteriophage DNA polymerase subunits.

FIG. 2 provides preferred and secondary substitutions within motif A to provide different functional characteristics in gram negative DNAe pol III alpha.

FIG. 3 provides preferred and secondary substitutions within motif B to provide different functional characteristics in gram negative DNAe pol III alpha.

FIG. 4 provides preferred and secondary substitutions within motif A To provide different functional characteristics in gram positive DNAe pol III alpha.

FIG. 5 provides preferred and secondary substitutions within motif B to provide different functional characteristics in gram positive DNAe pol III alpha.

FIG. 6 provides preferred and secondary substitutions within motif A provide different functional characteristics in gram positive PolC pol III alpha.

FIG. 7 provides preferred and secondary substitutions within motif B to provide different functional characteristics in gram positive PolC pol III alpha.

FIG. 8 provides preferred and secondary substitutions within motif A to provide different functional characteristics in cyanobacteria DNAe pol III alpha.

FIG. 9 provides preferred and secondary substitutions within motif B to provide different functional characteristics in cyanobacteria DNAe pol III alpha.

FIG. 10 shows the results of a time course primer extension assay using Thermus thermophilus (T.th) alpha subunit.

FIG. 11 shows the results of a time course primer extension assay using Thermotoga maritima alpha subunit.

FIG. 12 provides a proposed active site model for DnaE-type alpha subunits of DNA pol III based on the T.th DnaE sequence.

FIG. 13 provides a proposed active site model for PolC-type alpha subunits of DNA pol III based on the T. maritima PolC sequence.

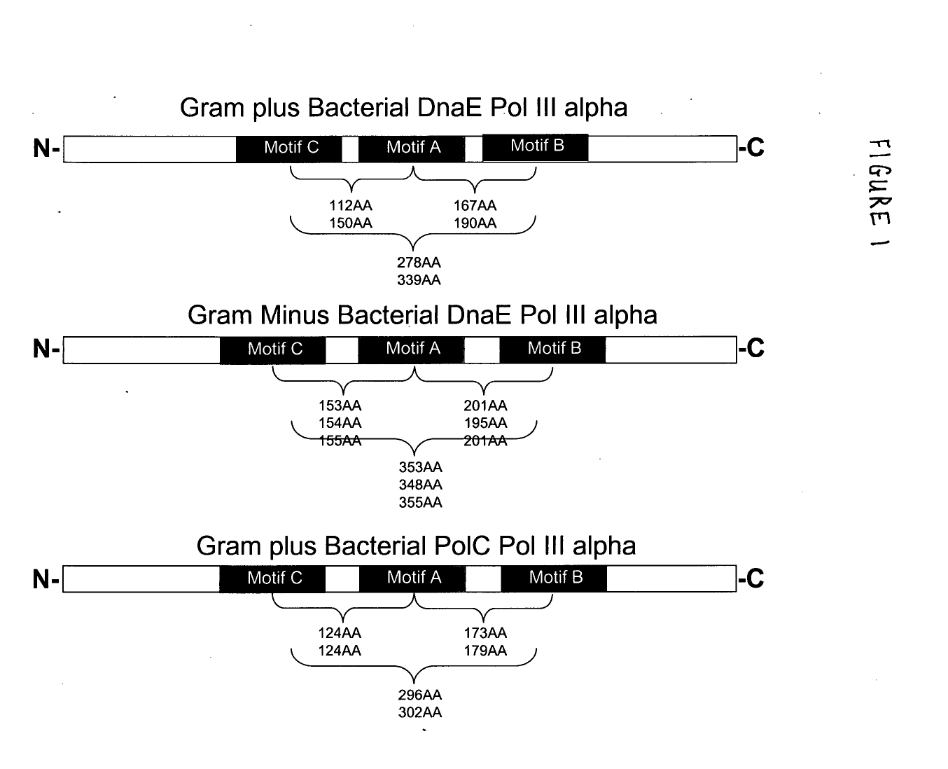

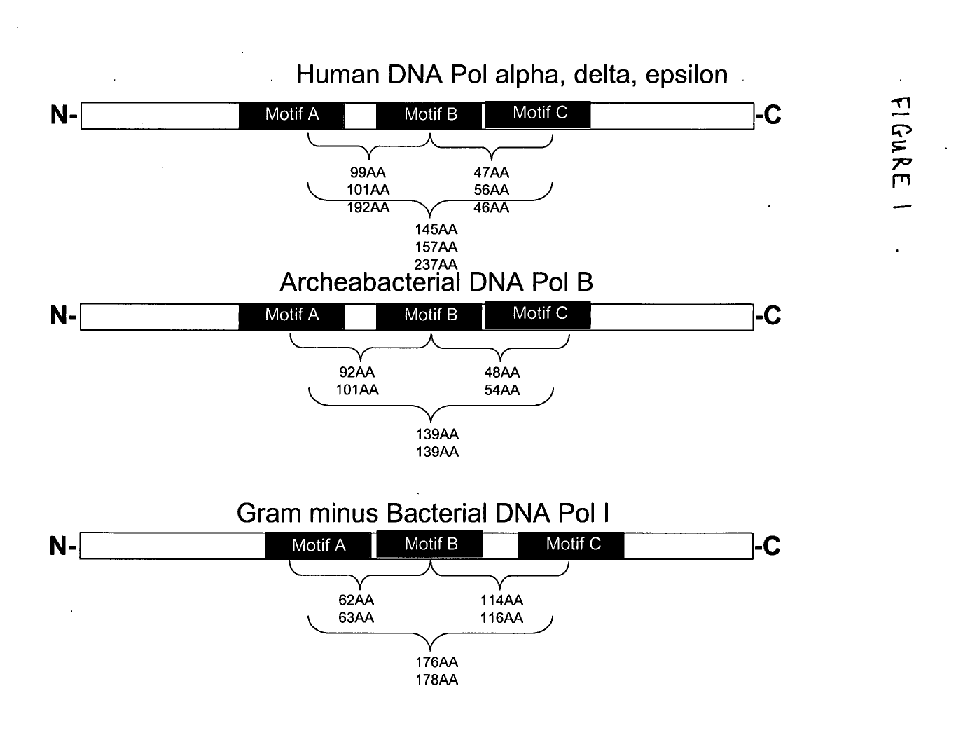

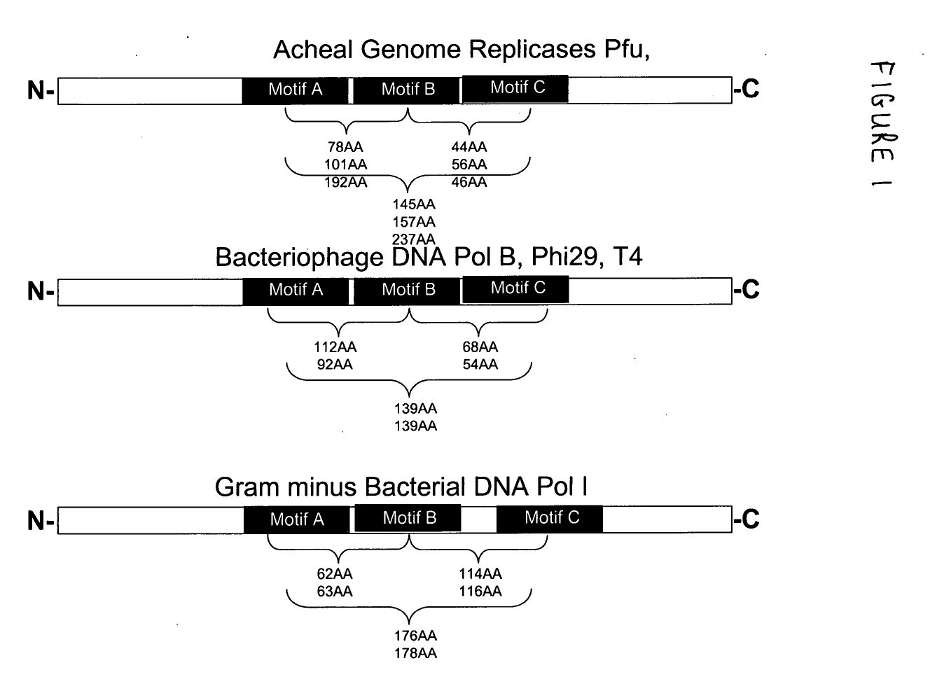

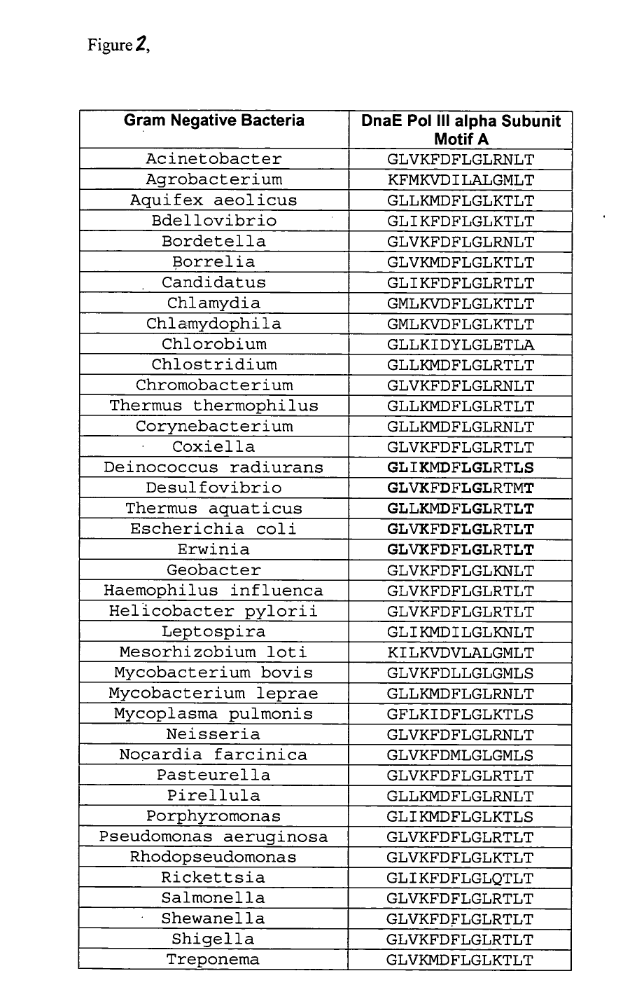

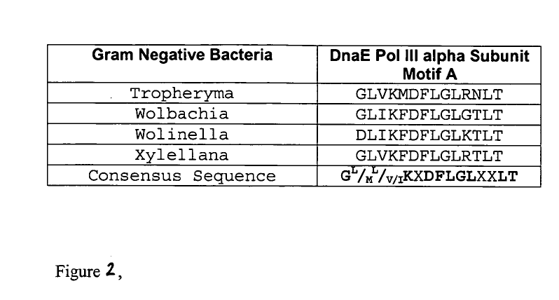

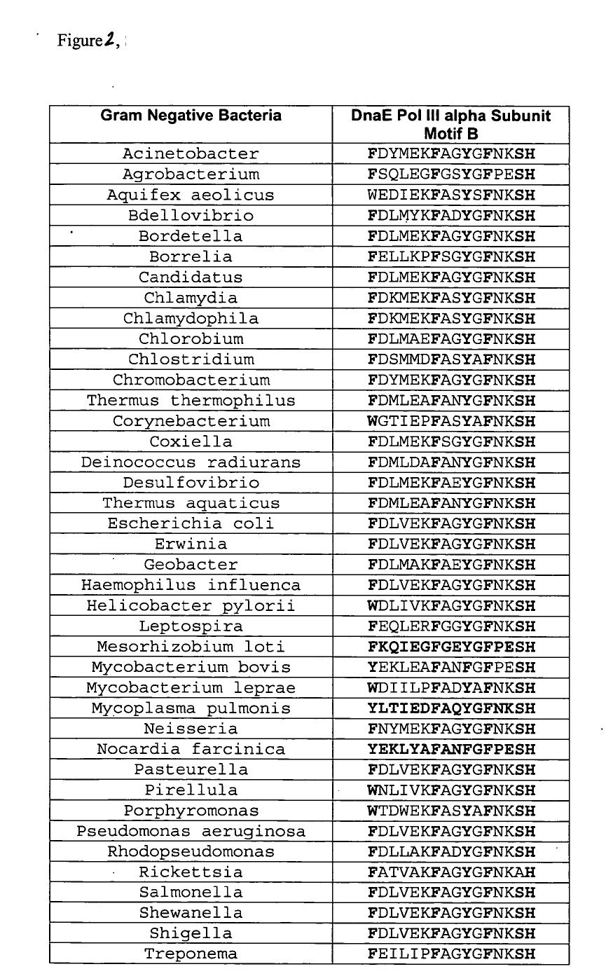

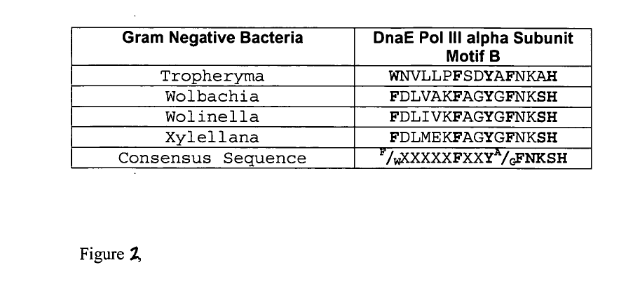

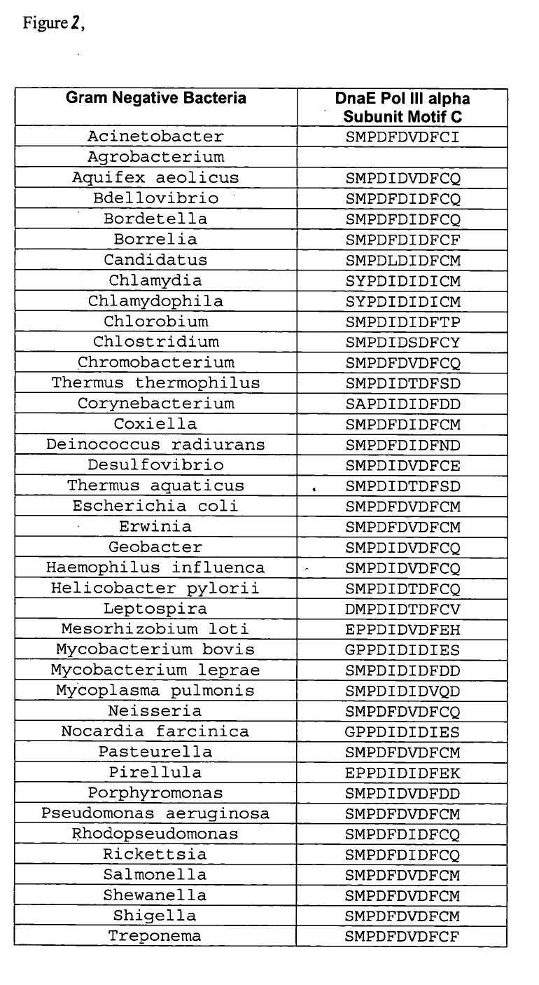

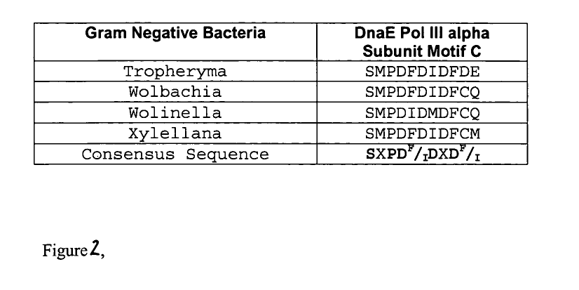

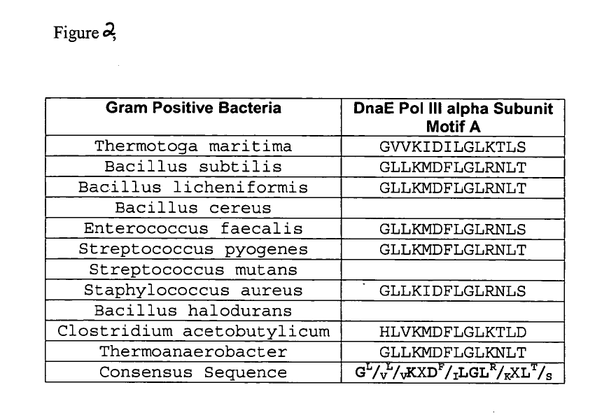

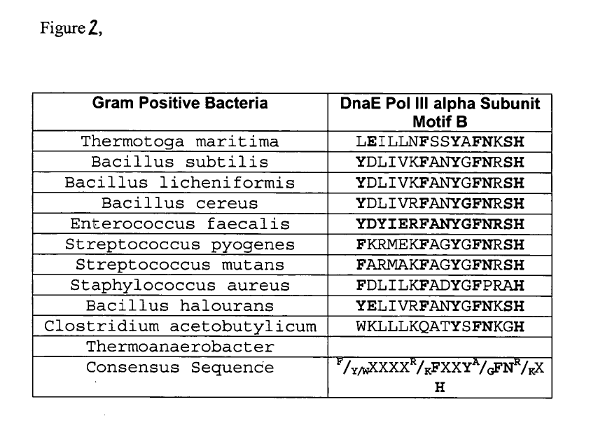

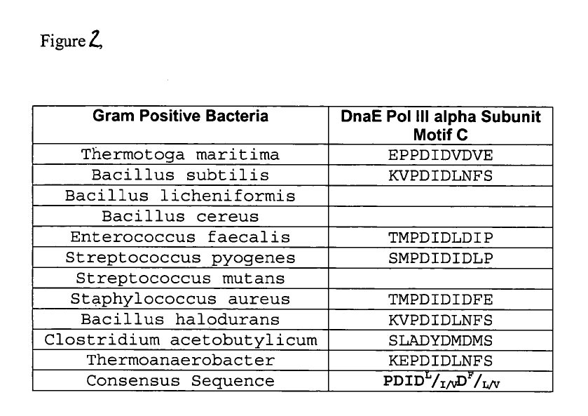

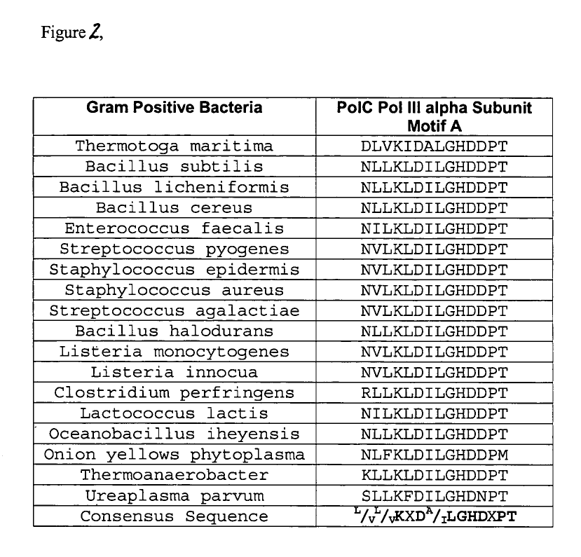

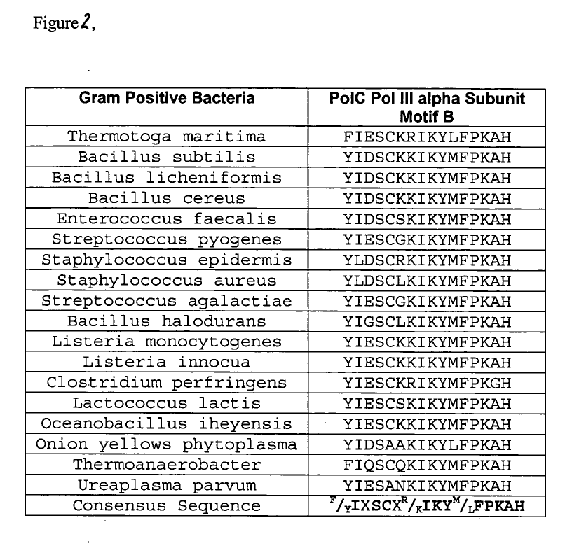

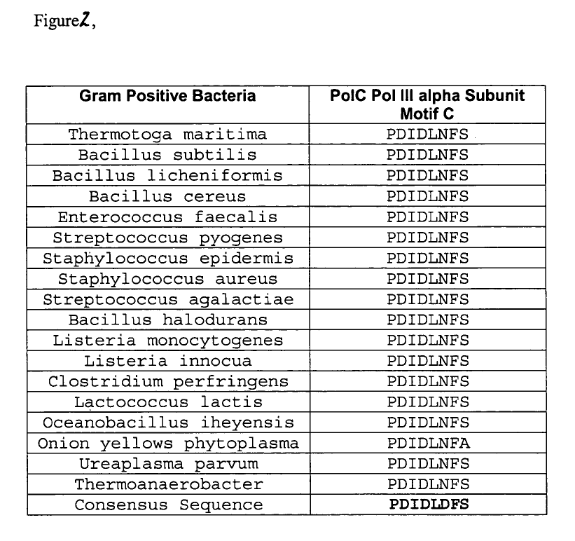

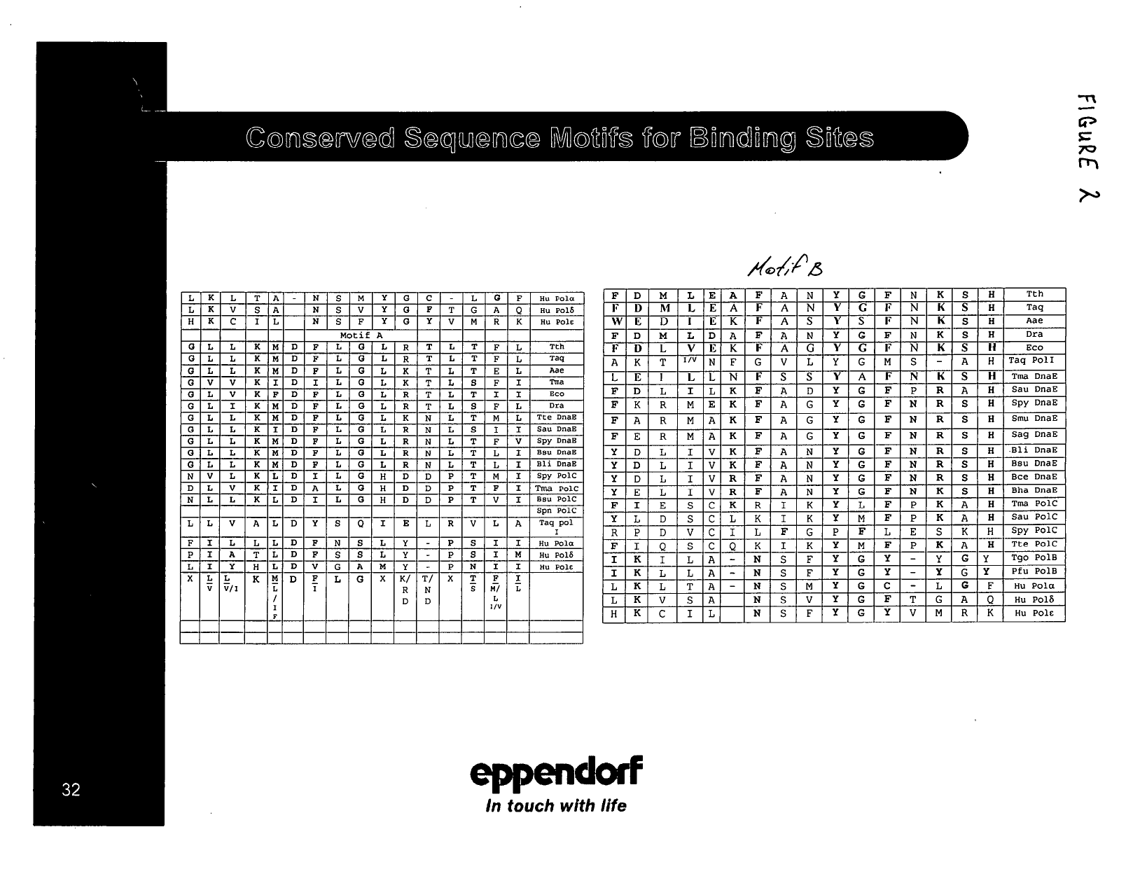

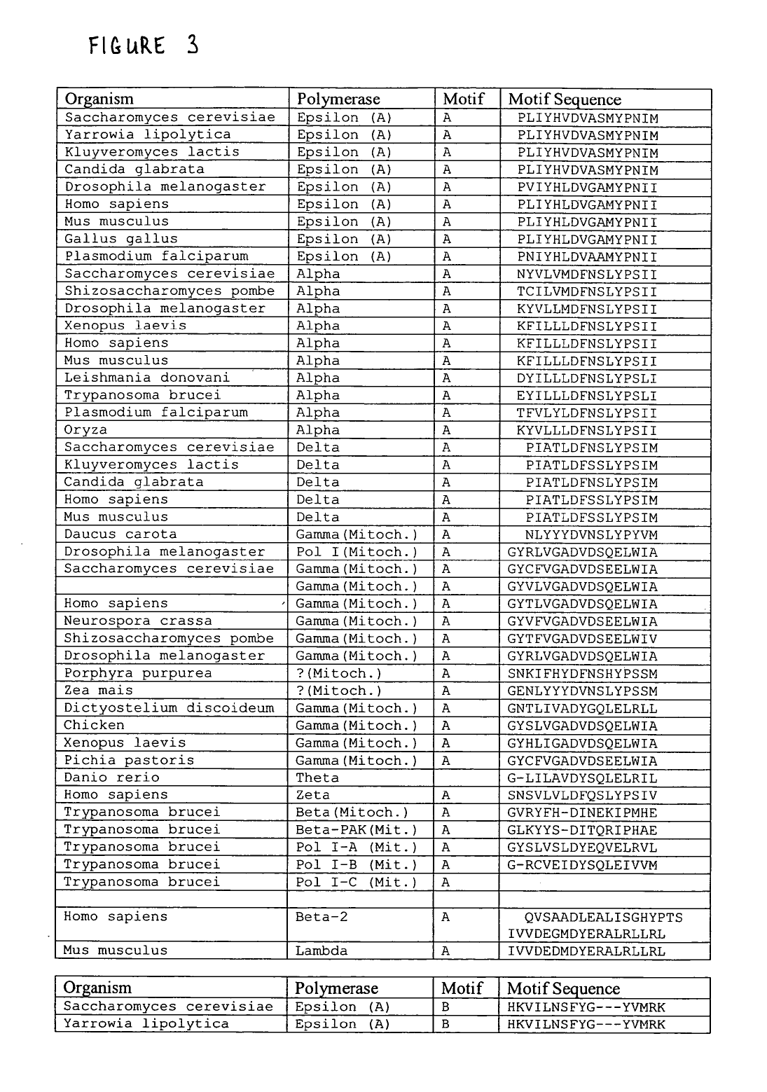

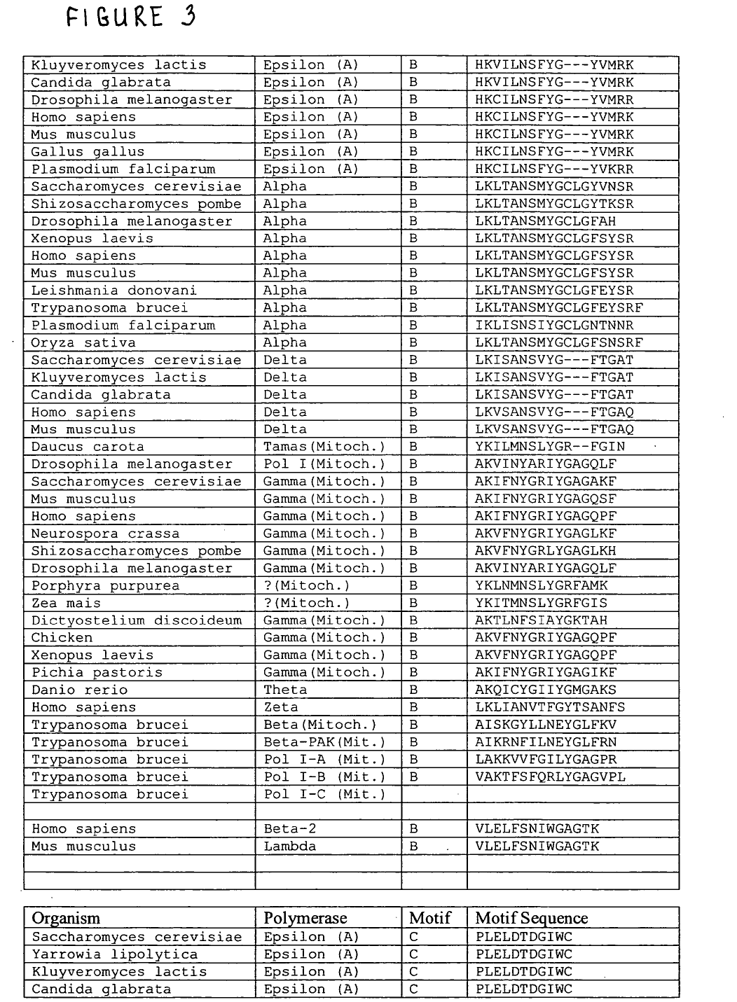

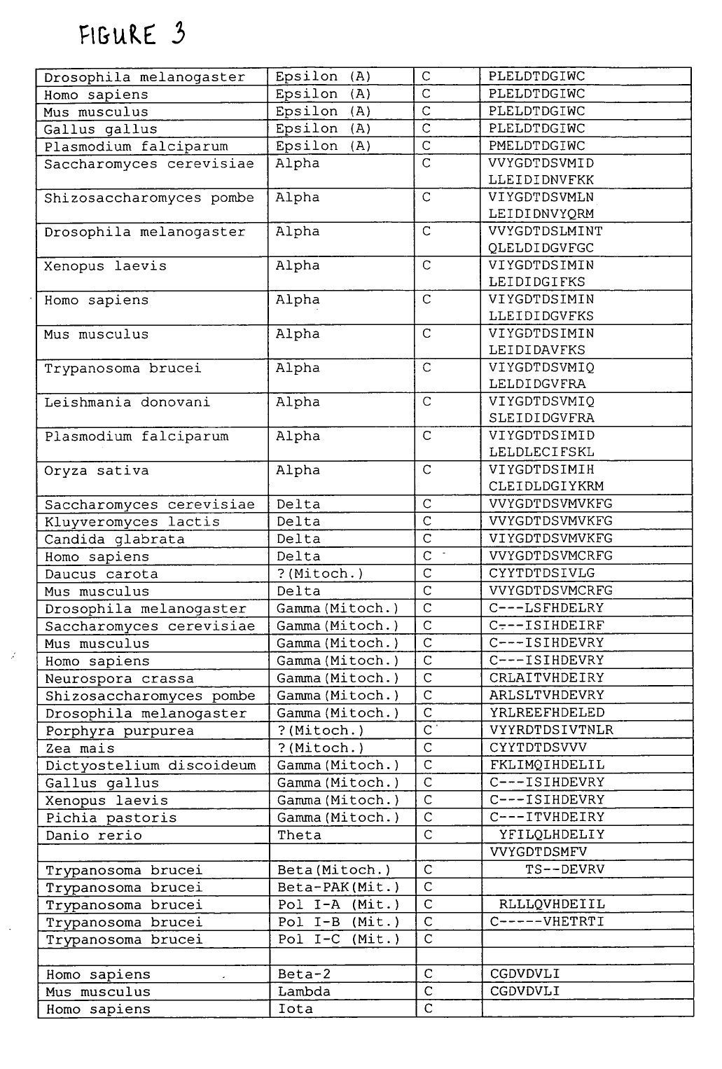

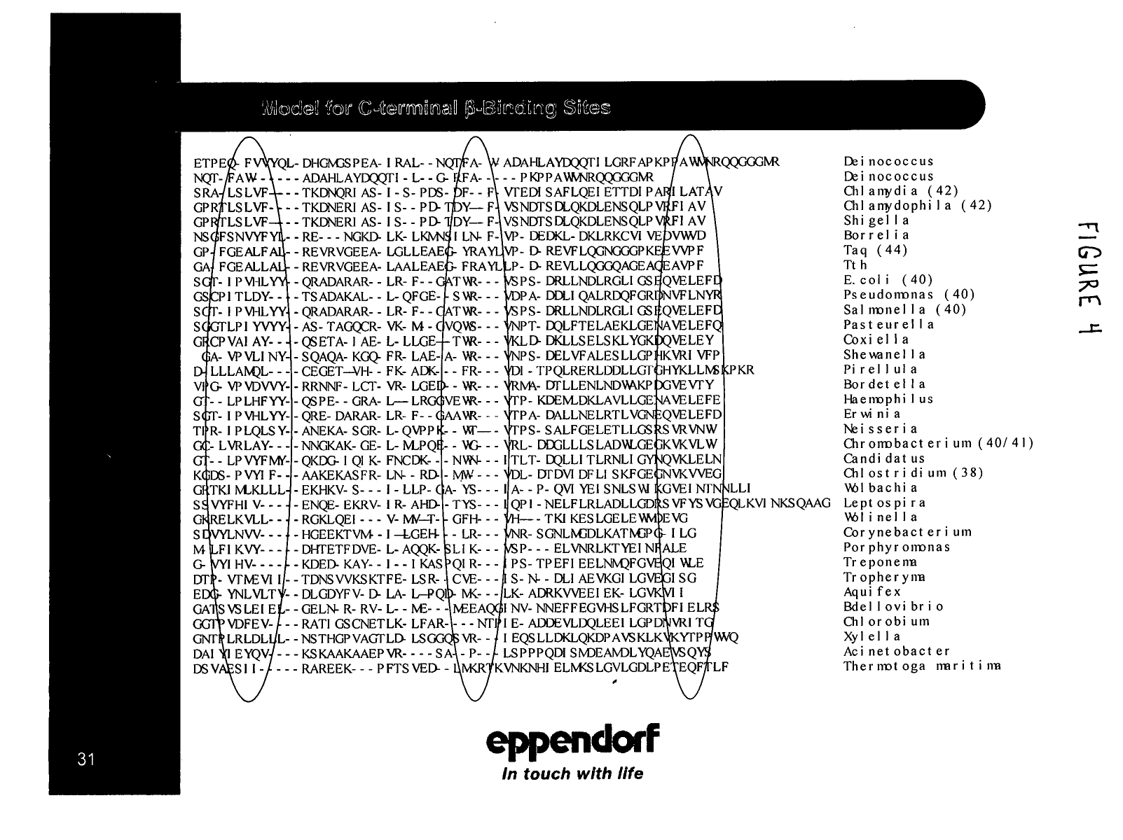

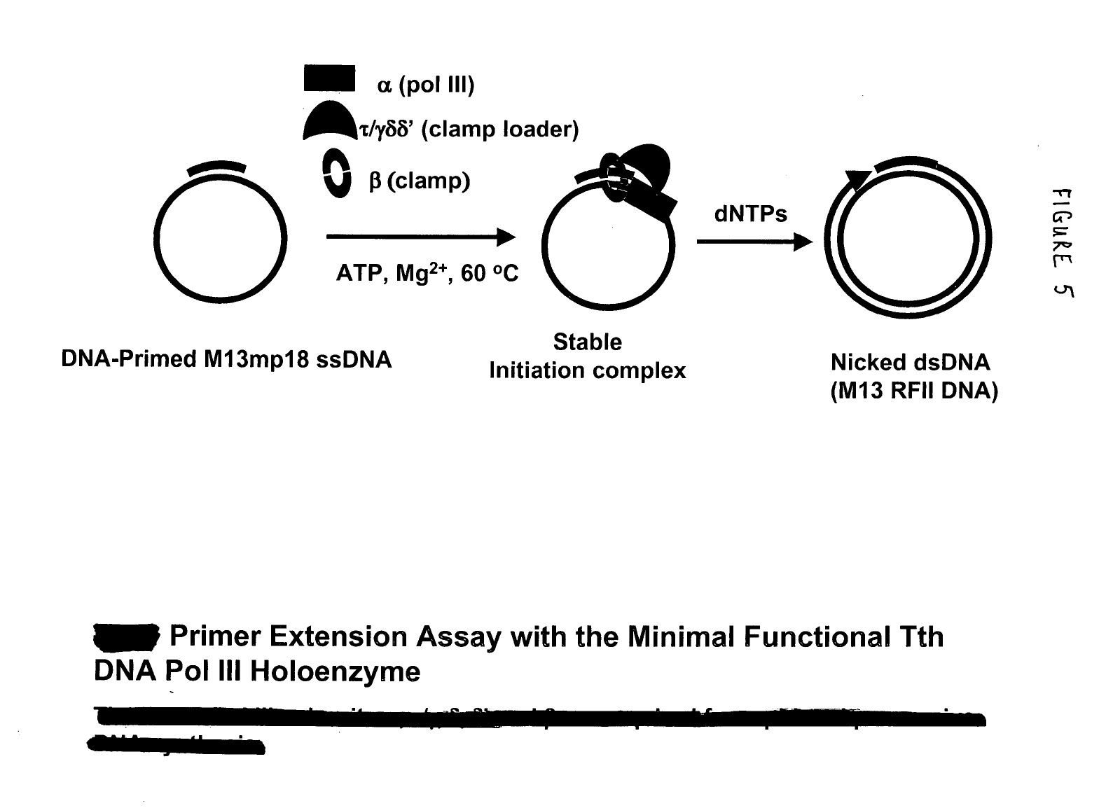

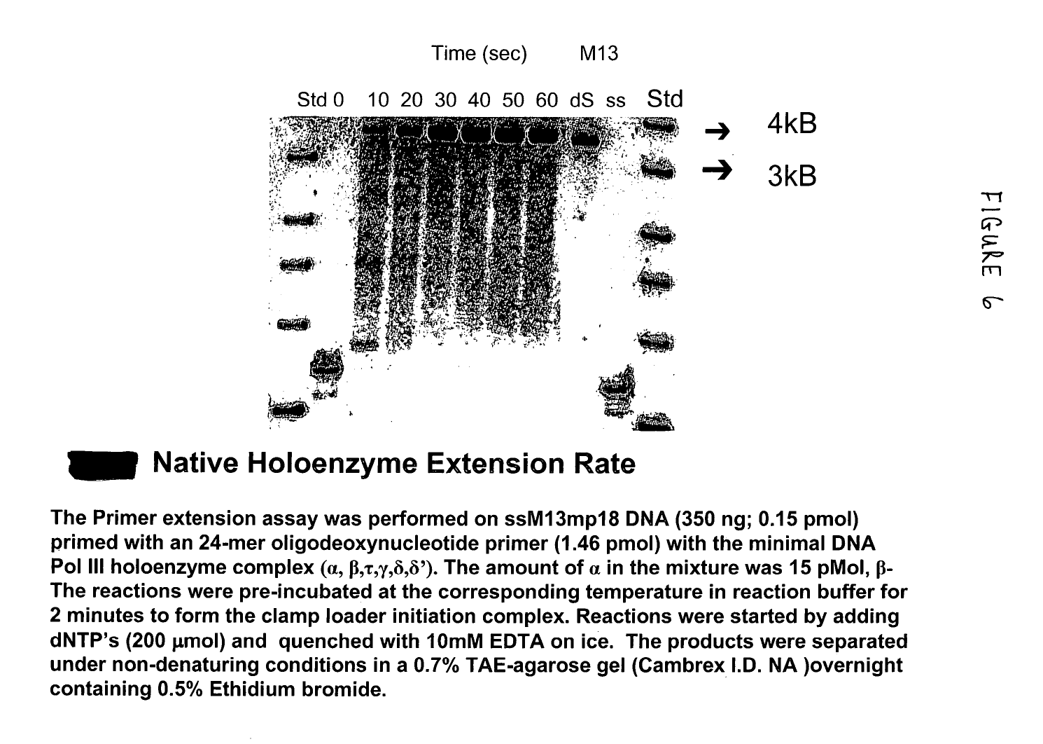

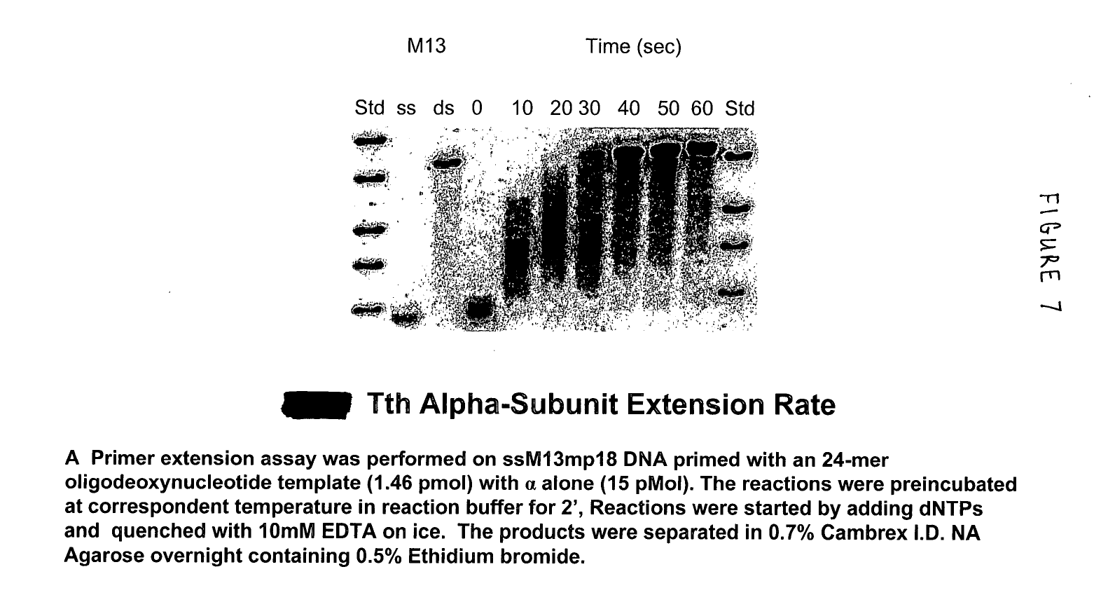

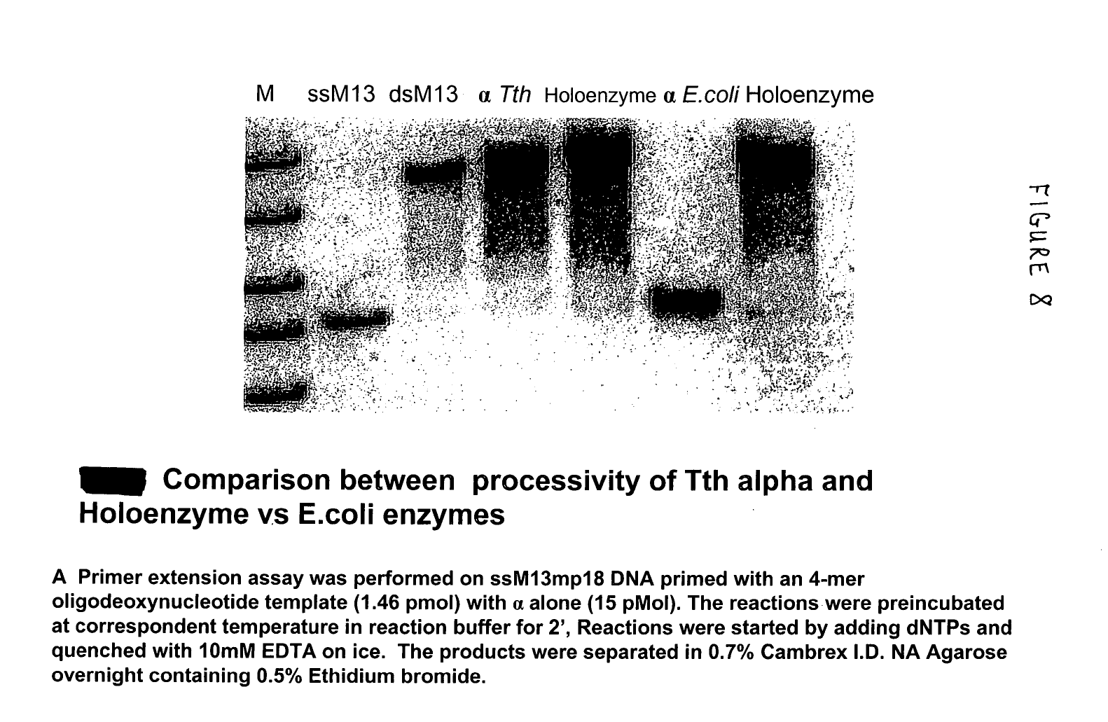

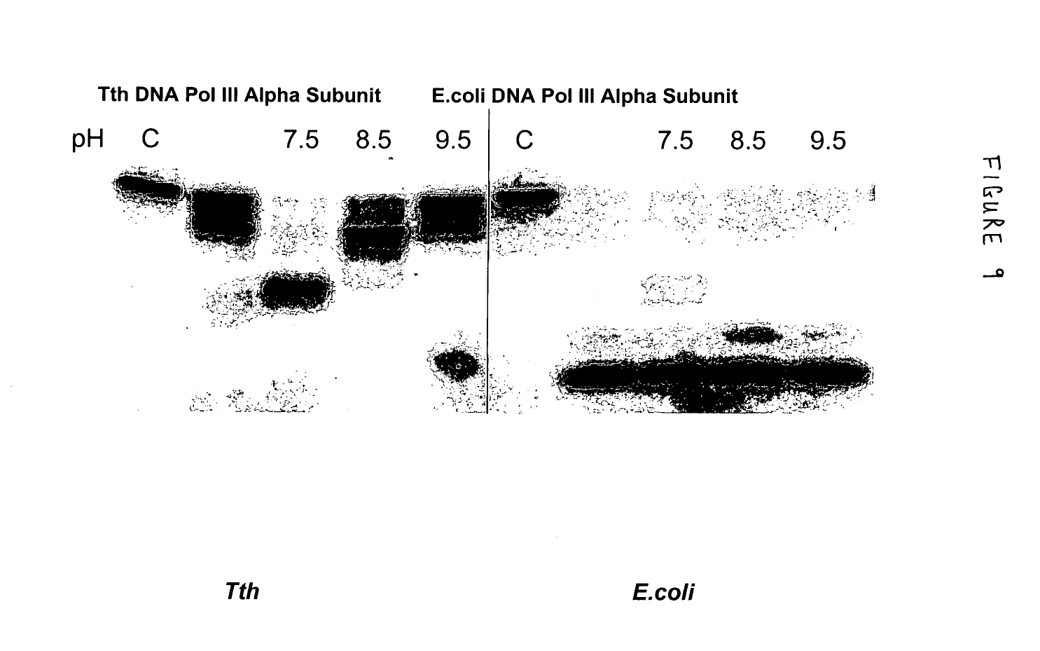

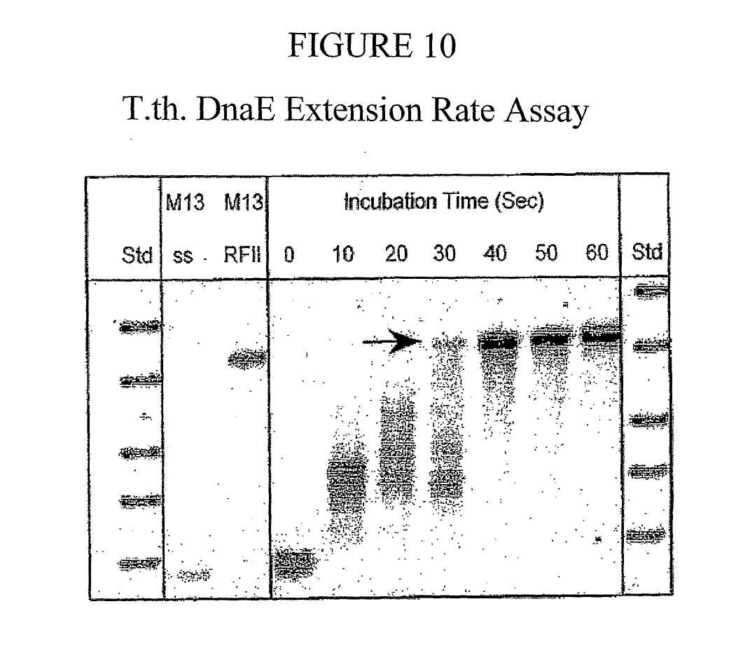

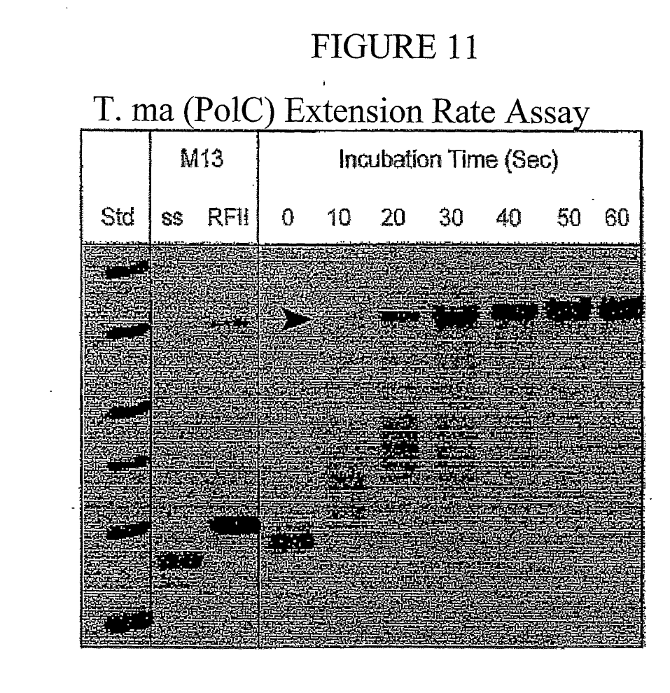

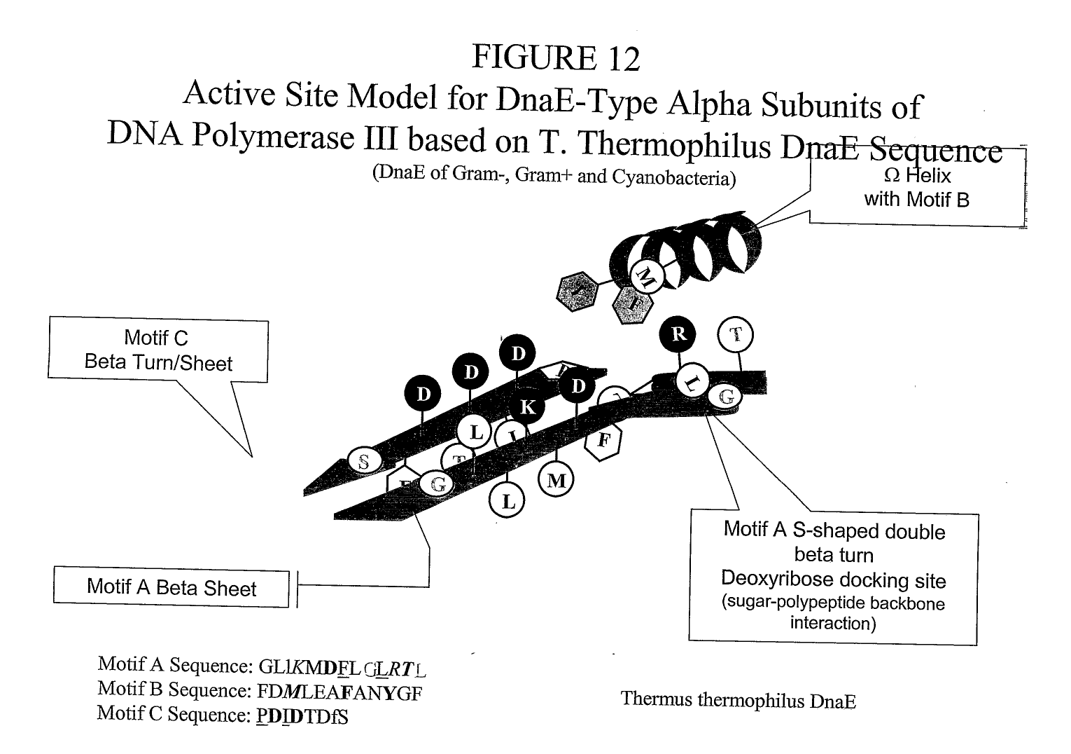

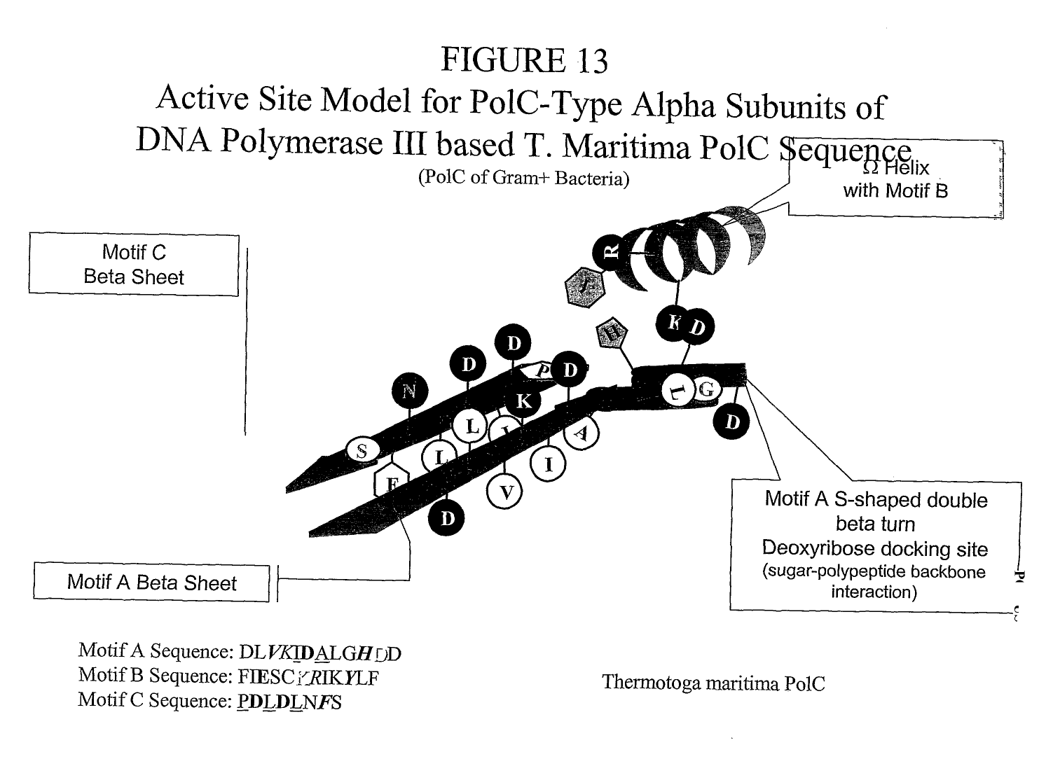

### DETAILED DESCRIPTION OF THE INVENTION

“Labeled nucleotides” as used herein refers to nucleotides having a label attached thereto. Examples of labels and labeled nucleotides are well known in the art. The label is typically a hydrophobic molecule, which is frequently attached to the base moiety of the nucleotide. The label typically provides for detection of the nucleotide, or alters the characteristics thereof.

As used herein “thermostable” refers to a DNA polymerase which is resistant to inactivation by heat. DNA polymerases, including the modified Pol III replicases disclosed herein, synthesize the formation of a DNA molecule complementary to a single-stranded DNA template by extending a primer in the 5′ to 3′ direction. As used herein, a thermostable DNA polymerase is more resistant to heat inactivation than a thermolabile DNA polymerase. However, a thermostable DNA polymerase is not necessarily totally resistant to heat inactivation, and, thus, heat treatment may reduce the DNA polymerase activity to some extent. A thermostable DNA polymerase typically will also have a higher optimum temperature for synthetic function than thermolabile DNA polymerases. Thermostable DNA polymerases are typically isolated from thermophilic organisms, for example, thermophilic bacteria.

As used herein “thermolabile” refers to a DNA polymerase which is not resistant to inactivation by heat. For example, T5 DNA polymerase, the activity of which is totally inactivated by exposing the enzyme to a temperature of 90° C. for 30 seconds, is considered to be a thermolabile DNA polymerase. As used herein, a thermolabile DNA polymerase is less resistant to heat inactivation than is a thermostable DNA polymerase. A thermolabile DNA polymerase typically is also likely to have a lower optimum temperature than a thermostable DNA polymerase. Thermolabile DNA polymerases are typically isolated from mesophilic organisms, for example, mesophilic bacteria or eukaryotes, including certain animals.

### Classification Methods and Activity Determinations

In one aspect, the invention provides protein classification and activity determination methods. These methods are based on the discovery of previously unrecognized protein motifs A, B, and C, and unusual arrangements thereof, that are conserved in bacterial DNA Pol III α subunits. These signature amino acid sequence motifs, and the arrangement thereof, including their spacing, are critical to the function of DNA Pol III, and may be used determinatively.

The amino acid sequences of motifs A, B, and C of DNA Pol III α subunits vary somewhat between gram negative bacteria, gram positive bacteria, and cyanobacteria, and between bacterial Pol III enzyme types (DnaE and PolC), thus allowing differentiation based on sequence determination. Further, the spacings between motifs A, B, and C of DNA Pol III α subunits vary between gram negative bacteria, gram positive bacteria, and cyanobacteria, and between Pol III enzyme types, thus allowing differentiation based on motif arrangement in sequence. As disclosed herein, functional motifs A, B, and C, analogous to those previously identified in non-Pol III DNA polymerases, are present in DNA Pol III α subunits of gram negative bacteria, gram positive bacteria, and cyanobacteria. Notably, the order of these motifs in the Pol III α subunits differs from the order of the motifs in non-Pol III polymerases. In bacterial Pol III α subunits, the motifs are arranged, from N- to C-terminus, in the order C-A-B.

In a preferred embodiment, the invention provides methods for classifying a polypeptide as a DNA polymerase, comprising comparing the amino acid sequence of the polypeptide, or a portion thereof, to the consensus amino acid sequences of bacterial DNA Pol III motifs A, B, and C. In a preferred embodiment, the methods involve identifying all three motifs, namely A, B, and C, in the polypeptide. The methods further involve determining the arrangement of the three motifs in the polypeptide. The methods further comprise determining the amino acid spacing between the three motifs. If all three motifs are identified in a polypeptide, and the motifs are arranged in the order, from amino terminus to carboxyl terminus, C-A-B, and the three motifs are spaced from each other by distances within the characteristic spacing range of the consensus motifs in the bacterial DNA Pol 111, then it is determined that the polypeptide is a DNA polymerase.

In an alternative embodiment, the methods involve determining the amino acid sequence of a candidate polypeptide, or a segment thereof, and identifying therein the amino acid sequence of bacterial Pol III consensus motifs C and A, or A and B, or C and B, with the arrangement characteristic of bacterial Pol III.

In an alternative embodiment, the methods consist essentially of identifying in the amino acid sequence of the polypeptide, or portion thereof, the consensus bacterial DNA Pol III motifs A, B, and C. In another embodiment, the methods consist essentially of identifying in the amino acid sequence of the polypeptide, or portion thereof, the consensus bacterial DNA Pol III motifs A, B, or A and B, and optionally C. Additional assays may be combined with such sequence-based methods.

As an alternative to sequence determination, high stringency hybridization to a probe complementary to consensus bacterial DNA motifs A, B, or C may be used to identify the presence of consensus sequences.

In one embodiment, the consensus sequences of bacterial Pol III motifs A, B, and C are, respectively, [L/V/M]-[L/V/I]-K-X-D-[F/A/I]-L-G-[L/H]-X-X-[L/P]-[T/S], [F/Y/W]-X-X-X-X-X-[F/R/K/]-X-X-Y-[A/G/M/L]-F-[N/P]-[R/K]-X-H, and P-D-[F/I]-D-X-D-[F/I/L/V], wherein X is any amino acid. The motifs are arranged, from amino terminus to carboxyl terminus, in the order C-A-B. The spacing between motif C and A ranges from about 112 to about 155 amino acids. The spacing between motif A and motif B ranges from about 167 to about 201 amino acids. The spacing from motif C to motif B ranges from about 270 to about 356 amino acids.

In a preferred embodiment, the methods comprise comparing the sequence of a polypeptide to one, two, three, or four sets of consensus sequences of motifs A, B, and C, wherein the set(s) of consensus sequences is selected from the set of motif consensus sequences for gram negative bacteria dnaE gene products, the set of motif consensus sequences for gram positive bacteria dnaE gene products, the set of consensus sequences for gram positive bacteria polC gene products, and the set of consensus sequences for cyanobacteria dnaE gene products. In a preferred embodiment, the methods involve identifying all three motifs, namely A, B, and C of a particular set of consensus motifs, in a polypeptide. The methods preferably further involve determining the arrangement of the three motifs in the polypeptide. The methods preferably further comprise determining the amino acid spacing between the three motifs. If all three motifs of a particular set of consensus motifs are identified in a polypeptide, and the motifs are arranged in the order, from amino terminus to carboxyl terminus, C-A-B, and the three motifs are spaced from each other by distances within the range characteristic of the particular set of consensus motifs, then it is determined that the polypeptide is a DNA polymerase of the corresponding type. Accordingly, the polypeptide may be used as a Pol III α subunit in compositions and methods herein. Additionally, the polypeptide may be used as the parent molecule for the derivation of a Pol III α mutant having preferred characteristics.

In an alternative embodiment, the methods involve determining the amino acid sequence of a candidate polypeptide, or a segment thereof, and identifying therein the amino acid sequence of bacterial Pol III consensus motifs C and A, or A and B, or C and B, from a particular set of consensus motifs, with the arrangement characteristic of the particular set of consensus motifs.

In an alternative embodiment, the methods consist essentially of identifying in the amino acid sequence of the polypeptide, or portion thereof, the consensus bacterial DNA Pol III motifs A, B, and C from a particular set of consensus motifs. In another embodiment, the methods consist essentially of identifying in the amino acid sequence of the polypeptide, or portion thereof, the consensus bacterial DNA Pol III motifs A, B, or A and B, and optionally C from a particular set of consensus motifs. Additional assays may be combined with such sequence-based methods.

In a preferred embodiment, the consensus sequence of motifs A, B, and C for gram negative bacteria dnaE gene product are, respectively, G-[L/M]-[L/V/I]-K-X-D-F-L-G-L-X-X-L-T, [F/W]-X-X-X-X-X-F-X-X-Y-[A/G]-F-N-K-S-H, and S-X-P-D-[F/I]-D-X-D-[F/I], wherein X is any amino acid. The motifs are arranged, from amino terminus to carboxyl terminus, in the order C-A-B. The spacing between motif C and A ranges from about 153 to about 155 amino acids. The spacing between motif A and motif B ranges from about 195 to about 201 amino acids. The spacing from motif C to motif B ranges from about 348 to about 355 amino acids.

In a preferred embodiment, the consensus sequence of motifs A, B, and C for gram positive bacteria dnaE gene product are, respectively, G-[L/V]-[L/V]-K-X-D-[F/I]-L-G-L-[R/K]-X-L-[T/S], [F/Y/W]-X-X-X-X-[R/K]-F-X-X-Y-[A/G]-F-N-[R/K]-X-H, and P-D-I-D-[L/I/V]-D-[F/L/V], wherein X is any amino acid. The motifs are arranged, from amino terminus to carboxyl terminus, in the order C-A-B. The spacing between motif C and A ranges from about 112 to about 150 amino acids. The spacing between motif A and motif B ranges from about 167 to about 190 amino acids. The spacing from motif C to motif B ranges from about 278 to about 339 amino acids.

In a preferred embodiment, the consensus sequence of motifs A, B, and C for gram positive bacteria polC gene product are, respectively, [L/V]-[L/V]-K-X-D-[A/I]-L-G-H-D-X-P-T, [F/Y]-I-X-S-C-X-[R/K]-I-K-Y-[M/L]-F-P-K-A-H, and P-D-I-D-L-D-F-S, wherein X is any amino acid. The motifs are arranged, from amino terminus to carboxyl terminus, in the order C-A-B. The spacing between motif C and A is about 124 amino acids. The spacing between motif A and motif B ranges from about 173 to about 179 amino acids. The spacing from motif C to motif B ranges from about 296 to about 302 amino acids.

In a preferred embodiment, the consensus sequence of motifs A, B, and C for cyanobacteria dnaE gene product are, respectively, G-L-L-K-M-D-F-L-G-L-[R/K]-N-L-T, F-D-Q-M-V-K-F-A-E-Y-C-F-N-K-S-H, P-D-I-D-T-D-F-C. The motifs are arranged, from amino terminus to carboxyl terminus, in the order C-A-B. The spacing between motif C and A is about 100-160 amino acids. The spacing between motif A and motif B about 150-210 amino acids. The spacing from motif C to motif B is about 250-370 amino acids.

In some embodiments, the methods involve the use of PCR and oligonucleotide probes to detect the presence of bacterial DNA Pol III motifs. The primers used are capable of amplifying sequence that comprises bacterial Pol III motifs C-A-B. In one embodiment, the method involves use of a first PCR primer that hybridizes to a nucleotide sequence encoding a bacterial DNA Pol III motif C, and a second PCR primer that corresponds to the nucleotide sequence encoding a bacterial DNA Pol III motif B. PCR is done using the two primers and PCR products are probed with an oligonucleotide probe that hybridizes to a nucleotide sequence encoding a bacterial DNA Pol III motif A, or its complement. In one embodiment, PCR products are combined with a microarray comprising such an oligonucleotide probe that hybridizes to a nucleotide sequence encoding a bacterial DNA Pol III motif A, or its complement. In one embodiment, the methods further comprise determining the spacing of bacterial DNA Pol III motifs C, A, and B from the PCR product. In another embodiment, the size of the PCR product is determined. In another embodiment, primers directed to motifs C and A, or A and B are used, and the size of the PCR product is determined. Alternatively, PCR products may be sequenced.

Exemplary motif spacings in bacterial Pol III subunits include the following:

| | MOTIF C | MOTIF A | MOTIF B |
|---|---|---|---|
| | Distance | Distance | Distance |
| SPECIES | C to A | A to B | C to B |
| Thermophilic Bacteria | | | |
| Thermus thermophilus | 153 AA | 193 AA | 346 AA |
| Thermus aquaticus | 153 AA | 193 AA | 346 AA |
| Aquifex aeolicus | 174 AA | 190 AA | 364 AA |
| Thermotoga neapolitana | 99 AA | 168 AA | 268 AA |
| Thermotoga maritima | 107 AA | 168 AA | 276 AA |
| Cyanobacteria | | | |
| Trichodesmium | 170 AA | 183 AA | 353 AA |
| Thermosynechococcus | 156 AA | 190 AA | 346 AA |
| Synechococcus | 156 AA | 190 AA | 346 AA |
| Prochlorococcus | 156 AA | 190 AA | 346 AA |
| Nostoc | 156 AA | 209 AA | 365 AA |
| Crocosphaera | 156 AA | 209 AA | 365 AA |
| Synechocystis sp. | 156 AA | 209 AA | 365 AA |
| Gloeobacter | 156 AA | 190 AA | 346 AA |
| Anabaena | 156 AA | 209 AA | 359 AA |
| | | | (408 AA) |
| Synechocystis sp. | 156 AA | 209 AA | 365 AA |
| Gram− Bacteria (dnaE) | | | |
| Acinetobacter | (357/41AA) | 189 AA | 347 AA |
| | 158AA | | (436AA) |
| Clostridium | (382AA/41AA) | 200 AA | 350 AA |
| | 150 AA | | (413AA) |
| Deinococcus | (511AA/41AA) | 199 AA | 353 AA |
| | 154AA | | (424AA) |
| E. coli | (362AA/41AA) | 199AA | 351AA |
| | 152AA | | (400AA) |
| Yersinia pestis | 152AA | 199AA | 351AA |
| Wolbachia | 152AA | 187AA | 339AA |
| Helicobacter hepaticus | 164AA | 188AA | 352AA |
| Rickettsia prowazekii | 163AA | 187AA | 350AA |
| Treponema pallidum | 150AA | 190AA | 340AA |
| Borrelia burgdorferi | 149AA | 187AA | 336AA |
| Chlamydophila pneumoniae | 151AA | 188AA | 339AA |
| Methylococcus capsulatus | 152AA | 194AA | 336AA |
| Gram+ Bacteria (PolC) | | | |
| Lactobacillus acidophilus | 123AA | 172AA | 295AA |
| Staphylococcus aureus | 123AA | 171AA | 294AA |
| Mesoplasma florum | 127AA | 172AA | 299AA |
| Ureaplasma parvum | 125AA | 171AA | 297AA |
| Mycoplasma pulmonis | 123AA | 173AA | 296AA |
| Fusobacterium nucleatum | 123AA | 176AA | 299AA |
| Streptococcus pyogenes | 121AA(81) | 177AA | 300AA |
| Gram+ Bacteria (dnaE) | | | |
| Listeria innocua | 151AA(41) | 188AA | 339AA |
| Bacillus halodurans | 151AA(41) | 188AA | 339AA |
| Streptococcus pyogenes | | | |

### Pol III α Mutants

In addition to identifying and describing the functional motifs of bacterial Pol III α active sites, methods for altering the functionality of bacterial Pol III α subunits, and Pol III replicases comprising the same, through amino acid substitution at a variety of positions within motifs A and B are provided herein. The mutations in motifs A and/or B endow the Pol III α mutants with one or more characteristics distinguishing them from Pol III α subunits not having the one or more mutations. Preferred activity alterations include altered primer discrimination and altered dNTP discrimination.

Additional mutations may be introduced into Pol III α subunits to yield α subunits with additional preferred characteristics, such as increased affinity for β subunit. For a detailed description of such additional desirable mutations, see U.S. Provisional Application Ser. No. 60/______, filed Nov. 29, 2005, titled “Two Component DNA Pol (II Replicases with Modified Beta-subunit Binding Motifs, and Uses Thereof”, which is expressly incorporated herein in its entirety by reference.

In one aspect, the invention provides Pol III α mutants of gram negative bacteria, cyanobacteria, and gram positive bacteria. The Pol III α mutants of gram positive bacteria include DnaE and PolC mutants.

Pol III α mutants of the invention are modified, non-naturally occurring Pol III α subunits that have an active site bearing at least one mutation in one or more of motifs A and B, as compared to an unmodified Pol III α subunit. In some cases, these Pol III α variants have a motif A or motif B sequence that falls within the consensus sequence of the respective bacterial type, yet they are non-naturally occurring and correspond to unmodified Pol III α subunits with the exception that they are modified at one or more particular positions within the active site to provide desired functional characteristics that differ from the unmodified Pol III α subunit. Excluded from Pol III α mutants of the invention are mutants having an amino acid sequence identical to a naturally occurring Pol III α subunit known in the prior art.

(i) Pol III Mutants Derived from Gram Negative Bacterial Pol III

Consensus motif A for gram negative bacteria may be represented as X₁-X₂-X₃-X₄-X₅-X₆-X₇-X₈-X₉-X₁₀-X₁₁-X₁₂-X₁₃-X₁₄, wherein X₁ is G; X₂ is [L/M]; X₃ is [L/V/I]; X₄ is K; X₅ is any amino acid; X₆ is D; X₇ is F; X₈ is L; X₉ is G; X₁₀ is L; X₁₁ any amino acid; X₁₂ is any amino acid; X₁₃ is L; and X₁₄ is T.

Exemplary motif A sequences from gram negative bacteria include the following:

| Gram Negative | DnaE Pol III alpha Subunit |
|---|---|
| Bacteria | Motif A Sequence |
| Acinetobacter | GLVKFDFLGLRNLT |
| Agrobacterium | KFMKVDILALGMLT |
| Aquifex aeolicus | GLLKMDFLGLKTLT |
| Bdellovibrio | GLIKFDFLGLKTLT |
| Bordetella | GLVKFDFLGLRNLT |
| Borrelia | GLVKMDFLGLKTLT |
| Candidatus | GLIKFDFLGLRTLT |
| Chlamydia | GMLKVDFLGLKTLT |
| Chlamydophila | GMLKVDFLGLKTLT |
| Chlorobium | GLLKIDYLGLETLA |
| Chlostridium | GLLKMDFLGLRTLT |
| Chromobacterium | GLVKFDFLGLRNLT |
| Thermus thermophilus | GLLKMDFLGLRTLT |
| Corynebacterium | GLLKMDFLGLRNLT |
| Coxiella | GLVKFDFLGLRTLT |
| Deinococcus radiurans | GLIKMDFLGLRTLS |
| Desulfovibrio | GLVKFDFLGLRTMT |
| Thermus aquaticus | GLLKMDFLGLRTLT |
| Escherichia coli | GLVKFDFLGLRTLT |
| Erwinia | GLVKFDFLGLRTLT |
| Geobacter | GLVKFDFLGLKNLT |
| Haemophilus influenca | GLVKFDFLGLRTLT |
| Helicobacter pylorii | GLVKFDFLGLRTLT |
| Leptospira | GLIKMDILGLKNLT |
| Mesorhizobium loti | KILKVDVLALGMLT |
| Mycobacterium bovis | GLVKFDLLGLGMLS |
| Mycobacterium leprae | GLLKMDFLGLRNLT |
| Mycoplasma pulmonis | GFLKIDFLGLKTLS |
| Neisseria | GLVKFDFLGLRNLT |
| Nocardia farcinica | GLVKFDMLGLGMLS |
| Pasteurella | GLVKFDFLGLRTLT |
| Pirellula | GLLKMDFLGLRNLT |
| Porphyromonas | GLIKMDFLGLKTLS |
| Pseudomonas aeruginosa | GLVKFDFLGLRTLT |
| Rhodopseudomonas | GLVKFDFLGLKTLT |
| Rickettsia | GLIKFDFLGLQTLT |
| Salmonella | GLVKFDFLGLRTLT |
| Shewanella | GLVKFDFLGLRTLT |
| Shigella | GLVKFDFLGLRTLT |
| Treponema | GLVKMDFLGLKTLT |
| Tropheryma | GLVKMDFLGLRNLT |
| Wolbachia | GLIKFDFLGLGTLT |
| Wolinella | DLIKFDFLGLKTLT |
| Xylellana | GLVKFDFLGLRTLT |
| Consensus Sequence | G-[L/M]-[L/V/I]-K-X-D-F-L-G- |
| | L-X-X-L-T |

In one aspect, the invention provides Pol III mutants having increased ability to bind labeled dNTPs and incorporate the same into primer extension products.

In one embodiment, such a Pol III α mutant comprises a motif A with a mutation at residue 10, from L (in the unmodified form) to a hydrophobic or aromatic amino acid, preferably selected from I, V, A, C, M, Y, G, and F, with G and A being especially preferred.

In one embodiment, such a Pol III α mutant comprises a motif A with a mutation at residue 11, from the residue extant in the unmodified Pol III α subunit, to a positively charged amino acid or aromatic amino acid or small amino acid, preferably selected from H, Y, F, G, S, A, P, R and H, with R and H being especially preferred.

In one embodiment, such a Pol III α mutant comprises a motif A with a mutation at residue 12, from the residue extant in the unmodified Pol III α subunit, to apolar amino acid or a long chain hydrophobic amino acid, preferably selected from N, S, Q, P, M, C, and L, with S being especially preferred. If X₁₁ is not an amino acid with a small side chain, then X₁₁ is preferably also mutated to yield an amino acid with a small side chain.

In a preferred embodiment, such a Pol III α mutant comprises a motif A having a preferred or especially preferred amino acid at two or more of positions X₁₀, X₁₁, and X₁₂.

In a preferred embodiment, such a Pol III α mutant comprises a motif A having one of these preferred or especially preferred amino acids at one or more of positions X₁₀, X₁₁, and X₁₂, and further comprises an X₈ amino acid that is a hydrophobic or aromatic amino acid, preferably selected from I, V, A, C, M, F.

In a preferred embodiment, such a Pol III α mutant comprises a motif A having a preferred or especially preferred amino acid at one or more of positions X₁₀, X₁₁, and X₁₂, and further comprises an X₉ amino acid that is a small amino acid, preferably selected from A, P, S, and T. In an especially preferred embodiment, X₉ is P.

In a preferred embodiment, such a Pol III α mutant comprises a motif A having a preferred or especially preferred amino acid at one or more of positions X₁₀, X₁₁, and X₁₂, and further comprises an X₁₃ amino acid that is a hydrophobic amino acid, preferably selected from I, V, M, C, and A. In an especially preferred embodiment, X₁₃ is A.

In a preferred embodiment, such a Pol III α mutant comprises a motif A having a preferred or especially preferred amino acid at one or more of positions X₁₀, X₁₁, and X₁₂, and further comprises a preferred or especially preferred amino acid at one or more of positions X₈, X₉, and X₁₃.

Conservative amino acid substitutions may also be incorporated in motif A at other positions, with the exception of X₆. For example, P and S are tolerated at position X₁; V, I, F, A, M, C, and Y are tolerated at position X₂; L, I, F, A, M, C, and Y are tolerated at position X₃; R is tolerated at position X₄; M, V, I, L, C, and Y are tolerated at position X₅; Y, L, I, V, M, C, and A are tolerated at position X₇; S, A, and P are tolerated at position X₁₄.

In a preferred embodiment, such a Pol III α mutant has increased ability to incorporate labeled nucleotides into primer extension products as compared to a Pol III α subunit having (i) the motif A sequence G-L-V-K-F-D-F-L-G-L-[R/K]-T-L-T, the motif B sequence F-D-L-M-E-K-F-A-G-Y-G-F-N-K-S-H, and the motif C sequence P-D-F-D-I-D-F-C.

Consensus motif B for gram negative bacteria may be represented as X₁-X₂-X₃-X₄-X₅-X₆-X₇-X₈-X₉-X₁₀-X₁₁-X₁₂-X₁₃-X₁₄-X₁₅-X₁₆, wherein X₁ is [F/W]; X₂ is any amino acid; X₃ is any amino acid; X₄ is any amino acid; X₅ is any amino acid; X₆ is any amino acid; X₇ is F; X₈ is any amino acid; X₉ is any amino acid; X₁₀ is Y; X₁₁ is [A/G]; X₁₂ is F; X₁₃ is N; X₁₄ is K; X₁₅ is S; X₁₆ is H.

Exemplary gram negative motif B sequences include the following:

| Gram Negative | DnaE Pol III alpha Subunit |
|---|---|
| Bacteria | Motif B Sequence |
| Acinetobacter | FDYMEKFAGYGFNKSH |
| Agrobacterium | FSQLEGFGSYGFPESH |
| Aquifex aeolicus | WEDIEKFASYSFNKSH |
| Bdellovibrio | FDLMYKFADYGFNKSH |
| Bordetella | FDLMEKFAGYGFNKSH |
| Borrelia | FELLKPFSGYGFNKSH |
| Candidatus | FDLMEKFAGYGFNKSH |
| Chlamydia | FDKMEKFASYGFNKSH |
| Chlamydophila | FDKMEKFASYGFNKSH |
| Chlorobium | FDLMAEFAGYGFNKSH |
| Chlostridium | FDSMMDFASYAFNKSH |
| Chromobacterium | FDYMEKFAGYGFNKSH |
| Thermus thermophilus | FDMLEAFANYGFNKSH |
| Corynebacterium | WGTIEPFASYAFNKSH |
| Coxiella | FDLMEKFSGYGFNKSH |
| Deinococcus radiurans | FDMLDAFANYGFNKSH |
| Desulfovibrio | FDLMEKFAEYGFNKSH |
| Thermus aquaticus | FDMLEAFANYGFNKSH |
| Escherichia coli | FDLVEKFAGYGFNKSH |
| Erwinia | FDLVEKFAGYGFNKSH |
| Geobacter | FDLMAKFAEYGFNKSH |
| Haemophilus influenca | FDLVEKFAGYGFNKSH |
| Helicobacter pylorii | WDLIVKFAGYGFNKSH |
| Leptospira | FEQLERFGGYGFNKSH |
| Mesorhizobium loti | FKQIEGFGEYGFPESH |
| Mycobacterium bovis | YEKLEAFANFGFPESH |
| Mycobacterium leprae | WDIILPFADYAFNKSH |
| Mycoplasma pulmonis | YLTIEDFAQYGFNKSH |
| Neisseria | FNYMEKFAGYGFNKSH |
| Nocardia farcinica | YEKLYAFANFGFPESH |
| Pasteurella | FDLVEKFAGYGFNKSH |
| Pirellula | WNLIVKFAGYGFNKSH |
| Porphyromonas | WTDWEKFASYAFNKSH |
| Pseudomonas aeruginosa | FDLVEKFAGYGFNKSH |
| Rhodopseudomonas | FDLLAKFADYGFNKSH |
| Rickettsia | FATVAKFAGYGFNKAH |
| Salmonella | FDLVEKFAGYGFNKSH |
| Shewanella | FDLVEKFAGYGFNKSH |
| Shigella | FDLVEKFAGYGFNKSH |
| Treponema | FEILIPFAGYGFNKSH |
| Tropheryma | WNVLLPFSDYAFNKAH |
| Wolbachia | FDLVAKFAGYGFNKSH |
| Wolinella | FDLIVKFAGYGFNKSH |
| Xylellana | FDLMEKFAGYGFNKSH |
| Consensus Sequence | [F/W]-X-X-X-X-X-F-X-X-Y- |
| | [A/G]-F-N-K-S-H |

In one aspect, the invention provides Pol III α mutants having increased ability to bind labeled dNTPs and incorporate the same into primer extension products.

In one embodiment, such a Pol III α mutant comprises a motif B with a mutation at residue 6, from the residue extant in the unmodified Pol III α subunit, to a charged amino acid or an amino acid with a small side chain or an amino acid with a polar amine, preferably selected from R, E, D, Q, N, A, G, S, T, and P.

In one embodiment, such a Pol III α mutant comprises a motif B with a mutation at residue 7, from F to an uncharged aromatic amino acid, preferably Y or W, with Y being especially preferred.

In one embodiment, such a Pol III α mutant comprises a motif B with a mutation at residue 10, from Y to another aromatic or bulky hydrophobic amino acid, preferably selected from F, H, W, L, M, V, and I, with F, I, and V being especially preferred.

In a preferred embodiment, such a Pol III α mutant comprises a motif B having a preferred or especially preferred amino acid at two or more of positions X₆, X₇, and X₁₀.

In a preferred embodiment, such a Pol III α mutant comprises a motif B having a preferred or especially preferred amino acid at one or more of positions X₆, X₇, and X₁₀, and further comprises an X₅ amino acid that is any amino acid other than P, T, or S.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, V, and I, with G being especially preferred.

In a preferred embodiment, such a Pol III α mutant comprises a motif B having a preferred or especially preferred amino acid at one or more of positions X₆, X₇, and X₁₀, and further comprises an X₉ amino acid that is a small amino acid, a polar amino acid, or a negatively charged amino acid. Preferred are A, S, T, N, Q, E, and D.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, S, P, C, L, and M.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, L, M, C, V, and I, with Y being especially preferred.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, P, and G, with G and S being especially preferred.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are Q, N, P, T, G, and A, with G being especially preferred.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Conservative amino acid substitutions may also be incorporated in motif B at positions 1-4. For example, W, Y, L, I, V, M, and C are tolerated at position X₁; E, K, R, N, Q, T, A, G, and L are tolerated at position X₂; V, I, M, C, and A are tolerated at position X₃; L, 1, V, A, C, Y, and F are tolerated at position X₄; R, K, Y, and F are tolerated at position X₁₆.

In a preferred embodiment, such a Pol III α mutant has increased ability to incorporate labeled nucleotides into primer extension products as compared to a Pol III α subunit having (i) the motif A sequence G-L-V-K-F-D-F-L-G-L-[R/K]-T-L-T, the motif B sequence F-D-L-M-E-K-F-A-G-Y-G-F-N-K-S-H, and the motif C sequence P-D-F-D-I-D-F-C.

In one aspect, the invention provides Pol III α mutants having increased ability to bind ddNTPs and incorporate the same into primer extension products.

In one embodiment, such a Pol III α mutant comprises a motif B with a mutation at residue 6, from the residue extant in the unmodified Pol III α subunit, to a charged amino acid or an amino acid with a small side chain or an amino acid with a polar amine, preferably selected from R, E, D, Q, N, A, G, S, T, and P.

In one embodiment, such a Pol III α mutant comprises a motif B with a mutation at residue 7, from F to an uncharged aromatic amino acid, preferably Y or W, with Y being especially preferred.

In one embodiment, such a Pol III α mutant comprises a motif B with a mutation at residue 10, from Y to another aromatic or bulky hydrophobic amino acid, preferably selected from F, H, W, L, M, V, and I.

In a preferred embodiment, such a Pol III α mutant comprises a motif B having a preferred or especially preferred amino acid at two or more of positions X₆, X₇, and X₁₀.

In a preferred embodiment, such a Pol III α mutant comprises a motif B having a preferred or especially preferred amino acid at one or more of positions X₆, X₇, and X₁₀, and further comprises an X₅ amino acid that is any amino acid other than P, T, or S.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, V, and I, with G being especially preferred.

In a preferred embodiment, such a Pol III α mutant comprises a motif B having a preferred or especially preferred amino acid at one or more of positions X₆, X₇, and X₁₀, and further comprises an X₉ amino acid that is a small amino acid, a polar amino acid, or a negatively charged amino acid. Preferred are A, S, T, N, Q, E, and D.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, S, P, C, L, and M.

In a preferred embodiment, such a Pol III 0 mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, L, M, C, V, and 1, with Y being especially preferred.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, P, and G.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III mutant comprises a motif B having a preferred or especially preferred amino acid at one or more of positions X₆, X₇, and X₁₀, and further comprises an X₁₅ amino acid that is a polar amino acid. Preferred are Q, N, P, T, G, and A, with G being especially preferred.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Conservative amino acid substitutions may also be incorporated in motif B at positions 1-4. For example, W, Y, L, I, V, M, and C are tolerated at position X₁; E, K, R, N, Q, T, A, G, and L are tolerated at position X₂; V, I, M, C, and A are tolerated at position X₃; L, I, V, A, C, Y, and F are tolerated at position X₄; R, K, Y, and F are tolerated at position X₁₆.

In a preferred embodiment, such a Pol III α mutant has increased ability to incorporate ddNTPs into primer extension products as compared to a Pol III α subunit having (i) the motif A sequence G-L-V-K-F-D-F-L-G-L-[R/K]-T-L-T, the motif B sequence F-D-L-M-E-K-F-A-G-Y-G-F-N-K-S-H, and the motif C sequence β-D-F-D-I-D-F-C.

In one aspect, the Invention provides Pol III α mutants altered in their discrimination of RNA and DNA primers. In one embodiment, Pol III α mutants that preferentially replicate RNA-primed template are provided. Such Pol III α mutants preferably bear one or more mutations in motif B. These mutants exhibit a decreased ability to extend DNA primers.

In a preferred embodiment, such a Pol III cc mutant comprises a motif B with a mutation at residue 11, from G to M, C, or L.

In a preferred embodiment, such a Pol III α isoform has increased preference for RNA-primed template as compared to a Pol III α subunit having (I) the motif A sequence G-L-V-K-F-D-F-L-G-L-[R/K]-T-L-T, the motif B sequence F-D-L-M-E-K-F-A-G-Y-G-F-N-K-S-H, and the motif C sequence P-D-F-D-I-D-F-C.

In one embodiment, Pol III α mutants that preferentially replicate DNA-primed template are provided. Such Pol III α mutants preferably bear one or more mutations in motif B. These mutants preferably exhibit a decreased ability to extend RNA primers.

In a preferred embodiment, such a Pol III α mutant comprises a motif B with a mutation at residue 12, from F to Y, and a preferably a second mutation at residue 11, from G to M, C, or L.

In a preferred embodiment, such a Pol III α isoform has increased preference for DNA-primed template as compared to a Pol III α subunit having (i) the motif A sequence G-L-V-K-F-D-F-L-G-L-[R/K]-T-L-T, the motif B sequence F-D-L-M-E-K-F-A-G-Y-G-F-N-K-S-H, and the motif C sequence P-D-F-D-I-D-F-C.

In a preferred embodiment, a Pol III α mutant comprises a motif A and a motif B, which motifs A and B comprise an amino acid sequence described above.

(ii) Pol III Mutants Derived from Gram Positive DnaE

Consensus motif A for gram positive bacteria DnaE may be represented as X₁-X₂-X₃-X₄-X₅-X₆-X₇-X₈-X₉-X₁₀-X₁₁-X₁₂-X₁₃-X₁₄, wherein X₁ is G; X₂ is [L/V]; X₃ is [L/V]; X₄ is K; X₅ is any amino acid; X₆ is D; X₇ is [F/I]; X₈ is L; X₉ is G; X₁₀ is L; X₁₁ is [R/K]; X₁₂ is any amino acid; X₁₃ is L; and X₁₄ is [T/S].

Exemplary DnaE motif A sequences from gram positive bacteria include the following:

| Gram Positive | DnaE Pol III alpha Subunit |
|---|---|
| Bacteria | Motif A Sequence |
| Thermotoga maritima | GVVKIDILGLKTLS |
| Bacillus subtilis | GLLKMDFLGLRNLT |
| Bacillus licheniformis | GLLKMDFLGLRNLT |
| Bacillus cereus | |
| Enterococcus faecalis | GLLKMDFLGLRNLS |
| Streptococcus pyogenes | GLLKMDFLGLRNLT |
| Streptococcus mutans | |
| Staphylococcus aureus | GLLKIDFLGLRNLS |
| Bacillus halodurans | |
| Clostridium | HLVKMDFLGLKTLD |
| acetobutylicum | |
| Thermoanaerobacter | GLLKMDFLGLKNLT |
| Consensus Sequence | G-[L/V]-[L/V]-K-X-D-[F/I]- |
| | L-G-L-[R/K]-X-L-[T/S] |

In one aspect, the invention provides Pot III (mutants having increased ability to bind labeled dNTPs and incorporate the same into primer extension products.

In one embodiment, such a Pol III α mutant comprises a motif A with a mutation at residue X₁₀, from L (in the unmodified form) to a hydrophobic or aromatic amino acid, preferably selected from I, V, A, C, M, Y, and F.

In one embodiment, such a Pol III α mutant comprises a motif A with a mutation at residue X₁₁, from [R/K] to a positively charged amino acid or an aromatic amino acid or a small amino acid. Preferred are H, Y, F, G, S, A, and P.

In one embodiment, such a Pol III α mutant comprises a motif A with a mutation at residue X₁₂, from the residue extant in the unmodified Pol III α subunit, to a polar amino acid or a long chain hydrophobic amino acid. Preferred are T, S, Q, P, M, C, and L. If X₁₂ in the mutant is a polar or long hydrophobic amino acid, and X₁₁ is not an amino acid with a small side chain, then X₁₁ is mutated to an amino acid with a small side chain.

In one embodiment, such a Pol III α mutant comprises a motif A with a mutation at residue X₄, from K to a positively charged amino acid. Preferred are R and H.

In a preferred embodiment, such a Pol III α mutant comprises a motif A having a preferred or especially preferred amino acid at two or more of positions X₁₀, X₁₁, and X₁₂, and X₄.

In a preferred embodiment, such a Pol III α mutant comprises a motif A having one of these preferred or especially preferred amino acids at one or more of positions X₁₀, X₁₁, X₁₂, and X₄, and further comprises an X₈ amino acid that is hydrophobic or aromatic, preferably selected from I, V, A, C, M, F.

In a preferred embodiment, such a Pol III α mutant comprises a motif A having a preferred or especially preferred amino acid at one or more of positions X₁₀, X₁₁, and X₁₂, and X₄, and further comprises an X₉ amino acid that is a small amino acid, preferably selected from A, P, S, and T.

In a preferred embodiment, such a Pol III α mutant comprises a motif A having a preferred or especially preferred amino acid at one or more of positions X₁₀, X₁₁, and X₁₂, and X₄, and further comprises an X₁₃ amino acid that is a hydrophobic amino acid, preferably selected from I, V, M, C, and A.

In a preferred embodiment, such a Pol III α mutant comprises a motif A having a preferred or especially preferred amino acid at one or more of positions X₁₀, X₁₁, and X₁₂, and X₄, and further comprises a preferred or especially preferred amino acid at one or more of positions X₈, X₉, and X₁₃.

Additional conservative amino acid substitutions are tolerated. Notably, substitution at position 6 is not tolerated. For example, V, I, F, A, M, C, and Y are tolerated at positions X₂ and X₃; F, V, L, I, C, and Y are tolerated at position X₅; Y, L, I, V, M, C, and A are tolerated at position X₇; P is tolerated at position X₁₄.

In a preferred embodiment, such a Pol III α isoform has increased ability to incorporate labeled nucleotides into primer extension products as compared to a Pol III α subunit having the motif A sequence G-L-L-K-M-D-F-L-G-L-[R/K]-N-L-[T/S], the motif B sequence Y-D-L-I-[L/V]-K-F-A-N-Y-G-F-N-R-S-H, and the motif C sequence β-D-F-D-L-D-F-S.

Consensus motif B for gram positive bacteria DnaE may be represented as X₁-X₂-X₃-X₄-X₅-X₆-X₇-X₈-X₉-X₁₀-X₁₁-X₁₂-X₁₃-X₁₄-X₁₅-X₁₆, wherein X₁ is [F/Y/W], X₂ is any amino acid, X₃ is any amino acid, X₄ is any amino acid, X₅ is any amino acid, X₃ is [R/K], X₇ is F, X₈ is any amino acid, X₉ is any amino acid, X₁₀ is Y, X₁₁ is [A/G], X₁₂ is F, X₁₃ is N, X₁₄ is [R/K], X₁₅ is any amino acid, X₁₆ is H.

Exemplary DnaE motif B sequences from gram positive bacteria include the following:

| Gram Positive | DnaE Pol III alpha Subunit |
|---|---|
| Bacteria | Motif B Sequence |
| Thermotoga maritima | LEILLNFSSYAFNKSH |
| Bacillus subtilis | YDLIVKFANYGFNRSH |
| Bacillus licheniformis | YDLIVKFANYGFNRSH |
| Bacillus cereus | YDLIVRFANYGFNRSH |
| Enterococcus faecalis | YDYIERFANYGFNRSH |
| Streptococcus pyogenes | FKRMEKFAGYGFNRSH |
| Streptococcus mutans | FARMAKFAGYGFNRSH |
| Staphylococcus aureus | FDLILKFADYGFPRAH |
| Bacillus halourans | YELIVRFANYGFNKSH |
| Clostridium | WKLLLKQATYSFNKGH |
| acetobutylicum | |
| Thermoanaerobacter | |
| Consensus Sequence | [F/Y/W]-X-X-X-X-[R/K]-F-X- |
| | X-Y-[A/G]-F-N-[R/K]-X-H |

In one aspect, the invention provides Pol III α mutants having increased ability to bind labeled dNTPs and incorporate the same into primer extension products.

In one embodiment, such a Pol III α mutant comprises a motif B with a mutation at residue X₆, from [R/K] to a positively charged amino acid, preferably R or H.

In one embodiment, such a Pol III α mutant comprises a motif B with a mutation at residue X₇, from F to an uncharged aromatic amino acid, preferably Y or W.

In one embodiment, such a Pol III α mutant comprises a motif B with a mutation at residue X₁₀, from Y to another aromatic or bulky hydrophobic amino acid, preferably selected from F, H, W, L, M, V, and I.

In a preferred embodiment, such a Pol III α mutant comprises a motif B having a preferred or especially preferred amino acid at two or more of positions X₆, X₇, and X₁₀.

In a preferred embodiment, such a Pol III α mutant comprises a motif B having a preferred or especially preferred amino acid at one or more of positions X₆, X₇, and X₁₀, and further comprises an X₅ amino acid that is hydrophobic or aromatic. Preferred are L, I, A, C, M, F, W, and Y.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, V, and I.

In a preferred embodiment, such a Pol III α mutant comprises a motif B having a preferred or especially preferred amino acid at one or more of positions X₆, X₇, and X₁₀, and further comprises an X₉ amino acid that is a small amino acid, a polar amino acid, or a negatively charged amino acid. Preferred are A, S, T, N, Q, G, and D.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, S, P, G, L, C, and M.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, L, M, C, V, and I.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, P, and G.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are Q, N, P, T, G, A.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Additional conservative amino acid substitutions are tolerated. Notably, substitution at position 6 is not tolerated. For example, V, I, F, A, M, C, and Y are tolerated at positions X₂ and X₃; F, V, L, I, C, and Y are tolerated at position X₅; Y, L, I, V, M, C, and A are tolerated at position X₇; P is tolerated at position X₁₄.

In a preferred embodiment, such a Pol III α isoform has increased ability to incorporate labeled nucleotides into primer extension products as compared to a Pol III α subunit having the motif A sequence G-L-L-K-M-D-F-L-G-L-[R/K]-N-L-[T/S], the motif B sequence Y-D-L-I-[L/V]-K-F-A-N-Y-G-F-N-R-S-H, and the motif C sequence P-D-F-D-L-D-F-S.

In one aspect, the invention provides Pol III α mutants having increased ability to bind ddNTPs and incorporate the same into primer extension products.

In one embodiment, such a Pol III α mutant comprises a motif B with a mutation at residue X₆, from [R/K] to a positively charged amino acid, preferably R or H.

In one embodiment, such a Pol III α mutant comprises a motif B with a mutation at residue 7, from F to an uncharged aromatic amino acid, preferably Y or W.

In one embodiment, such a Pol III α mutant comprises a motif B with a mutation at residue 10, from Y to another aromatic or bulky hydrophobic amino acid, preferably selected from F, H, W, L, M, V, and I.

In a preferred embodiment, such a Pol III α mutant comprises a motif B having a preferred or especially preferred amino acid at two or more of positions X₆, X₇, and X₁₀.

In a preferred embodiment, such a Pol III α mutant comprises a motif B having a preferred or especially preferred amino acid at one or more of positions X₆, X₇, and X₁₀, and further comprises an X₅ amino acid that is hydrophobic or aromatic. Preferred are L, I, A, C, M, F, W, and Y.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, V, and I.

In a preferred embodiment, such a Pol III α mutant comprises a motif B having a preferred or especially preferred amino acid at one or more of positions X₆, X₇, and X₁₀, and further comprises an X₉ amino acid that is a small amino acid, a polar amino acid, or a negatively charged amino acid. Preferred are A, S, T, N, Q, G, and D.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, S, P, G, L, C, and M.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, L, M, C, V, and 1.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, P, and G.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are Q, N, P, T, G, A.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Additional conservative amino acid substitutions are tolerated. Notably, substitution at position 6 is not tolerated. For example, V, I, F, A, M, C, and Y are tolerated at positions X₂ and X₃; F, V, L, I, C, and Y are tolerated at position X₅; Y, L, I, V, M, C, and A are tolerated at position X₇; P is tolerated at position X₁₄.

In a preferred embodiment, such a Pol III α isoform has increased ability to incorporate ddNTPs into primer extension products as compared to a Pol III α subunit having the motif A sequence G-L-L-K-M-D-F-L-G-L-[R/K]-N-L-[T/S], the motif B sequence Y-D-L-[L/V]-K-F-A-N-Y-G-F-N-R-S-H, and the motif C sequence β-D-F-D-L-D-F-S.

In one aspect, the invention provides Pol III α mutants altered in their discrimination of RNA and DNA primers. In one embodiment, Pol III α mutants that preferentially replicate RNA-primed template are provided. Such Pol III α mutants preferably bear one or more mutations in motif B. These mutants exhibit a decreased ability to extend DNA primers.

In a preferred embodiment, such a Pol III α mutant comprises a motif B with a mutation at residue 11, from [A/G] to M, C, or L.

In a preferred embodiment, such a Pol III α isoform has increased preference for RNA-primed template as compared to a Pol III α subunit having the motif A sequence G-L-L-K-M-D-F-L-G-L-[R/K]-N-L-[T/S], the motif B sequence Y-D-L-I-[LN]-K-F-A-N-Y-G-F-N-R-S-H, and the motif C sequence P-D-F-D-L-D-F-S.

In one embodiment, Pol III α mutants that preferentially replicate DNA-primed template are provided. Such Pol III α mutants preferably bear one or more mutations in motif B. These mutants exhibit a decreased ability to extend RNA primers.

In a preferred embodiment, such a Pol III α mutant comprises a motif B with a mutation at residue 12, from F to Y, and a preferably a second mutation at residue 11, from [A/G] to M, C, or L.

In a preferred embodiment, such a Pol III α isoform has increased preference for DNA-primed template as compared to a Pol III c subunit having the motif A sequence G-L-L-K-M-D-F-L-G-L-[R/K]N-L-[T/S], the motif B sequence Y-D-L-I-[LN]-K-F-A-N-Y-G-F-N-R-S-H, and the motif C sequence P-D-F-D-L-D-F-S.

In a preferred embodiment, a Pol III α mutant comprises a motif A and a motif B, which motifs A and B comprise an amino acid sequence described above.

(iii) Pol III Mutants Derived from Gram Positive PolC

Consensus motif A for gram positive bacteria PolC may be represented as X₁-X₂-X₃-X₄-X₅-X₆-X₇-X₈-X₉-X₁₀-X₁₁-X₁₂-X₁₃, wherein X₁ is [L/V]; X₂ is [L/V]; X₃ is K; X₄ is any amino acid; X₅ D; X₆ is [A/I]; X₇ is L; X₈ is G; X₉ is H; X₁₀ is D; X₁₁ is any amino acid; X₁₂ is P; X₁₃ is T.

Exemplary PolC motif A sequences from gram positive bacteria include the following:

| Gram Positive | PolC Pol III alpha Subunit |
|---|---|
| Bacteria | Motif A Sequence |
| Thermotoga maritima | LVKIDALGHDDPT |
| Bacillus subtilis | LLKLDILGHDDPT |
| Bacillus licheniformis | LLKLDILGHDDPT |
| Bacillus cereus | LLKLDILGHDDPT |
| Enterococcus faecalis | ILKLDILGHDDPT |
| Streptococcus pyogenes | VLKLDILGHDDPT |
| Staphylococcus epidermis | VLKLDILGHDDPT |
| Staphylococcus aureus | VLKLDILGHDDPT |
| Streptococcus agalactiae | VLKLDILGHDDPT |
| Bacillus halodurans | LLKLDILGHDDPT |
| Listeria monocytogenes | VLKLDILGHDDPT |
| Listeria innocua | VLKLDILGHDDPT |
| Clostridium perfringens | LLKLDILGHDDPT |
| Lactococcus lactis | ILKLDILGHDDPT |
| Oceanobacillus iheyensis | LLKLDILGHDDPT |
| Onion yellows | LFKLDILGHDDPM |
| phytoplasma | |
| Thermoanaerobacter | LLKLDILGHDDPT |
| Ureaplasma parvum | LLKFDILGHDNPT |
| Consensus Sequence | [L/V]-[L/V]-K-X-D-[A/I]-L- |
| | G-H-D-X-P-T |

In one aspect, the invention provides Pol III mutants having increased ability to bind labeled dNTPs and incorporate the same into primer extension products.

In one embodiment, such a Pol III α mutant comprises a motif A with a mutation at residue X₉ from H to an aromatic amino acid, preferably selected from Y, F, and W.

In one embodiment, such a Pol III α mutant comprises a motif A with a mutation at residue X₁₀, from D to a negatively charged amino acid or an amino acid with a polar amine, preferably selected from E, Q, and N.

In one embodiment, such a Pol III α mutant comprises a motif A with a mutation at residue X₁₁, from the extant amino acid to a negatively charged amino acid or an amino acid with a polar amine, preferably selected from E, Q, and N.

In one embodiment, such a Pol III α mutant comprises a motif A with a mutation at residue X₃, from K to a positively charged amino acid, preferably R or H.

In a preferred embodiment, such a Pol III α mutant comprises a motif A having a preferred or especially preferred amino acid at two or more of positions X₉, X₁₀, and X₁₁, and X₃.

In a preferred embodiment, such a Pol III α mutant comprises a motif A having one of these preferred or especially preferred amino acids at one or more of positions X₉, X₁₀, and X₁₁, and X₃, and further comprises an X₇ amino acid that is hydrophobic or aromatic, preferably selected from 1, V, A, C, M, F.

In a preferred embodiment, such a Pol III α mutant comprises a motif A having a preferred or especially preferred amino acid at one or more of positions X₉, X₁₀, and X₁₁, and X₃, and further comprises an X₈ amino acid that is a small amino acid, preferably selected from A, P, S, and T.

In a preferred embodiment, such a Pol III α mutant comprises a motif A having a preferred or especially preferred amino acid at one or more of positions X₉, X₁₀, and X₁₁, and X₃, and further comprises an X₁₂ amino acid that is a small amino acid or a polar amino acid, preferably selected from S, T, G, N, and Q.

In a preferred embodiment, such a Pol III α mutant comprises a motif A having a preferred or especially preferred amino acid at one or more of positions X₉, X₁₀, and X₁₁, and X₃, and further comprises a preferred or especially preferred amino acid at one or more of positions X₇, X₈, and X₁₂.

Additional conservative substitutions are tolerated. Notably, substitution at position 6 is not tolerated. For example, V, I, F, A, M, C, and Y are tolerated at positions X₂ and X₃; F, V, I, L, C, and Y are tolerated at position X₅; F, L, I, V, M, C, A are tolerated at position X₇; S, A, P, G are tolerated at position X₁₄.

In a preferred embodiment, such a Pol III α isoform has increased ability to incorporate labeled nucleotides into primer extension products as compared to a Pol III α subunit having the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

Consensus motif B for gram positive bacteria PolC may be represented as X₁-X₂-X₃-X₄-X₅-X₆-X₇-X₈-X₉-X₁₀-X₁₁-X₁₂-X₁₃-X₁₄-X₁₅-X₁₆, wherein X₁ is [F/Y]; X₂ is I; X₃ is any amino acid; X₄ is S; X₅ is C; X₆ is any amino acid; X₇ is [R/K]; X₈ is I; X₉ is K; X₁₀ is Y; X₁₁ is [M/L]; X₁₂ is F; X₁₃ is P; X₁₄ is K; X₁₅ is A; X₁₆ is H.

Exemplary PolC motif B sequences from gram positive bacteria include the following:

| Gram Positive | PolC Pol III alpha Subunit |
|---|---|
| Bacteria | Motif B Sequence |
| Thermotoga maritima | FIESCKRIKYLFPKAH |
| Bacillus subtilis | YIDSCKKIKYMFPKAH |
| Bacillus licheniformis | YIDSCKKIKYMFPKAH |
| Bacillus cereus | YIDSCKKIKYMFPKAH |
| Enterococcus faecalis | YIDSCSKIKYMFPKAH |
| Streptococcus pyogenes | YIESCGKIKYMFPKAH |
| Staphylococcus epidermis | YLDSCRKIKYMFPKAH |
| Staphylococcus aureus | YLDSCLKIKYMFPKAH |
| Streptococcus agalactiae | YIESCGKIKYMFPKAH |
| Bacillus halodurans | YIGSCLKIKYMFPKAH |
| Listeria monocytogenes | YIESCKKIKYMFPKAH |
| Listeria innocua | YIESCKKIKYMFPKAH |
| Clostridium perfringens | YIESCKRIKYMFPKGH |
| Lactococcus lactis | YIESCSKIKYMFPKAH |
| Oceanobacillus iheyensis | YIESCKKTKYMFPKAH |
| Onion yellows | YIDSAAKIKYLFPKAH |
| phytoplasma | |
| Thermoanaerobacter | FIQSCQKIKYMFPKAH |
| Ureaplasma parvum | YIESANKIKYMFPKAH |
| Consensus Sequence | [F/Y]-I-X-S-C-X-[R/K]-I-K- |
| | Y-[M/L]-F-P-K-A-H |

In one aspect, the invention provides Pol III α mutants having increased ability to bind labeled dNTPs and incorporate the same into primer extension products.

In one embodiment, such a Pol III α mutant comprises a motif B with a mutation at residue X₇, from [R/K] to a positively charged amino acid, preferably R or H.

In one embodiment, such a Pol III α mutant comprises a motif B with a mutation at residue X₉, from K to a positively charged amino acid, preferably R or H.

In one embodiment, such a Pol III α mutant comprises a motif B with a mutation at residue X₁₀, from Y to another aromatic amino acid, preferably F, H, or W, with F and W being especially preferred.

In a preferred embodiment, such a Pol III α mutant comprises a motif B having a preferred or especially preferred amino acid at two or more of positions X₇, X₉, and X₁₀.

In a preferred embodiment, such a Pol III α mutant comprises a motif B having a preferred or especially preferred amino acid at one or more of positions X₇, X₉, and X₁₀, and further comprises an X₅ amino acid that is hydrophobic or aromatic. Preferred are L, I, A, C, M, F, W, and Y.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, A, V, and I, with G, S, and A being especially preferred.

In a preferred embodiment, such a Pol III α mutant comprises a motif B having a preferred or especially preferred amino acid at one or more of positions X₆, X₇, and X₁₀, and further comprises an X₉ amino acid that is a small hydrophobic or a bulky hydrophobic amino acid and not a charged or aromatic amino acid. Preferred are G, S, L, C, M, V, and I, with R, S, and G being especially preferred.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, G, L, C, and M, with A and G being especially preferred.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, V, and I.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, N, and G, with G and S being especially preferred.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are S, Q, N, P, T, G, with G and S being especially preferred.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Additional conservative substitutions are tolerated. For example, W, Y, L, I, V, M, and C are tolerated at position X₁; L, V, C, M, G, A, are tolerated at position X₂; D, Q, N are tolerated at position X₃; T, N, Q, E, D are tolerated at position X₄.

In a preferred embodiment, such a Pol III α isoform has increased ability to incorporate labeled nucleotides into primer extension products as compared to a Pol III α subunit having the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

In one aspect, the invention provides Pol III α mutants having increased ability to bind ddNTPs and incorporate the same into primer extension products.

In one embodiment, such a Pol III α mutant comprises a motif B with a mutation at residue X₇, from [R/K] to a positively charged amino acid, preferably R or H.

In one embodiment, such a Pol III α mutant comprises a motif B with a mutation at residue X₉, from K to a positively charged amino acid, preferably R or H.

In one embodiment, such a Pol III α mutant comprises a motif B with a mutation at residue X₁₀, from Y to another aromatic amino acid, preferably F, H, or W, with F and W being especially preferred.

In a preferred embodiment, such a Pol III mutant comprises a motif B having a preferred or especially preferred amino acid at two or more of positions X₇, X₉, and X₁₀.

In a preferred embodiment, such a Pol III α mutant comprises a motif B having a preferred or especially preferred amino acid at one or more of positions X₇, X₉, and X₁₀, and further comprises an X₅ amino acid that is hydrophobic or aromatic. Preferred are L, I, A, C, M, F, W, and Y.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, A, V, and I, with A, L, G and S being especially preferred.

In a preferred embodiment, such a Pol III α mutant comprises a motif B having a preferred or especially preferred amino acid at one or more of positions X₆, X₇, and X₁₀, and further comprises an X₉ amino acid that is a small hydrophobic or a bulky hydrophobic amino acid and not a charged or aromatic amino acid. Preferred are G, S, L, C, M, V, and I, with R, S, and G being especially preferred.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, G, L, C, and M, with A, G, and L being especially preferred.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, V, and I, with Y being especially preferred.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, N, and G, with G and S being especially preferred.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are S, Q, N, P, T, G.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₆, X₉, X₁₁, X₁₂, X₁₃, X₁₄, and X₁₅.

Additional conservative substitutions are tolerated. For example, W, Y, L, I, V, M, and C are tolerated at position X₁; L, V, C, M, G, A, are tolerated at position X₂; D, Q, N are tolerated at position X₃; T, N, Q, E, D are tolerated at position X₄.

In a preferred embodiment, such a Pol III α isoform has increased ability to incorporate ddNTPs into primer extension products as compared to a Pol III subunit having the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

In one aspect, the invention provides Pol III α mutants altered in their discrimination of RNA and DNA primers. In one embodiment, Pol III α mutants that preferentially replicate RNA-primed template are provided. Such Pol III α mutants preferably bear one or more mutations in motif B. These mutants exhibit a decreased ability to extend DNA primers.

In one embodiment, such a Pol III α mutant comprises a motif B with a mutation at residue 11, from [M/L] to C.

In a preferred embodiment, such a Pol III α isoform has increased preference for RNA-primed template as compared to a Pol III α subunit having the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

In one embodiment, Pol III α mutants that preferentially replicate DNA-primed template are provided. Such Pol III α mutants preferably bear one or more mutations in motif B. These mutants exhibit a decreased ability to extend RNA primers.

In a preferred embodiment, such a Pol III α mutant comprises a motif B with a mutation at residue 12, from F to Y, and optionally a second mutation at residue 11, from [M/L] to C.

In a preferred embodiment, such a Pol III α isoform has increased preference for DNA-primed template as compared to a Pol III α subunit having the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

In a preferred embodiment, a Pol III α mutant comprises a motif A and a motif B, which motifs A and B comprise an amino acid sequence described above.

(iv) Pol III Mutants Derived from Cyanobacteria Pol III

Consensus motif A for cyanobacteria DnaE may be represented as X₁-X₂-X₃-X₄-X₅-X₆-X₇-X₈-X₉-X₁₀-X₁₁-X₁₂-X₁₃-X₁₄, wherein X₁ is G; X₂ is L; X₃ is L; X₄ is K; X₅ is M; X₆ is D; X₇ is F; X₈ is L; X₉ is G; X₁₀ is L; X₁₁ is [R/K]; X₁₂ is N; X₁₃ is L; X₁₄ is T.

Exemplary motif A sequences from cyanobacteria include the following:

| | DnaE Pol III alpha Subunit |
|---|---|
| Cyanobacteria | Motif A Sequence |
| Trichodesmium | GLLKMDFLGLKNLT |
| Thermosynechococcus | GLLKMDFLGLKNLT |
| Synechococcus | GLLKMDFLGLRNLT |
| Prochlorococcus | GLLKMDFLGLKNLT |
| Nostoc | GLLKMDFLGLRNLT |
| Crocosphaera | GLLKMDFLGLRNLT |
| Synechocystis sp. | GLLKMDFLGLKNLT |
| Gloeobacter | GLLKMDFLGLRNLT |
| Anabaena | GLLKMDFLGLKNLT |
| Synechocystis sp. | GLLKMDFLGLKNLT |
| Consensus Sequence | GLLKMDFLGL^{R}/_{K}NLT |

In one aspect, the invention provides Pol III α mutants having increased ability to bind labeled dNTPs and incorporate the same into primer extension products.

In one embodiment, such a Pol III α mutant comprises a motif A with a mutation at residue X₁₀ from L to an aromatic amino acid or a hydrophobic amino acid, preferably selected from I, V, A, C, M, Y, and F.

In one embodiment, such a Pol III α mutant comprises a motif A with a mutation at residue X₁₀ from [R/K] to an aromatic amino acid or a small amino acid or an positively charged amino acid, preferably selected from H, Y, F, G, S, A, and P.

In one embodiment, such a Pol III α mutant comprises a motif A with a mutation at residue X₁₂ from N to a polar amino acid or a long chain hydrophobic amino acid, preferably selected from T, S, Q, P, M, C, and L. If X₁₁ is not a small amino acid, position X₁₁ is preferably also mutated to yield a small amino acid.

In one embodiment, such a Pol III α mutant comprises a motif A with a mutation at residue X₄ from K to a positively charged amino acid, preferably R or H.

In one embodiment, such a Pol III α mutant comprises a motif A having a preferred or especially preferred amino acid at two or more of positions X₁₀, X₁₁, and X₁₂, and X₄.

In a preferred embodiment, such a Pol III α mutant comprises a motif A having a preferred or especially preferred amino acid at one or more of positions X₁₀, X₁₁, and X₁₂, and X₄, and further comprises an X₈ amino acid that is an aromatic or hydrophobic amino acid, preferably selected from I, V, A, C, M, and F.

In a preferred embodiment, such a Pol III α mutant comprises a motif A having a preferred or especially preferred amino acid at one or more of positions X₁₀, X₁₁, and X₁₂, and X₄, and further comprises an X₉ amino acid that is a small amino acid, preferably selected from A, P, S, and T.

In a preferred embodiment, such a Pol III α mutant comprises a motif A having a preferred or especially preferred amino acid at one or more of positions X₁₀, X₁₁, and X₁₂, and X₄, and further comprises an X₁₃ amino acid that is a hydrophobic amino acid, preferably selected from I, V, M, C, and A.

In a preferred embodiment, such a Pol III α mutant comprises a motif A having a preferred or especially preferred amino acid at one or more of positions X₁₀, X₁₁, and X₁₂, and X₄, and further comprises a preferred or especially preferred amino acid at one or more of positions X₈, X₉, and X₁₃.

Additional conservative substitutions are tolerated. Notably, substitution at position X₆ is not tolerated. For example, I, F, A, M, C, Y are tolerated at positions X₂ and X₃; F, I, V, L, C, Y are tolerated at position X₅; Y, L, I, V, M, C, A are tolerated at position X₇; S, A, P are tolerated at position X₁₄.

In a preferred embodiment, such a Pol III α isoform has increased ability to incorporate labeled nucleotides into primer extension products as compared to a Pol III α subunit having the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C.

Consensus motif B for cyanobacteria DnaE may be represented as X₁-X₂-X₃-X₄-X₅-X₆-X₇-X₈-X₉-X₁₀-X₁₁-X₁₂-X₁₃-X₁₄-X₁₅-X₁₆, wherein X₁ is F; X₂ is D; X₃ is Q; X₄ is M; X₅ is V; X₆ is K; X₇ is F; X₈ is A; X₉ is E; X₁₀ is Y; X₁₁ is C; X₁₂ is F; X₁₃ is N; X₁₄ is K; X₁₅ is S; X₁₆ is H.

Exemplary motif B sequences from cyanobacteria include the following:

| | DnaE Pol III alpha Subunit |
|---|---|
| Cyanobacteria | Motif B Sequence |
| Trichodesmium | FEQMIKFAEYCFNKSH |
| Thermosynechococcus | FKQMLDFAEYCFNKSH |
| Synechococcus | FDQMVLFAEYCFNKSH |
| Prochlorococcus | FDQMVLFAEYCFNKSH |
| Nostoc | FEQMLKFAEYCFNKSH |
| Crocosphaera | FEQMIKFAEYCFNKSH |
| Synechocystis sp. | FDQMVKFAEYCFNKSH |
| Gloeobacter | FEQMVVFAEYCFNKSH |
| Anabaena | FEDMLKFAEYCFNKSH |
| Synechocystis sp. | FDQMVKFAEYC????? |
| Consensus Sequence | FDQMVKFAEYCFNKSH |

In one aspect, the invention provides Pol III mutants having increased ability to bind labeled dNTPs and incorporate the same into primer extension products.

In one embodiment, such a Pol III α mutant comprises a motif B with a mutation at residue X₆, from K to a small amino acid or a charged amino acid or a polar amino acid, preferably selected from K, E, D, Q, N, A, G, S, T, and P.

In one embodiment, such a Pol III α mutant comprises a motif B with a mutation at residue X₇, from F to an uncharged aromatic amino acid, preferably Y or W.

In one embodiment, such a Pol III α mutant comprises a motif B with a mutation at residue X₁₀, from Y to another aromatic amino acid or bulky hydrophobic amino acid, preferably selected from F, H, W, L, M, V, and I.

In a preferred embodiment, such a Pol III α mutant comprises a motif B having a preferred or especially preferred amino acid at two or more of positions X₆, X₇, and X₁₀.

In a preferred embodiment, such a Pol III α mutant comprises a motif B having a preferred or especially preferred amino acid at one or more of positions X₆, X₇, and X₁₀, and further comprises an X₅ amino acid that is hydrophobic or aromatic. Preferred are L, I, A, C, M, F, W, and Y.

In a preferred embodiment, such a Pol III α mutant comprises a motif B having a preferred or especially preferred amino acid at one or more of positions X₆, X₇, and X₁₀, and further comprises an X₈ amino acid that is a small hydrophobic or bulky hydrophobic amino acid and not a charged or aromatic amino acid. Preferred are G, S, L, C, M, V, and I.

In a preferred embodiment, such a Pol III α mutant comprises a motif B having a preferred or especially preferred amino acid at one or more of positions X₆, X₇, and X₁₀, and further comprises an X₉ amino acid that is a small hydrophobic amino acid or a polar amino acid or a negatively charged amino acid. Preferred are A, S, T, N, Q, G, and D.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, S, P, G, L, and M.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, L, M, C, V, and I.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, P, and G.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are Q, N, P, T, G, and A.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Additional conservative substitutions are tolerated. For example, W, Y, L, I, V, M, C are tolerated at position X₁; E, Q, N are tolerated at position X₂; D, E, N are tolerated at position X₃; L, I, V, A, C, Y, F are tolerated at position X₄.

In a preferred embodiment, such a Pol III α isoform has increased ability to incorporate labeled nucleotides into primer extension products as compared to a Pol III α subunit having the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C.

In one aspect, the invention provides Pol III α mutants having increased ability to bind ddNTPs and incorporate the same into primer extension products.

In one embodiment, such a Pol III α mutant comprises a motif B with a mutation at residue X₆, from K to a small amino acid or a charged amino acid or a polar amino acid, preferably selected from K, E, D, Q, N, A, G, S, T, and P.

In one embodiment, such a Pol III α mutant comprises a motif B with a mutation at residue X₇, from F to an uncharged aromatic amino acid, preferably Y or W.

In one embodiment, such a Pol III α mutant comprises a motif B with a mutation at residue X₁₀, from Y to another aromatic amino acid or bulky hydrophobic amino acid, preferably selected from F, H, W, L, M, V, and I.

In a preferred embodiment, such a Pol III α mutant comprises a motif B having a preferred or especially preferred amino acid at two or more of positions X₆, X₇, and X₁₀.

In a preferred embodiment, such a Pol III α mutant comprises a motif B having a preferred or especially preferred amino acid at one or more of positions X₆, X₇, and X₁₀, and further comprises an X₅ amino acid that is hydrophobic or aromatic. Preferred are L, I, A, C, M, F, W, and Y.

In a preferred embodiment, such a Pol III α mutant comprises a motif B having a preferred or especially preferred amino acid at one or more of positions X₆, X₇, and X₁₀, and further comprises an X₆ amino acid that is a small hydrophobic or bulky hydrophobic amino acid and not a charged or aromatic amino acid. Preferred are G, S, L, C, M, V, and I.

In a preferred embodiment, such a Pol III α mutant comprises a motif B having a preferred or especially preferred amino acid at one or more of positions X₆, X₇, and X₁₀, and further comprises an X₉ amino acid that is a small hydrophobic amino acid or a polar amino acid or a negatively charged amino acid. Preferred are A, S, T, N, Q, G, and D.

In a preferred embodiment, such a Pol III α mutant comprises a motif B having a preferred or especially preferred amino acid at one or more of positions X₆, X₇, and X₁₀, and further comprises an X₁₁ amino acid that is a small amino acid or a non-branched hydrophobic amino acid, which is not an aromatic amino acid. Preferred are A, S, P, G, L, and M.

In a preferred embodiment, such a Pol III α mutant comprises a motif B having a preferred or especially preferred amino acid at one or more of positions X₆, X₇, and X₁₀, and further comprises an X₁₂ amino acid that is a non-charged aromatic amino acid or a bulky hydrophobic amino acid. Preferred are Y, W, L, M, C, V, and I.

In a preferred embodiment, such a Pol III α mutant comprises a motif B having a preferred or especially preferred amino acid at one or more of positions X₆, X₇, and X₁₀, and further comprises an X₁₃ amino acid that is a polar amino acid. Preferred are Q, S, T, P, and G.

In a preferred embodiment, such a Pol III α mutant comprises a motif B having a preferred or especially preferred amino acid at one or more of positions X₆, X₇, and X₁₀, and further comprises an X₁₄ amino acid that is positively charged. Preferred are R and H.

In a preferred embodiment, such a Pol III α mutant comprises a motif B having a preferred or especially preferred amino acid at one or more of positions X₆, X₇, and X₁₀, and further comprises an X₁₅ amino acid that is a polar amino acid. Preferred are Q, N, P, T, G, and A.

In a preferred embodiment, such a Pol III α mutant comprises a motif B having a preferred or especially preferred amino acid at one or more of positions X₆, X₇, and X₁₀, and further comprises a preferred or especially preferred amino acid at one or more of positions X₅, X₈, X₉, X₁₁, X₁₂, X₁₃, X₁₄, and X₁₅.

Additional conservative substitutions are tolerated. For example, W, Y, L, I, V, M, C are tolerated at position X₁; E, Q, N are tolerated at position X₂; D, E, N are tolerated at position X₃; L, I, V, A, C, Y, F are tolerated at position X₄.

In a preferred embodiment, such a Pol III α isoform has increased ability to incorporate ddNTPs into primer extension products as compared to a Pol III α subunit having the motif A sequence G-L-L-K-M-D-F-L-G-L-[R/K]-N-L-T, the motif B sequence F-D-Q-M-V-K-F-A-E-Y-C-F-N-K-S-H, and the motif C sequence F-D-Q-D-T-D-F-C.

In one aspect, the invention provides Pol III α mutants altered in their discrimination of RNA and DNA primers. In one embodiment, Pol III α mutants that preferentially replicate RNA-primed template are provided. Such Pol III α mutants preferably bear one or more mutations in motif B. These mutants exhibit a decreased ability to extend DNA primers.

In one embodiment, such a Pol III α mutant comprises a motif B with a mutation at residue 11, from C to [M/L].

In a preferred embodiment, such a Pol III α isoform has increased preference for RNA-primed template as compared to a Pol III α subunit having the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C.

In one embodiment, Pol III α mutants that preferentially replicate DNA-primed template are provided. Such Pol III α mutants preferably bear one or more mutations in motif B. These mutants exhibit a decreased ability to extend RNA primers.

In a preferred embodiment, such a Pol III α mutant comprises a motif B with a mutation at residue 12, from F to Y, and optionally a second mutation at residue 11, from C to [M/L].

In a preferred embodiment, such a Pol III α isoform has increased preference for DNA-primed template as compared to a Pol III α subunit having the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C.

In a preferred embodiment, a Pol III α mutant comprises a motif A and a motif B, which motifs A and B comprise an amino acid sequence described above.

Pol III α Subunit Isoforms with Preferred Characteristics

In another aspect, the invention provides Pol III α isoforms having preferred characteristics, such as preferred primer discrimination or preferred nucleotide discrimination activity. These Pol III a isoforms may be naturally occurring isoforms, or Pol III α mutants. Regardless, based on the nexus between motif sequence and activity disclosed herein, these isoforms are, for the first time, recognized on the basis of motif sequence as having the ability to bind ddNTPs or labeled nucleotides and incorporate the same in primer extension products, or as having preferred primer discrimination activity, thus making them useful in particular methods described herein in place of Pol III α mutants, as described herein.

The amino acid sequences of motifs A, B, and C in such Pol III α isoforms fall within the motif sequences described above for Pol III α mutants.

In a preferred embodiment, such a Pol III α isoform has increased ability to incorporate ddNTPs or labeled nucleotides into primer extension products as compared to a Pol III α subunit having (i) the motif A sequence G-L-V-K-F-D-F-L-L-L-[R/K]-T-L-T, the motif B sequence F-D-L-M-E-K-F-A-G-Y-G-F-N-K-S-H, and the motif C sequence P-D-F-D-I-D-F-C; (ii) the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C; (iii) the motif A sequence G-L-L-K-M-D-F-L-G-L-[R/K]-N-L-[T/S], the motif B sequence Y-D-L-I-[L/V]-K-F-A-N-Y-G-F-N-R-S-H, and the motif C sequence P-D-F-D-L-D-F-S; or (iv) the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

In another preferred embodiment, such a Pol III α isoform has increased preference for RNA-primed template as compared to a Pol III α subunit having (i) the motif A sequence G-L-V-K-F-D-F-L-G-L-[R/K]-T-L-T, the motif B sequence F-D-L-M-E-K-F-A-G-Y-G-F-N-K-S-H, and the motif C sequence P-D-F-D-I-D-F-C; (ii) the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C; (iii) the motif A sequence G-L-L-K-M-D-F-L-G-L-[R/K]-N-L-[T/S], the motif B sequence Y-D-L-I-[L/V]-K-F-A-N-Y-G-F-N-R-S-H, and the motif C sequence P-D-F-D-L-D-F-S; or (iv) the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

In another preferred embodiment, such a Pol III α isoform has increased preference for DNA-primed template as compared to a-Pol III α subunit having (i) the motif A sequence G-L-V-K-F-D-F-L-G-L-[R/K]-T-L-T, the motif B sequence F-D-L-M-E-K-F-A-G-Y-G-F-N-K-S-H, and the motif C sequence P-D-F-D-I-D-F-C; (ii) the motif A sequence G-L-L-K-M-D-F-L-G-L-[R/K]-N-L-T, the motif B sequence F-D-Q-M-V-K-F-A-E-Y-C-F-N-K-S-H, and the motif C sequence P-D-I-D-T-D-F-C; (iii) the motif A sequence G-L-L-K-M-D-F-L-G-L-[R/K]-N-L-[T/S], the motif B sequence Y-D-L-I-[L/V]-K-F-A-N-Y-G-F-N-R-S-H, and the motif C sequence P-D-F-D-L-D-F-S; or (iv) the motif A sequence N-L-L-K-L-D-I-L-G-H-D-D-P-T, the motif B sequence Y-I-E-S-C-K-K-I-K-Y-M-F-P-K-A-H, and the motif C sequence P-D-I-D-L-N-F-S.

### Single Component and Two Component Pol III Replicases

In one aspect, the invention provides modified Pol III replicases that are single component Pol III replicases. In another aspect, the invention provides modified Pol III replicases that are two component Pol III replicases. For a detailed description of single component and two component Pol III replicases, see WO2005/113810, International Application Serial No. PCT/US2005/011978, which is expressly incorporated herein in its entirety by reference. A brief description of single component and two component Pol III replicases follows.

Contrary to the findings of previous reports, bacterial dnaE encoded and polC encoded α subunits can independently function alone and/or in combination with a processivity clamp component of a Pol III as a minimal functional Pol III replicase under appropriate conditions in vitro. Such single component and two component Pol III replicases lack a Pol III clamp loader.

Further, some dnaE encoded α subunits, characterized by dnaE encoded α subunits of gram negative bacteria, and more particularly by those of non-mesophilic bacteria, possess intrinsic zinc-dependent 3′-5′ exonuclease activity, and functional Pol III replicase activity in the absence of a clamp loader. Also, polC encoded α subunits, characterized by polC encoded α subunits of gram positive bacteria, and more particularly by those of non-mesophilic bacteria, possess functional Pol III replicase activity in the absence of a clamp loader. Such α subunits are useful in one component and two component Pol III replicases. Preferred for use are α subunits derived from extremeophiles. Especially preferred for use are α subunits derived from thermophiles.

Surprisingly, the presence and function of a clamp loader component is not required for proper functioning of single component and two component Pol III replicases in vitro. Also surprising is the finding that single component and two component Pol III replicases can replicate a primed ssDNA template molecule with high speed and processivity in vitro without the assistance of an initiation complex formed by the clamp loader. Despite the absence of a clamp loader, and in the case of single component Pol III replicases, the absence of a processivity clamp, the extension rates of the minimal functional Pol III replicases of the invention are at least 6 to 8 times faster than those of any type A or B repair DNA polymerase currently used for DNA sequencing, amplification, quantification, labeling and reverse transcription, such as Taq DNA polymerase I (type A), Klenow Fragment of E. coli DNA polymerase I (type A), T7 DNA polymerase (type A), Bst DNA polymerase I (type A), phi29 DNA polymerase (type B), Pfu DNA polymerase (type B), Tli DNA polymerase (type B) or KOD DNA polymerase (type B).

Additionally, single component and two component Pol III replicases derived from thermophilic organisms exhibit sufficient thermostability under appropriate conditions to sustain repetitive DNA replication a temperature-cycled mode leading to the amplification of double stranded DNA molecules in vitro.

The single component Pol III replicases may consist of a single subunit or multiple subunits. The single component Pol III replicases consist essentially of a first component of a minimal Pol III, which first component comprises an α subunit, and lack a clamp loader. In some preferred embodiments, the first component consists essentially of an α subunit. In other preferred embodiments, the first component comprises one or more additional subunits of the core polymerase complex of a Pol Ill. Single component Pol III replicases of the invention thus include an α subunit and lack a γ and/or τ subunit. A variety of α subunits may be used in the single component Pol III replicases of the invention.

Thermostable single component Pol III replicases are preferably derived from a thermophilic bacterium or thermophilic cyanobacterium. In a preferred embodiment, the thermophilic bacterium is selected from the group consisting of the genera Thermus, Aquifex, Thermotoga, Thermocridis, Deinococcus, Methanobacterium, Hydrogenobacter, Geobacillus, Thermosynchecoccus and Thermoanaerobacter. Especially preferred are single component and two component Pol IIIs derived from Aquifex aeolicus, Aquifex pyogenes, Thermus thermophilus, Thermus aquaticus, Thermotoga neapolitana and Thermotoga maritima.

The α subunit of a minimal functional Pol III replicase herein is encoded by a bacterial polC or dnaE gene, wherein the dnaE encoded α subunit possesses intrinsic zinc-dependent 3′-5′ exonuclease activity.

In an especially preferred embodiment, the bacterial dnaE or polC encoded α subunits are derived from a bacterium or cyanobacterium selected from the group consisting of Aquifex aeolicus, Thermus thermophilus, Deinococcus radiurans, Thermus aquaticus, Thermotoga maritima, Thermoanaerobacter, Geobacillus stearothermophilus, Thermus flavus, Thermus ruber, Thermus brockianus, Thermotoga neapolitana and other species of the Thermotoga genus, Methanobacterium thermoautotrophicum, and species from the genera Thermocridis, Hydrogenobacter, Thermosynchecoccus, and mutants of these species. In one embodiment, a single component Pol III includes a θ subunit and/or an ε subunit, which subunits are preferably from the same species as the α subunit of the single component Pol III.

### Two-Component Polymerases

The two component Pol III replicases disclosed herein consist essentially of a first component and a second component, wherein the first component is a single component Pol III replicase, and the second component comprises a processivity clamp. In a preferred embodiment, the second component consists essentially of a processivity clamp. In preferred embodiments, the processivity clamp comprises a Pol III β subunit. In some preferred embodiments, the processivity clamp consists essentially of a Pol III β subunit. The two component Pol III replicases of the invention also lack a clamp loader component. In some embodiments, a two component Pol III comprises more than one first component, which may be the same or different.

In a preferred embodiment, the first component of a two component DNA polymerase comprises an α subunit encoded by a bacterial dnaE or PolC gene, preferably of a thermophilic bacterium. Examples of α subunits are found, for example, in U.S. Pat. No. 6,238,905, issued May 29, 2001; U.S. patent application Ser. No. 09/642,218, filed Aug. 18, 2000; U.S. patent application Ser. No. 09/716,964, filed Nov. 21, 2000; U.S. patent application Ser. No. 09/151,888, filed Sep. 11, 1998; and U.S. patent application Ser. No. 09/818,780, filed Mar. 28, 2001, each of which is expressly incorporated herein by reference. The first component of the two component DNA polymerase optionally comprises an E subunit encoded by a bacterial dnaQ gene, preferably of a thermophilic bacterium. Examples of E subunits are found, for example, in U.S. patent application Ser. No. 09/642,218, filed Aug. 18, 2000; U.S. patent application Ser. No. 09/716,964, filed Nov. 21, 2000; U.S. patent application Ser. No. 09/151,888, filed Sep. 11, 1998; and U.S. patent application Ser. No. 09/818,780, filed Mar. 28, 2001. Additionally, the second component of the two component DNA polymerase comprises a β subunit encoded by a bacterial dnaN gene, preferably of a thermophilic bacterium. Examples of β subunits are found, for example, in U.S. patent application Ser. No. 09/642,218, filed Aug. 18, 2000; U.S. patent application Ser. No. 09/716,964, filed Nov. 21, 2000; U.S. patent application Ser. No. 09/151,888, filed Sep. 11, 1998, and U.S. patent application Ser. No. 09/818,780, filed Mar. 28, 2001.

In some preferred embodiments, the first component of the two component polymerase possesses 3′→5′ exonuclease activity, which in some embodiments is conferred by the α subunit and in other embodiments is conferred by an ε subunit. The component conferring 3′→5′ exonuclease activity to the two component polymerase may vary with pH and Zn²⁺ concentration of the reaction buffer used.

The two component polymerases of the present invention may be derived, for example, from the bacteria Acinetobacter, Agrobacterium, Aquifex aeolicus, Bdellovibrio, Bordetella, Borrelia, Candidatus, Chiamydia, Chlamydophila, Chlorobium, Chlostridium, Chromobacterium, Thermus thermophilus, Corynebacterium, Coxiella, Deinococcus radiurans, Desulfovibrio, Thermus aquaticus, Escherichia coli, Erwinia, Geobacter, Haemophilus influenca, Helicobacter pylori, Leptospira, Mesorhizobium loti, Mycobacterium bovis, Mycobacterium leprae, Mycoplasma pulmones, Neisseria, Nocardia farcinica, Pasteurella, Pirellula, Porphyromonas, Pseudomonas aeruginosa, Rhodopseudomonas, Rickettsia, Salmonella, Shewanella, Shigella, Treponema, Tropheryma, Wolbachia, Wolinella, Xylellana, Thermotoga maritima, Bacillus subtilis, Bacillus licheniformis, Bacillus cereus, Enterococcus faecalis, Streptococcus pyogenes, Streptococcus mutans, Staphylococcus aureus, Bacillus halourans, Clostridium acetobutylicum, Thermoanaerobacter, Thermococcus litoralis, Pyrococcus furiosus, Pyrococcus woosii, other species of the Pyrococcus genus, Bacillus stearothermophilus, Sulfolobus acidocaldarius, Thermoplasma acidophilum, Thermusflavus, Thermus ruber, Thermus brockianus, Thermotoga neaPolitana and other species of the Thermotoga genus, Methanobacterium thermoautotrophicum, and mutants of these species.

In some preferred embodiments, the two component polymerase is a thermostable polymerase.

In a preferred embodiment, the first and second components of the two component polymerase are derived from a thermophilic bacterium. In a preferred embodiment, the thermophilic bacterium is from a genera selected from the group consisting of Thermus, Aquifex, Thermotoga, Thermocridis, Hydrogenobacter, Thermosynchecoccus and Thermoanaerobacter. Especially preferred are two component poymerases derived from Aquifex aeolicus, Aquifex pyogenes, Thermus thermophilus, Thermus aquaticus, Thermotoga neapolitana and Thermotoga maritima.

### Nucleic Acid Replication

In one aspect, the invention provides methods for replicating a nucleic acid molecule, comprising subjecting the nucleic acid molecule to a replication reaction in a replication reaction mixture comprising a modified Pol III replicase.

“Nucleic acid replication” is a process by which a template nucleic acid molecule is replicated in whole or in part. Thus, the product of a nucleic acid replication reaction can be completely or partially complementary to the template nucleic acid molecule it is replicating. Nucleic acid replication is done by extending a primer hybridized to the template nucleic acid in the 5′-3′ direction, incorporating nucleotides complementary to the bases of the template nucleic acid at each position in the extension product. The primer may be, for example, a synthetic oligonucleotide that hybridizes to a region of a single stranded DNA template. The primer may also be, for example, a portion of a single stranded DNA template that is complementary to a second region of the single stranded DNA template and can self-prime. Included within the scope of nucleic acid replication reactions are isothermal replication reactions, sequencing reactions, amplification reactions, thermocycling amplification reactions, PCR, fast PCR, and long range PCR.

The nucleic acid replicated in a nucleic acid replication reaction is preferably DNA, and replication preferably involves the DNA-dependent DNA polymerase activity of a modified Pol III replicase disclosed herein.

In a preferred embodiment, a replication reaction mixture comprises a zwitterionic buffer, comprising a combination of a weak organic acid, having a pK between about 7.0-8.5 (e.g., HEPES, DIPSO, TMPS, HEPBS, HEPPSO, TRICINE, POPSO, MOBS, TAPSO, and TES) and a weak organic base, having a pK between about 6.8-8.5 (e.g., Tris, Bis-Tris-propane, imidazol, TMNO, 4-methyl imidazol, and diethanolamine), wherein the pH of the buffer is set by titration with organic base between about pH 7.5-8.9, and wherein the concentration of the organic acid is between about 10-40 mM, more preferably between about 20-30 mM.

In an especially preferred embodiment, a replication reaction mixture and modified Pol III replicase combination is selected from the following combinations: (i) HEPES-Bis-Tris-Propane (20 mM, pH 7.5) with a modified Pol III replicase comprising a modified dnaE encoded α subunit from the genus Thermus, preferably from the species Thermus thermophilus; and (ii) TAPS-Tris (20 mM, pH 8.7) with a modified Pol III replicase comprising a modified dnaE encoded α subunit from the genus Aquifex, preferably from the species Aquifex aeolicus.

In a preferred embodiment, a nucleic acid replication reaction mixture comprises one or more ions selected from the group consisting of Zn²⁺, Mg²⁺, K⁺, and NH₄²⁺, which are included for optimum activity of the modified Pol III replicase in the reaction mixture. The ions are preferably titrated in preliminary assays to determine the optimum concentrations for optimum activity of the modified Pol III replicase in the reaction mixture. In a particularly preferred embodiment, the nucleic acid replication reaction mixture lacks Ca²⁺ ion.

In some preferred embodiments, the nucleic acid replication reaction mixture includes potassium ions. Potassium ions are preferably titrated initially to determine the optimal concentration for the modified Pol III replicase being used. Generally, the K⁺ concentration of the replication reaction mixture is preferably between 0 and about 100 mM, more preferably between about 5-25 mM. Potassium ion is preferably provided in the form of KCl, K₂SO₄, or potassium acetate. The particular counter anion provided with K⁺ can impact the activity of the modified Pol III replicase, and preliminary assays are preferably done in order to determine which counter anion best suits the particular modified Pol III replicase for the particular replication reaction. In general, sulfate or chloride counter anion is preferably used with a modified Pol III replicase derived from Aquifex aeolicus, with sulfate being preferred over chloride. Additionally, potassium ion is not preferred for use in a replication reaction mixture with a modified Pol III replicase derived from Thermus thermophilus.

In some preferred embodiments, the nucleic acid replication reaction mixture includes ammonium ions. Ammonium ions are preferably titrated initially to determine the optimal concentration for the modified Pol III replicase being used. Generally, the NH₄²⁺ concentration of the replication reaction mixture is preferably between 0 and about 15 mM. Ammonium ion is preferably provided in the form of ammonium sulfate. Ammonium ions are preferably included in a replication reaction mixture with a modified Pol III replicase derived from Aquifex aeolicus. Additionally, ammonium ion is not preferred for use in a replication reaction mixture with a modified Pol III replicase derived from Thermus thermophilus.

In some preferred embodiments, the replication reaction mixture includes zinc ions. Zinc ions are preferably titrated initially to determine the optimal concentration for the modified Pol III replicase being used. Generally, the Zn²⁺ concentration in a replication reaction mixture is preferably between 0 and about 50 μM, more preferably between about 5-15 μM. Zinc ion is preferably provided in the form of a salt selected from the group consisting of ZnSO₄, ZnCl₂ and zinc acetate. The particular counter anion provided with Zn²⁺ can impact the activity of the modified Pol III replicase, and preliminary assays are preferably done in order to determine which counterion best suits the particular modified Pol III replicase for the particular replication reaction. In general, chloride or acetate counter anions are preferably used in a replication reaction mixture with a modified Pol III replicase derived from Thermus thermophilus, and sulfate counter anions are preferably used in a replication reaction mixture with a modified Pol III replicase derived from Aquifex aeolicus.

In general, Zn²⁺ is not preferred for use in sequencing reaction mixtures, as it can increase the 3′-5′ exonuclease activity of a number of α subunits (e.g., Thermus thermophilus dnaE). The impact of Zn²⁺ on the 3′-5′ exonuclease activity of any particular Pol III replicase, and its impact on sequencing reactions using the same, may be assessed using standard exonuclease activity assays that are well known in the art.

In some preferred embodiments, the replication reaction mixture includes magnesium ions. Magnesium ions are preferably titrated initially to determine the optimal concentration for the modified Pol III replicase being used. Generally, the Mg²⁺ concentration in a replication reaction mixture is preferably between 0 and about 15 mM, more preferably between about 4-10 mM. In general, isothermal nucleic acid replication reactions, including nucleic acid sequencing reactions, are more accommodating of Mg²⁺ concentrations at the higher end of the preferred concentration range. Magnesium ion is preferably provided in the form of a salt selected from the group consisting of MgCl₂, MgSO₄, and magnesium acetate. The particular counter anion provided with Mg²⁺ can impact the activity of the modified Pol III replicase, and preliminary assays are preferably done in order to determine which counterion best suits the particular modified Pol III replicase for the particular replication reaction. In general, acetate or chloride counter anions are preferably used with a modified Pol III replicase derived from Thermus thermophilus, with acetate being preferred over chloride. Additionally, sulfate counter anions are preferably used with a modified Pol III replicase derived from Aquifex aeolicus.

In an especially preferred embodiment, a replication reaction mixture for use with a modified Pol III replicase derived from Aquifex aeolicus comprises TAPS-Tris (20 mM, pH8.7), 25 mM K₂SO₄, 10 mM NH₄(OAc)₂, and 10 mM MgSO₄.

In another especially preferred embodiment, a replication reaction mixture for use with a modified Pol III replicase derived from Thermus thermophilus comprises HEPES-Bis-Tris-Propane (20 mM, pH7.5), and 10 mM Mg(OAc)₂.

In one embodiment, a helicase is included in a replication reaction in order to lower the denaturation temperature required to provide single stranded nucleic acid template for replication.

In one embodiment, a replication reaction mixture provided herein lacks ATP.

In one embodiment, a replication reaction mixture provided herein lacks SSB, wherein SSB, if present in the replication reaction mixture, would reduce the DNA polymerase activity of the particular modified Pol III replicase used in the replication reaction mixture. In a preferred embodiment, a replication reaction mixture comprising a modified Pol III replicase, which modified Pol III replicase comprises an α subunit encoded by Streptococcus pyogenes polC lacks SSB.

In nucleic acid replication reactions herein, wherein the modified Pol III replicase used is derived from a thermophilic bacterium, the reaction mixture preferably has a pH from about 7.2-8.9. In some preferred embodiments, the reaction mixture has a Zn²⁺ concentration between 0 and about 50 μM, more preferably between about 5-15 μM. In some preferred embodiments, the reaction mixture has a Mg²⁺ concentration between 0 and about 15 mM, more preferably between about 4-10 mM. In some preferred embodiments, the reaction mixture has a K⁺ concentration between 0 and about 100 mM, more preferably between about 5-25 mM. In some preferred embodiments, the reaction mixture has an NH₄²⁺ concentration between 0 and about 12 mM, more preferably between about 5-12 mM. In some preferred embodiments, the reaction mixture has a combination of these cations in their preferred concentration ranges.

In nucleic acid replication reactions herein, the temperature at which primer extension is done is preferably between about 55° C.-72° C., more preferably between about 60° C.-68° C.

In a preferred embodiment, the temperature at which primer annealing and primer extension are done in a thermocycling amplification reaction is between about 55° C.-72° C., more preferably between about 60° C.-68° C., more preferably between about 60° C.-65° C., though the optimum temperature will be determined by primer length, base content, degree of primer complementarity to template, and other factors, as is well known in the art.

In a preferred embodiment, the temperature at which denaturation is done in a thermocycling amplification reaction is between about 86° C.-95° C., more preferably between 87° C.-93° C., with temperatures at the lower end of the range being preferred for use in combination with thermocycling amplification reaction mixtures that include DNA destabilizers, as disclosed herein. Preferred thermocycling amplification methods include polymerase chain reactions involving from about 10 to about 100 cycles, more preferably from about 25 to about 50 cycles, and peak temperatures of from about 86° C.-95° C., more preferably 87°-93° C., with temperatures at the lower end of the range being preferred for use in combination with PCR reaction mixtures that include DNA destabilizers, as disclosed herein.

### Nucleic Acid Amplification

In one aspect, the invention provides methods for amplifying a nucleic acid molecule, comprising subjecting the nucleic acid molecule to an amplification reaction in an amplification reaction mixture comprising a modified Pol III replicase disclosed herein. Preferably, the amplification reaction is done in an amplification reaction tube described herein.

Nucleic acid molecules may be amplified according to any of the literature-described manual or automated amplification methods. As used herein “amplification” refers to any in vitro method for increasing the number of copies of a desired nucleotide sequence. The nucleic acid amplified is preferably DNA, and amplification preferably involves the DNA-dependent DNA polymerase activity of a modified Pol III replicase described herein.

In one embodiment, nucleic acid amplification results in the incorporation of nucleotides into a DNA molecule or primer, thereby forming a new DNA molecule complementary to a nucleic acid template. The formed DNA molecule and its template can be used as templates to synthesize additional DNA molecules. As used herein, one amplification reaction may consist of many rounds of DNA replication. DNA amplification reactions include, for example, polymerase chain reactions (“PCR”). One PCR reaction may consist of 10 to 100 “cycles” of denaturation and synthesis of a DNA molecule. Such methods include, but are not limited to, PCR (as described in U.S. Pat. Nos. 4,683,195 and 4,683,202, which are hereby incorporated by reference), Strand Displacement Amplification (“SDA”) (as described in U.S. Pat. No. 5,455,166, which is hereby incorporated by reference), and Nucleic Acid Sequence-Based Amplification (“NASBA”) (as described in U.S. Pat. No. 5,409,818, which is hereby incorporated by reference). For example, amplification may be achieved by a rolling circle replication system which may even use a helicase for enhanced efficiency in DNA melting with reduced heat (see Yuzhakou et al., Cell 86:877-886 (1996) and Mok et al., J. Biol. Chem. 262:16558-16565 (1987), which are hereby incorporated by reference).

In a preferred embodiment, the temperature at which denaturation is done in a thermocycling amplification reaction is between about 86° C.-95° C., more preferably between 87° C.-93° C., with temperatures at the lower end of the range being preferred for use in combination with thermocycling amplification reaction mixtures that include DNA destabilizers, as disclosed herein. Preferred thermocycling amplification methods include polymerase chain reactions involving from about 10 to about 100 cycles, more preferably from about 25 to about 50 cycles, and peak temperatures of from about 86° C.-93° C., more preferably 87° C.-93° C., with temperatures at the lower end of the range being preferred for use in combination with PCR reaction mixtures that include DNA destabilizers, as disclosed herein. In an especially preferred embodiment, the thermostable modified Pol III replicase comprises a dnaE α subunit, preferably of the genus Thermus or Aquifex, preferably of the species Thermus thermophilus, Thermus aquaticus, or Aquifex aeolicus.

In a preferred embodiment, the amplification reaction mixture used in an amplification reaction involving one or more high temperature denaturation steps further comprises stabilizers that contribute to the thermostability or the modified Pol III replicase, as described and exemplified more fully herein.

In a preferred embodiment, an amplification mixture provided herein lacks SSB, wherein SSB, if present in the replication reaction mixture, would inhibit the DNA polymerase activity of the particular modified Pol III replicase used in the replication reaction mixture.

In a preferred embodiment, a PCR reaction is done using a modified Pol III replicase with appropriate stabilizers to produce, in exponential quantities relative to the number of reaction steps involved, at least one target nucleic acid sequence, given (a) that the ends of the target sequence are known in sufficient detail that oligonucleotide primers can be synthesized which will hybridize to them and (b) that a small amount of the target sequence is available to initiate the chain reaction. The product of the chain reaction will be a discrete nucleic acid duplex with termini corresponding to the ends of the specific primers employed.

Any source of nucleic acid, in purified or nonpurified form, can be utilized as the starting nucleic acid, if it contains or is thought to contain the target nucleic acid sequence desired. Thus, the process may employ, for example, DNA or RNA, including messenger RNA, which DNA or RNA may be single stranded or double stranded. In addition, a DNA-RNA hybrid which contains one strand of each may be utilized. A mixture of any of these nucleic acids may also be employed, or the nucleic acids produced from a previous amplification reaction using the same or different primers may be so utilized. The nucleic acid amplified is preferably DNA. The target nucleic acid sequence to be amplified may be only a fraction of a larger molecule or can be present initially as a discrete molecule, so that the target sequence constitutes the entire nucleic acid. It is not necessary that the target sequence to be amplified be present initially in a pure form; it may be a minor fraction of a complex mixture, such as a portion of the β-globin gene contained in whole human DNA or a portion of nucleic acid sequence due to a particular microorganism which organism might constitute only a very minor fraction of a particular biological sample. The starting nucleic acid may contain more than one desired target nucleic acid sequence which may be the same or different. Therefore, the method is useful not only for producing large amounts of one target nucleic acid sequence, but also for amplifying simultaneously multiple target nucleic acid sequences located on the same or different nucleic acid molecules.

The nucleic acid(s) may be contained from any source and include plasmids and cloned DNA or RNA, as well as DNA or RNA from any source, including bacteria, yeast, viruses, and higher organisms such as plants or animals. DNA or RNA may be extracted from, for example, blood or other fluid, or tissue material such as corionic villi or amniotic cells by a variety of techniques such as that described by Maniatis et al., Molecular Cloning: A Laboratory Manual, (New York: Cold Spring Harbor Laboratory) pp 280-281 (1982).

Any specific (i.e., target) nucleic acid sequence can be produced by the present methods. It is only necessary that a sufficient number of bases at both ends of the target sequence be known in sufficient detail so that two oligonucleotide primers can be prepared which will hybridize to different strands of the desired sequence and at relative positions along the sequence such that an extension product synthesized from one primer, when it is separated from its template (complement), can serve as a template for extension of the other primer into a nucleic acid of defined length. The greater the knowledge about the bases at both ends of the sequence, the greater the specificity of the primers for the target nucleic acid sequence, and, thus, the greater the efficiency of the process. It will be understood that the word primer as used hereinafter may refer to more than one primer, particularly in the case where there is some ambiguity in the information regarding the terminal sequence(s) of the fragment to be amplified. For instance, in the case where a nucleic acid sequence is inferred from protein sequence information a collection of primers containing sequences representing all possible codon variations based on degeneracy of the genetic code can be used for each strand. One primer from this collection will be homologous with the end of the desired sequence to be amplified.

In some alternative embodiments, random primers, preferably hexamers, are used to amplify a template nucleic acid molecule. In such embodiments, the exact sequence amplified is not predetermined.

In addition, it will be appreciated by one of skill in the art that one-sided amplification using a single primer can be done.

Oligonucleotide primers may be prepared using any suitable method, such as, for example, the phosphotriester and phosphodiester methods or automated embodiments thereof. In one such automated embodiment diethylophosphoramidites are used as starting materials and may be synthesized as described by Beaucage et al., Tetrahedron Letters, 22:1859-1862 (1981), which is hereby incorporated by reference. One method for synthesizing oligonucleotides on a modified solid support is described in U.S. Pat. No. 4,458,006, which is hereby incorporated by reference. It is also possible to use a primer which has been isolated from a biological source (such as a restriction endonuclease digest).

Preferred primers have a length of from about 15-100, more preferably about 20-50, most preferably about 20-40 bases. Notably, preferred primers for use herein are longer than those preferred for Pol I polymerases.

The target nucleic acid sequence is amplified by using the nucleic acid containing that sequence as a template. If the nucleic acid contains two strands, it is necessary to separate the strands of the nucleic acid before it can be used as the template, either as a separate step or simultaneously with the synthesis of the primer extension products. This strand separation can be accomplished by any suitable denaturing method including physical, chemical, or enzymatic means. One physical method of separating the strands of the nucleic acid involves heating the nucleic acid until it is completely (>99%) denatured. Typical heat denaturation may involve temperatures ranging from about 80° C. to 105° C., preferably about 90° C. to about 98° C., still more preferably 93° C. to 94° C., for times ranging from about 1 to 10 minutes. Strand separation may also be induced by an enzyme from the class of enzymes known as helicases or the enzyme RecA, which has helicase activity and is known to denature DNA. The reaction conditions suitable for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), and techniques for using RecA are reviewed in C. Radding, Ann. Rev. Genetics, 16:405-37 (1982), which is hereby incorporated by reference. Preferred helicases for use in the present invention are encoded by dnaB.

If the original nucleic acid containing the sequence to be amplified is single stranded, its complement is synthesized by adding oligonucleotide primers thereto. If an appropriate single primer is added, a primer extension product is synthesized in the presence of the primer, a modified Pol III replicase, and the four nucleotides described below. The product will be partially complementary to the single-stranded nucleic acid and will hybridize with the nucleic acid strand to form a duplex of unequal length strands that may then be separated into single strands, as described above, to produce two single separated complementary strands.

If the original nucleic acid constitutes the sequence to be amplified, the primer extension product(s) produced will be completely complementary to the strands of the original nucleic acid and will hybridize therewith to form a duplex of equal length strands to be separated into single-stranded molecules.

When the complementary strands of the nucleic acid are separated, whether the nucleic acid was originally double or single stranded, the strands are ready to be used as a template for the synthesis of additional nucleic acid strands. This synthesis can be performed using any suitable method. Generally, it occurs in a buffered aqueous solution. In some preferred embodiments, the buffer pH is about 8.5 to 8.9, notably different from the preferred pH ranges of Pol I enzymes. Preferably, a molar excess (for cloned nucleic acid, usually about 1000:1 primer:template, and for genomic nucleic acid, usually about 10⁶:1 primer:template) of the two oligonucleotide primers is added to the buffer containing the separated template strands. It is understood, however, that the amount of complementary strand may not be known if the process herein is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

Nucleoside triphosphates, preferably dATP, dCTP, dGTP, dTTP, and/or dUTP are also added to the synthesis mixture in adequate amounts.

The newly synthesized strand and its complementary nucleic acid strand form a double-stranded molecule which is used in the succeeding steps of the process. In the next step, the strands of the double-stranded molecule are separated using any of the procedures described above to provide single-stranded molecules.

New nucleic acid is synthesized on the single-stranded molecules. Additional polymerase, nucleotides, and primers may be added if necessary for the reaction to proceed under the conditions described above. Again, the synthesis will be initiated at one end of the oligonucleotide primers and will proceed along the single strands of the template to produce additional nucleic acids.

The steps of strand separation and extension product synthesis can be repeated as often as needed to produce the desired quantity of the specific nucleic acid sequence. The amount of the specific nucleic acid sequence produced will increase in an exponential fashion.

When it is desired to produce more than one specific nucleic acid sequence from the first nucleic acid or mixture of nucleic acids, the appropriate number of different oligonucleotide primers are utilized. For example, if two different specific nucleic acid sequences are to be produced, four primers are utilized. Two of the primers are specific for one of the specific nucleic acid sequences and the other two primers are specific for the second specific nucleic acid sequence. In this manner, each of the two different specific sequences can be produced exponentially by the present process. Of course in instances where terminal sequences of different template nucleic acid sequences are the same, primer sequences will be identical to each other and complementary to the template terminal sequences.

Additionally, as mentioned above, in an alternative embodiment, random primers, preferably hexamers, are used to amplify a template nucleic acid molecule.

Additionally, one-sided amplification using a single primer may be done.

The present invention can be performed in a step-wise fashion where after each step new reagents are added, or simultaneously, wherein all reagents are added at the initial step, or partially step-wise and partially simultaneously, wherein fresh reagent is added after a given number of steps. Additional materials may be added as necessary, for example, stabilizers. After the appropriate length of time has passed to produce the desired amount of the specific nucleic acid sequence, the reaction may be halted by inactivating the enzymes in any known manner or separating the components of the reaction.

Thus, in amplifying a nucleic acid molecule according to the present invention, the nucleic acid molecule is contacted with a composition preferably comprising a thermostable modified Pol III replicase in an appropriate amplification reaction mixture, preferably with stabilizers.

In one embodiment, the invention provides methods of amplifying large nucleic acid molecules, by a technique commonly referred to as “long range PCR” (Barnes, W. M., Proc. Natl. Acad. Sci. USA, 91:2216-2220 (1994) (“Barnes”); Cheng, S. et. al., Proc. Natl. Acad. Sci. USA, 91:5695-5699 (1994), which are hereby incorporated by reference). In one method, useful for amplifying nucleic acid molecules larger than about 5-6 kilobases, the composition with which the target nucleic acid molecule is contacted comprises not only a modified Pol III replicase, but also comprises a low concentration of a second DNA polymerase (preferably thermostable repair type polymerase, or a polC α subunit) that exhibits 3′-5′ exonuclease activity (“exo+” polymerases), at concentrations of about 0.0002-200 units per milliliter, preferably about 0.002-100 units/mL, more preferably about 0.002-20 units/mL, even more preferably about 0.002-2.0 units/mL, and most preferably at concentrations of about 0.40 units/mL. Preferred exo+polymerases for use in the present methods are Thermotoga maritima PolC, Pfu/DEEPVENT or Tli/NEN™ (Barnes; U.S. Pat. No. 5,436,149, which are hereby incorporated by reference); thermostable polymerases from Thermotoga species such as Tma Pol I (U.S. Pat. No. 5,512,462, which is hereby incorporated by reference); and certain thermostable polymerases and mutants thereof isolated from Thermotoga neapolitana such as Tne(3′exo+). The PolC product of Thermus thermophilus is also preferred. By using a modified Pol III replicase in combination with a second polymerase in the present methods, DNA sequences of at least 35-100 kilobases in length may be amplified to high concentrations with significantly improved fidelity.

For a discussion of long range PCR, see for example, Davies et al., Methods Mol. Biol. 2002; 187:51-5, expressly incorporated herein by reference.

Preferably, the amplification methods of the invention include the use of stabilizers with two-modified Pol III replicase. The stabilizers are preferably included in amplification reaction mixtures and increase the thermostability of the modified Pol III replicase in these reaction mixtures.

Amplification reaction mixtures of the present invention may include up to 25% co-solvent (total for all co-solvents added to a reaction mix), up to 5% crowding agent (total for all crowding agents added to a reaction mix) and up to 2M oxide (total for all oxides added to a reaction mix).

In an especially preferred embodiment, an amplification reaction mixture for use with a modified Pol II replicase derived from Aquifex aeolicus comprises TAPS-Tris (20 mM, pH8.7), 25 mM K₂SO₄, 10 mM NH₄(OAc)₂, 15 μmol ZnSO₄, and 4 mM MgSO₄.

In another especially preferred embodiment, an amplification reaction mixture for use with a modified Pol III replicase derived from Thermus thermophilus comprises HEPES-Bis-Tris-Propane (20 mM, pH7.5), 0.5 μmol ZnCl₂ or Zn(OAc)₂, and 6 mM Mg(OAc)₂.

In one embodiment, wherein one or more high temperature denaturation steps is done at less than 89° C., a thermocycling amplification method involves the use of a helicase in the thermocycling amplification reaction mixture, and preferably a helicase encoded by a bacterial dnaB gene. Helicases are preferably not used in thermocycling amplification methods involving one or more denaturation steps at or above 89° C.

In one embodiment, a nucleic acid replication method herein involves the use of a nucleic acid replication mixture that lacks ATP.

In one embodiment, a nucleic acid replication method herein involves the use of a nucleic acid replication mixture that lacks SSB, wherein SSB, if present in the replication reaction mixture, would inhibit the DNA polymerase activity of the particular minimal functional Pol III replicase used in the replication reaction mixture.

### Nucleic Acid Sequencing

In one aspect, the invention provides methods for sequencing a nucleic acid, preferably DNA, comprising subjecting the nucleic acid to a sequencing reaction in a sequencing reaction mixture comprising a modified Pol III replicase.

Preferably the modified Pol III replicases used lack 3′-5′ exonuclease activity capable of removing 3′ terminal dideoxynucleotides in the sequencing reaction mixture.

Accordingly, modified Pol III replicases comprising a polC encoded α subunit are generally not preferred for use in sequencing reactions, owing to their high level of zinc-independent 3′-5′ exonuclease activity.

In a preferred embodiment, the modified Pol III replicase comprises a dnaE α subunit, preferably of the genus Thermus or Aquifex, preferably of the species Thermus thermophilus, Thermus aquaticus, or Aquifex aeolicus.

Notably, the 3′-5′ exonuclease activity of dnaE α subunits used in the invention is generally capable of removing 3′ terminal dideoxynucleotides, while the 3′-5′ exonuclease activity of ε subunits is generally incapable of such terminal dideoxy nucleotide removal. Accordingly, modified Pol III replicases having 3′-5′ exonuclease activity which is conferred by an F subunit in a sequencing reaction mixture are generally useful in sequencing reactions herein. Moreover, undesirable dnaE α subunit 3′-5′ exonuclease activity is preferably reduced or completely inhibited through chemical means (i.e., buffer conditions, more particularly, Zn²⁺ concentration and pH).

Notably, DnaE from gram positive bacteria lacks 3′-5′ exonuclease activity capable of removing 3′ terminal dideoxynucleotides, making gram positive DnaE especially desirable for use in sequencing methods. Especially preferred is DnaE from Thermotoga maritima.

Nucleic acid molecules may be sequenced according to any of the literature-described manual or automated sequencing methods. Such methods include, but are not limited to, dideoxy sequencing methods (“Sanger sequencing”; Sanger, F., et al., J. Mol. Biol., 94:444-448 (1975); Sanger, F., et al., Proc. Natl. Acad. Sci. USA, 74:5463-5467 (1977); U.S. Pat. Nos. 4,962,022 and 5,498,523, which are hereby incorporated by reference), as well as by PCR based methods and more complex PCR-based nucleic acid fingerprinting techniques such as Random Amplified Polymorphic DNA (“RAPD”) analysis (Williams, J. G. K., et al., Nucl. Acids Res., 18(22):6531-6535, (1990), which is hereby incorporated by reference), Arbitrarily Primed PCR (“AP-PCR”) (Welsh, J., et al., Nucl. Acids Res., 18(24):7213-7218, (1990), which is hereby incorporated by reference), DNA Amplification Fingerprinting (“DAF”) (Caetano-Anolles et al., Bio/Technology, 9:553-557, (1991), which is hereby incorporated by reference), microsatellite PCR or Directed Amplification of Minisatellite-region DNA (“DAMD”) (Heath, D. D., et al., Nucl. Acids Res., 21(24): 5782-5785, (1993), which is hereby incorporated by reference), and Amplification Fragment Length Polymorphism (“AFLP”) analysis (Vos, P., et al., Nucl. Acids Res., 23(21):4407-4414 (1995); Lin, J. J., et al., FOCUS, 17(2):66-70, (1995), which are hereby incorporated by reference).

Once the nucleic acid molecule to be sequenced is contacted with the modified Pol III replicase in a sequencing reaction mixture, the sequencing reactions may proceed according to protocols disclosed above or others known in the art.

In an especially preferred embodiment, a sequencing reaction mixture for use with a modified Pol III replicase derived from Aquifex aeolicus comprises TAPS-Tris (20 mM, pH8.7), 25 mM K₂SO₄, 10 mM NH₄(OAc)₂, and 10 mM MgSO₄. Preferably, the reaction mixture lacks zinc so as to limit the 3′-5′ exonuclease activity of the α subunit.

In another especially preferred embodiment, a sequencing reaction mixture for use with a modified Pol III replicase derived from Thermus thermophilus comprises HEPES-Bis-Tris-Propane (20 mM, pH7.5), and 10 mM Mg(OAc)₂. Preferably, the reaction mixture lacks zinc so as to limit the 3′-5′ exonuclease activity of the α subunit.

In one aspect, the invention provides methods for simultaneous sequencing and amplification of DNA molecules in one homogenous reaction mixture, comprising subjecting the DNA molecules to a sequencing/amplification reaction in a sequencing/amplification reaction mixture comprising a modified Pol III replicase and a thermostable type I single subunit repair DNA polymerase.

In a preferred embodiment the sequencing/amplification reaction mixture used for a simultaneous sequencing/amplification reaction involving one or more high temperature denaturation steps comprises two RNA primers (forward and reverse) to drive the sequencing template amplification by the modified Pol III replicase, and a single DNA primer to drive the sequencing reaction by the repair type DNA polymerase. The repair type DNA polymerase preferably carries a mutated motif B sequence in which the conserved phenylalanine residue is replaced by a tyrosine residue. The modified Pol III replicase has an increased preference for RNA-primed template and preferably comprises one or more mutations in motif B. In one embodiment, the mixture further comprises stabilizers that contribute to the thermostability of the modified Pol III replicase.

In an alternative embodiment, a second modified Pol III replicase having increased ability to incorporate ddNTPs into primer extension products is used in place of the repair type DNA polymerase in a simultaneous sequencing/amplification reaction. The second modified Pol III replicase preferably comprises one or more mutations in motif B. In a preferred embodiment, the modified Pol III replicase additionally has increased preference for DNA-primed template.

In an alternative embodiment, the amplification and sequencing reactions are not simultaneous. In this embodiment, RNA-primers and DNA primers, and/or modified Pol III replicase and repair type DNA polymerase (or second modified Pol III replicase) are added sequentially to the same reaction mixture.

### Kits

In other preferred embodiments, the invention provides kits for use in nucleic acid amplification or sequencing, utilizing a two-component polymerase as disclosed herein.

A nucleic acid amplification kit according to the present invention comprises a two-component polymerase and dNTPs. The amplification kit encompassed by this aspect of the present invention may further comprise additional reagents and compounds necessary for carrying out standard nucleic acid amplification protocols (See U.S. Pat. Nos. 4,683,195 and 4,683,202, which are directed to methods of DNA amplification by PCR).

Similarly, a nucleic acid sequencing kit according to the present invention comprises a two-component polymerase and dideoxyribonucleoside triphosphates. The sequencing kit may further comprise additional reagents and compounds necessary for carrying out standard nucleic sequencing protocols, such as pyrophosphatase, agarose or polyacrylamide media for formulating sequencing gels, and other components necessary for detection of sequenced nucleic acids (See U.S. Pat. Nos. 4,962,020 and 5,498,523, which are directed to methods of DNA sequencing).

In a preferred embodiment, a kit includes buffers and stabilizers, or buffers with stabilizers.

In one embodiment, a kit lacks ATP and ATP is not used in the amplification reaction or the sequencing reaction provided for by the kit.

In additional preferred embodiments, the amplification and sequencing kits of the invention may further comprise a second DNA polymerase having 3′→5′ exonuclease activity. Preferred are

[symbol]

and mutants and derivatives thereof. Also preferred is the PolC product of Thermus thermophilus.

### Stabilizers

Preferably, a combination of at least two and more preferably at least three stabilizers is included in a thermocycling amplification reaction mixture. In preferred embodiments, the stabilizers include at least one co-solvent, such as a polyol (e.g. glycerol, sorbitol, mannitol, maltitol), at least one crowding agent, such as polyethylene glycol (PEG), ficoll, polyvinyl alcohol or polypropylene glycol, and a third component selected from the group consisting of sugars, organic quaternary amines, such as betaines, and their N-oxides and detergents. In particularly preferred embodiments, the stabilizers include a co-solvent, a crowding agent, and a quaternary amine N-oxide, such as trimethylamine-N-oxide (TMNO) or morpholino-N-oxide. In further preferred embodiments, the reaction mixture further comprises a fourth stabilizer, most preferably a second polyol. Other preferred four stabilizer combinations include three different co-solvents, and a quaternary amine N-oxide.

Nucleic acid replication reactions employing high temperature denaturation steps may benefit from the inclusion of one or more stabilizers in the reaction mixture. Preferred stabilizers in accordance with the present invention include co-solvents such as polyols and crowding agents such as polyethylene glycols, typically with one or more oxides, sugars, detergents, betaines and/or salts. Combinations of the foregoing components are most preferred.

As used herein, “crowding polymeric agent” or “crowding agent” refers to macromolecules that at least in part mimic protein aggregation. Illustrative crowding agents for use in the present invention include polyethylene glycol (PEG), PVP, Ficol, and propylene glycol.

As used herein, “detergent” refers to any substance that lowers the surface tension of water and includes, but is not limited to, anionic, cationic, nonionic, and zwitterionic detergents. Illustrative detergents for use in the present invention include Tween 20, NP-40 and Zwittergent 3-10.

As used herein, “polyol” refers to a polyhydric alcohols, i.e., alcohols containing three or more hydroxyl groups. Those having three hydroxyl groups (trihydric) are glycerols; those with more than three are called sugar alcohols, with general formula CH₂OH(CHOH)ᵣCH₂OH, where n may be from 2 to 5.

TABLE 1
Stabilizer Agents

| | Group II | Group III | | | | Group |
|---|---|---|---|---|---|---|
| Group I | (Co- | (Crowding | Group IV | Group V | Group VI | VII |
| (Sugars) | Solvents) | Agents) | (Detergents) | (Betaines) | (Salts) | (Oxides) |
| Trehalose | Glycerol | CM | Tween 20 | NDSB 195 | Potassium | TMNO |
| | | Cellulose | | | Glutamate | |
| Sucrose | Sorbitol | PEG 4000 | NP-40 | NDSB 201 | Sodium | |
| | | | | | Acetate | |
| β- | Mannitol | PEG 8000 | TritonX-100 | NDSB 256 | Sodium | |
| Cyclodextrin | | | | | Citrate | |
| α- | Maltitol | PEG | Pluronic Acid | 3-1-Pyridino- | | |
| Cyclodextrin | | 20000 | | 1-Propan- | | |
| | | | | Sulfonate | | |
| Glucose | 1-Methyl- | PVP | Zwittergent 3-10 | 4-Methyl- | | |
| | Pyrrolidinone | | | Morpholin-4- | | |
| | | | | Oxid | | |
| D-Fructose | 1- | Propylene | Zwittergent 3-12 | Betaine | | |
| | Methylindole | glycol | | Monohydrate | | |
| D-Mannose | 2- | | Zwittergent 3-14 | Betaine | | |
| | Pyrrolidinone | | | Hydrochloride | | |
| D- | Acetamide | | Zwittergent 3-16 | New Betaine | | |
| Galactose | | | | | | |
| | | | Chaps | | | |
| | | | ChapsSO | | | |
| | | | N-Octyl-Sucrose | | | |
| | | | Caprolyl | | | |
| | | | Sulfobetaine SB 3- | | | |
| | | | 10 | | | |
| | | | Myristyl- | | | |
| | | | Sulfobetaine SB 3- | | | |
| | | | 14 | | | |
| | | | N-Octyl-β- | | | |
| | | | glucopyranosid | | | |
| | | | N-Octyl-β-D- | | | |
| | | | thioglucopyranosid | | | |

TABLE 2
Preferred Stabilizer Combinations

| Preferred | Preferred | Preferred | Preferred | Preferred |
|---|---|---|---|---|
| Embodiment | Embodiment | Embodiment | Embodiment | Embodiment |
| 1 | 2 | 3 | 4 | 5 |
| Glycerol | Glycerol | Glycerol | Glycerol | Glycerol |
| Sorbitol | Maltitol | Maltitol | Maltitol | Maltitol |
| PEG (20K) | PEG (20K) | Sorbitol | Sorbitol | PEG (20K) |
| TMNO | TMNO | TMNO | PEG (20K) | Betaine |

Embodiments of the present invention generally include combining at least two and more preferably at least three different stabilizers selected from Groups I-VII (see Table 2) together to facilitate temperature based nucleic acid amplification. Preferred embodiments of the present invention include a combination of at least one member from Group II with a member from Group III within the amplification reaction mixture, particularly where the member from Group II is glycerol and/or sorbitol. Particularly preferred combinations include two different members of Group II combined with one member from Group III and one member from Group VI.

### Diagnostic Methods

In one aspect, the invention provides compositions and methods for detecting the presence of bacteria. The methods involve analyzing a sample from the host for the presence of a bacterial DNA Pol III enzyme. As replicases are critical to the viability of bacteria, bacterial DNA Pol III enzymes are extremely useful diagnostic markers that are indicative of the presence of viable bacteria.

In one embodiment, compositions and methods for detecting the presence of viable bacteria in a host are provided. In a preferred embodiment, the methods involve analyzing a sample from the host for the presence of an RNA transcript encoding a bacterial Pol III enzyme.

A host sample may be, for example, a fluid sample from a host suspected of having a bacterial infection.

In some embodiments, the methods involve the use of PCR to detect a bacterial DNA Pol III enzyme. In one embodiment, the method involves use of a first PCR primer that hybridizes to a nucleotide sequence encoding a bacterial DNA Pol III motif C, and a second PCR primer that hybridizes to the complement of a nucleotide sequence encoding a bacterial DNA Pol III motif B. PCR is done using the two primers and PCR products are probed with an oligonucleotide probe that hybridizes to a nucleotide sequence encoding a bacterial DNA Pol III motif A, or its complement. In one embodiment, PCR products are combined with a microarray comprising such an oligonucleotide probe that hybridizes to a nucleotide sequence encoding a bacterial DNA Pol III motif A, or its complement. In one embodiment, the methods further comprise determining the spacing of bacterial DNA Pol III motifs C, A, and B from the PCR product. The formation of a PCR product with such primers, wherein the product is determined to comprise an internal bacterial DNA Pol III motif A, evidences the presence of a bacterial DNA Pol III enzyme, and the presence of bacteria in the host. In one embodiment, the methods further comprise determining the spacing of bacterial DNA Pol III motifs C, A, and B from the PCR product.

### Drug Screening

In one aspect, the invention provides compositions and methods for screening candidate bioactive agents for the ability to modulate, preferably inhibit, the activity of bacterial DNA Pol III enzymes. Candidate bioactive agents obtained by the screening methods described herein find use in the treatment of patients having a bacterial infection.

In a preferred embodiment, the methods involve screening for binding of a candidate bioactive agent to a bacterial DNA Pol III enzyme identified by the classification methods described herein.

In another preferred embodiment, the methods involve screening for binding of a candidate bioactive agent to one or more of bacterial DNA Pol III motifs C, A, and B, derived from a bacterial DNA Pol III enzyme. In a preferred embodiment, the methods involve use of a fragment of a bacterial DNA Pol III enzyme comprising one or more of bacterial DNA Pol III motifs C, A, and B in a binding assay with a candidate bioactive agent. In a preferred embodiment, the methods further comprise screening a candidate bioactive agent for an inability to bind to one or more of human replicase motifs A, B, and C. Preferably, a fragment of a human replicase comprising one or more of human replicase motifs A, B, and C is used.

The term “candidate bioactive agent” or “candidate agent” as used herein describes any molecule, e.g., protein, small organic molecule, carbohydrate (including polysaccharide), polynucleotide, lipid, etc. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are organic chemical moieties or small molecule chemical compositions, a wide variety of which are available in the literature.

Provisional application Ser. No. 60/560,793, titled “DNA Polymerase III α Subunit”, and filed 7 Apr. 2004, is expressly incorporated herein in its entirety by reference.

Citations herein are expressly incorporated herein in their entirety by reference.

### EXAMPLES

### Example 1

### Primer Extension by T.th α Subunit and Tth DNA Pol III Holoenzyme

(Figure X) Thermus thermophilus (“T.th”) α subunit was used in a time course primer extension assay to compare its extension rate as a stand alone polymerase to that of the minimal T.th DNA Pol III holoenzyme. In 19.6 μl reaction mixes 350 ng (0.15 pmol) of ssM13mp18 DNA primed with 0.375 pmol of a 30 mer oligodeoxynucleotide primer were incubated at 60° C. for 2 minutes in the presence of 2 μg (15 pmol) of T.th α subunit in 20 mM TAPS-Tris (pH 7.5), 8 mM Mg(OAc)₂, 14% glycerol, 40 μg/ml BSA and 40 mM Sorbitol. The primer extension/replication was started by adding 0.4 μl of a dNTP mix containing 10 mM dATP, 10 mM dGTP, 10 mM dTTP, and 10 mM dCTP to the final concentration of 200 μmol each. The indicated time points of the primer extension assay were taken stopping individual reactions by addition of 2 μl 0.1M EDTA and transferring them on ice. The replication products were analyzed by electrophoretic separation in a 0.7% TEAE-buffered agarose gel with subsequent ethidium bromide staining. The arrow marks the first time point at which the full-size (7.2 kb) double-stranded replication product was detectable. The α-subunit alone is capable of replicating a DNA-primed 7.2 kb M13 template with a maximum extension rate of 240 b/sec. That is about 6-8× faster then the extension rate of Taq DNA polymerase I (30-40-b/sec) under equivalent conditions. The extension rate of the minimal holoenzyme with clamp loader and processivity clamp is about 3× faster (725 b/sec) than the replication speed of a alone.

### Example 2

### Primer Extension by T.ma α Subunit (PolC)

(Figure X) Thermotoga maritima (“T.ma”) α subunit was used in a time course primer extension assay to examine its extension rate as a stand alone polymerase. In 19.6 μl reaction mixes” 350 ng (0.15 pmol) of ssM13mp18 DNA primed with 0.375 pmol of a 30-mer oligodeoxynucleotide primer were incubated at 60° C. for 2 minutes in the presence of 100 ng (0.64 pmol) of Tma DNA Pol III alpha subunit (polC) in 20 mM TAPS-Tris (pH 7.5), 25 mM KCl, 10 mM (NH₄)₂SO₄, 8 mM Mg(OAc)₂, 14% glycerol, 40 mg/ml BSA and 40 mM Sorbitol. The primer extension/replication was started by adding 0.4 μl of a dNTP mix containing 10 mM dATP, 10 mM dGTP, 10 mM dTTP, and 10 mM dCTP to the final concentration of 200 μmol each. The indicated time points of the primer extension assay were taken stopping individual reactions by addition of 2 μl 0.1M EDTA and transferring them on ice. The replication products were analyzed by electrophoretic separation in a 0.7% TEAE-buffered agarose gel with subsequent ethidium bromide staining. The arrow marks the first time point at which the full-size (7.2 kb) double-stranded replication product was detectable. The T.ma α subunit (polC) replicated the 7.2 kb M13 template with an extension rate of 720 b/sec.

### Example 3

### Deoxyribonucleotide/Ribonucleotide Primer Discrimination

Thermus thermophilius mutants with amino acid substitutions in one or multiple motifs within the α subunit are compared to a non-mutated Thermus thermophilus in a time course primer extension assay to determine their ability to discriminate between DNA primers and RNA primers. In 19.6 μl reaction mixes 350 ng (0.15 pmol) of ssM13mp18 DNA primed with 0.375 pmol of a 30-mer primer (either DNA or RNA) are incubated at 60° C. for 2 minutes in the presence of 100 ng (0.64 pmol) of either mutated or non-mutated Tth DNA Pol III alpha subunit in 20 mM TAPS-Tris (pH 7.5), 25 mM KCl, 10 mM (NH₄)₂SO₄, 8 mM Mg(OAc)₂, 14% glycerol, 40 mg/ml BSA and 40 mM Sorbitol. The primer extension/replication is started by adding 0.4 μl of a dNTP mix containing 10 mM dATP, 10 mM dGTP, 10 mM dTTP, and 10 mM dCTP to the final concentration of 200 μmol each. The indicated time points of the primer extension assay are taken stopping individual reactions by addition of 2 μl 0.1M EDTA and transferring them on ice. The replication products are analyzed by electrophoretic separation in a 0.7% TEAE-buffered agarose gel with subsequent ethidium bromide staining. An extension time ratio is generated by dividing the extension rate of the non-mutated Tth DNA Pol III alpha subunit by the extension rate of the mutated Tth DNA Pol III alpha subunit for each primer type. Ratios equal to 1 indicate that the mutated Tth and non-mutated Tth can utilize a specific primer type with equal efficiency. Ratios of greater than 1 indicate that the Tth mutant utilizes a specific type with less efficiency than the non-mutated Tth. Ratios less than 1 indicate that the Tth mutant utilizes a specific primer type with greater efficiency than the non-mutated Tth

### Example 4

### Dideoxyribonucleotide Incorporation

The following assay is used to assess various DnaE and PolC mutants for their ability to incorporate dideoxyribonucleotides into a pre-primed nucleotide substrate. The following partially double stranded substrate is provided for the assay:

5′ - XXXACG
3′ - XXXTGCGTACTCCTATCATCT

The pre-primed nucleotide substrate is added to a reaction mixture comprising a buffer (as indicted above), DnaE or PolC, deoxyribonucleotides, and FAM labeled dideoxyribonucleotide (ddCTP). The mixture is incubated for 5 minutes at 60-70° C. After the reaction is complete, it can be quenched by the addition of EDTA. The reaction mixture is purified to remove any residual labeled and unlabeled nucleotides. The reaction mixture is then placed into a microtitre plate and any incorporated fluorescence is read via a standard spectrophotometer. A non-labeled blank or standard is used for reference to compare the fluorescent reading collected under a 500-540 nm setting. Any DnaE mutant or PolC mutant that can incorporate ddNTPS will generate a higher fluorescent reading than that of the standard or blank.

### Example 5

### Labeled (Bulky) Nucleotide Incorporation/Extension

The following assay is used to assess various DnaE and PolC mutants for their ability to incorporate dideoxyribonucleotides into a pre-primed nucleotide substrate. The following partially double stranded substrate is provided for the assay:

5′ - XXXACG
3′ - XXXTGCGTACTCCTATCATCT

The pre-primed nucleotide substrate is added to a reaction mixture comprising a buffer (as indicted above), DnaE or PolC, dNTPs, FAM labeled dCTP, and P³² labeled dTTP. The mixture is incubated for 5 minutes at 60-70° C. After the reaction is complete, it can be quenched by the addition of EDTA. The reaction mixture is purified to remove any residual labeled and unlabeled nucleotides. The reaction mixture is then placed into a microtitre plate and any incorporated fluorescence is read via a standard spectrophotometer. A non-labeled blank or standard is used for reference to compare the fluorescent reading collected under a 500-540 nm setting. Any DnaE mutant or PolC mutant that can incorporate labeled dNTPS will generate a higher fluorescent reading than that of the standard or blank. Once a spectrophotometric reading is taken, the sample is then placed into a scintillation counter to determine the level of P³² incorporation, A non-FAM labeled blank or standard is used for comparison. Samples that can extend the substrate after the FAM labeled dCTP will have a higher level of P³² incorporation than that of the blank or standard. The higher level of P³² incorporation will result in a higher CPM reading on the scintillation counter and indicate a mutant that is capable of template extension after labeled (bulky) nucleotide incorporation.

### Example 6

### Simultaneous Amplification and Sequencing

Based on the ability of any DnaE alpha subunit to utilize RNA primers for DNA synthesis and to discriminate against the incorporation of ddNTP's versus dNTPs and based on the ability of the AmpliTaq FS Sequencing DNA polymerase or T7 DNA Sequenase to incorporate ddNTPs efficiently, but to discriminate against the extension of RNA primers, template sequencing and template amplification can be run simultaneously in one homogenous reaction.

This experiment provides a 2.9 kb double stranded linear DNA substrate. This DNA substrate is added to a reaction mixture comprising a buffer, dNTPs, labeled ddNTPs, forward and reverse RNA primers for template amplification and one DNA primer to drive the sequencing reaction and two different DNA polymerase: a wild-type DnaE alpha subunit of DNA Pol III and a mutated AmpliTaq FS sequencing polymerase. This reaction mixture is cycled through the following incubation temperatures for at least 30 times: 93° C. for 15 seconds, 55° C. 2 minutes. The DNA sequencing primer is designed as such that it anneals between the annealing sites of the RNA primers for template amplification. The DnaE alpha subunit driving the template amplification reaction can utilize RNA primers, but cannot incorporate deoxyribonucleotides and the AmpliTaq FS sequencing polymerase can incorporate ddNTPS but extends only the DNA sequencing primer. In the specific case, the DnaE alpha subunit can amplify a pGEM substrate using RNA primers. The following RNA amplification primers are provided:

RNA Forward Primer
(5′ - GACGUUGUAAAACGACGGCCAGU - 3′)
RNA Reverse Primer
(5′ - GUGACUGGGAAAACCCUGGCGUUAC - 3′)

The AmpliTaq FS sequencing polymerase lacks the ability to amplify the substrate using the RNA primers but can utilize the DNA primer for extension while incorporating ddNTPs. In this specific case, the AmpliTaq FS sequencing polymerase is used as the sequencing enzyme because it has the ability to incorporate dideoxyribonucleotide chain terminators used in standard Sanger Sequencing protocols. The AmpliTaq FS sequencing polymerase utilizes a single DNA sequencing primer that is internal to the RNA forward amplification primer. The following DNA sequencing primer is provided:

DNA Sequencing Primer
(5′ - CACAATTCCACACAACATACGAGCCGG - 3′)

The reaction mixture is temperature cycled from 55-95° C. for a plurality of cycles. During the cycling process, the DnaE alpha subunit utilizes the RNA primers to amplify a pGEM substrate while the AmpliTaq FS sequencing polymerase simultaneously generates labeled chain terminated copies of a portion of the substrate by way of the single DNA sequencing primer. After the temperature cycling is complete, the reaction mixture can be purified to remove any residual labeled or unlabeled nucleotides, as well as residual salts, and analyzed by a variety of sequencing methods (i.e. capillary electrophoresis).

### Example 7

### Alpha Subunit Mutant Generation

The site-specific mutagenesis of a gene encoding a DNA Pol III alpha subunits can be carried out by any method of site-specific mutagenesis known in the prior art using commercially available kits according to the manufacturer's instructions.

For example, a linear, double-stranded plasmid template carrying the dnaE gene coding for the desired DNA Pol III alpha subunit for mutagenesis is created by inverse PCR. The forward and reverse primers for the inverse PCR are designed to anneal head-to-head (5′-end to 5′-end) at the mutagenic site in the dnaE coding sequence. A complete linear, double-stranded copy of the plasmid is than amplified in 35 cycles of the following PCR program: 20 seconds 93° C., 5 minutes 65° C. The resulting amplification product has a blunt, double-strand break at the site targeted for mutagenesis. A phosphorylated, double-stranded mutagenic codon cassette is then inserted at the target site by ligation with T4 DNA ligase. The mutagenic cassette is formed by hybridization of two complementary deoxyoligonucleotides phosphorylated at their 5′-termini.

Each mutagenic codon cassette contains a three base pair direct terminal repeat and two head-to-head recognition sequences for the restriction endonuclease Sap I, an enzyme that cleaves outside of its recognition sequence. The sequence of the three base pair repeat resembles the desired mutated codon. The intermediate molecule containing the mutagenic cassette is then digested with Sap I, thereby removing most of the mutagenic cassette, leaving only a three base cohesive overhang that is ligated to generate the final insertion or substitution mutation. Because the mutagenic cassette is excised during this procedure and alters the target only by introducing the desired mutation, the same cassette can be used to introduce a particular codon at all target sites. The approach allows for the generation of any desired mutation of any DNA Pol III alpha subunit at any position. If several mutations are desired in the same DNA Pol III alpha subunit, the described process shall be repeated sequentially using several mutagenic cassettes amplifying the intermediate mutated plasmid molecules by inverse PCR. The resulting mutated molecule can then be transformed in the desired host, expressed, purified, and assayed for desired effect.

1. A Pol III α mutant having at least one mutation in one or more of motifs A and B, wherein said modified Pol III α has altered activity relative to an unmodified Pol III α not having said at least one mutation.

2. The Pol III α mutant according to claim 1, wherein said Pol III α mutant has altered dNTP discrimination activity relative to said unmodified Pol III α.

3. The Pol III α mutant according to claim 2, wherein said Pol III α mutant has increased affinity for a ddNTP.

4. The Pol III α mutant according to claim 2, wherein said Pol III α mutant comprises one or more mutations in motif B.

5. The Pol III α mutant according to claim 2, wherein said Pol III α mutant has increased affinity for a labeled nucleotide.

6. The Pol III α mutant according to claim 5, wherein said Pol III α mutant comprises one or more mutations in motif A.

7. The Pol III α mutant according to claim 5, wherein said Pol III α mutant comprises one or more mutations in motif B.

8. The Pol III α mutant according to claim 5, wherein said Pol III α mutant comprises one or more mutations in motif A and one or more mutations in motif B.

9. The Pol III α mutant according to claim 1, wherein said Pol III α mutant has altered primer discrimination activity relative to said unmodified Pol III α.

10. The Pol III α mutant according to claim 9, wherein said Pol III α mutant has increased affinity for RNA-primed template.

11. The Pol III α mutant according to claim 9, wherein said Pol III α mutant has decreased affinity for DNA-primed template.

12. The Pol III α mutant according to claim 9, wherein said Pol III α mutant has increased affinity for DNA-primed template.

13. The Pol III α mutant according to claim 9, wherein said Pol III α mutant has decreased affinity for RNA-primed template.

14. The Pol III α mutant according to claim 9, wherein said Pol III α mutant comprises one or more mutations in motif B.

15. A modified Pol III replicase, comprising a Pol III α mutant according to any one of claims 1-14.

16. The modified Pol III replicase according to claim 15, wherein said modified Pol III replicase lacks a clamp loader.

17. The modified Pol III replicase according to claim 15, wherein said modified Pol III replicase comprises a β sliding clamp.

18. A method for classifying a candidate polypeptide as a bacterial DNA Pol III α, comprising identifying in said candidate polypeptide at least one of bacterial DNA Pol III α motifs A, B or C.

19. The method according to claim 18, comprising identifying in said candidate polypeptide said bacterial DNA Pol III α motifs A and B.

20. The method according to claim 19, further comprising determining the arrangement of said bacterial DNA Pol III α motifs A and B.

21. The method according to claim 20, further comprising determining the spacing of said bacterial Pol III α motifs A and B.

22. The method according to claim 19, further comprising identifying in said candidate polypeptide bacterial DNA Pol III α motif C.

23. The method according to claim 22, further comprising determining the arrangement of said bacterial DNA Pol III α motifs C and A, C and B, or C and A and B.

24. The method according to claim 23, further comprising determining the spacing of said bacterial Pol III α motifs C and A, C and B, or C and A and B.

25. The method according to claim 18, wherein said bacterial DNA Pol III α motifs correspond to gram positive bacteria.

26. The method according to claim 18, wherein said candidate polypeptide is derived from a human sample.

27. A nucleic acid amplification kit, comprising a Pol III α mutant according to claim 1.

28. A nucleic acid amplification reaction tube, comprising a Pol III α mutant according to claim 1.

29. A nucleic acid amplification reaction mixture, comprising a Pol III α mutant according to claim 1.

30. A method of replication nucleic acid, comprising subjecting said nucleic acid to a replication reaction in a replication reaction mixture comprising a Pol III α mutant according to claim 1.

31. A method for amplifying and sequencing nucleic acid in a single reaction mixture, comprising subjecting said nucleic acid to a simultaneous amplification and sequencing reaction in a reaction mixture comprising a Pol III α mutant according to claim 1.

32. A method for diagnosing a patient as having a bacterial infection, comprising identifying in a candidate polypeptide obtained from said patient bacterial DNA Pol III α motifs A, B, or C, or a combination thereof.

33. The method according to claim 32, comprising identifying in said candidate polypeptide at least two of said bacterial DNA Pol III α motifs.

34. The method according to claim 33, further comprising determining the arrangement of said at least two bacterial DNA Pol III α motifs in said candidate polypeptide.

35. The method according to claim 34, further comprising determining the spacing of said at least two bacterial DNA Pol III α motifs in said candidate polypeptide.

36. The method according to claim 35, wherein said bacterial DNA Pol III α motifs are gram positive bacterial Pol III consensus motifs.

37. A method for diagnosing a patient as having a bacterial infection, comprising obtaining a sample from said patient, and identifying in said sample a nucleic acid comprising one or more nucleotide sequences encoding one or more of bacterial DNA Pol III α motifs A, B, and C.

38. The method according to claim 37, wherein said nucleic acid comprises at least two of said nucleotide sequences encoding bacterial DNA Pol III α motifs A, B, and C.


SEQUENCE LISTING (93 sequences)

SEQ ID NO: 1 - protein, 5 aa, Escherichia coli
QADMF

SEQ ID NO: 2 - protein, 5 aa, Escherichia coli
  misc_feature (3)..(3): Xaa may be Serine or Aspartic Acid
QLXLF

SEQ ID NO: 3 - protein, 6 aa, Escherichia coli
QVELEF

SEQ ID NO: 4 - protein, 5 aa, Escherichia coli
QLDLF

SEQ ID NO: 5 - protein, 7 aa, Artificial
  Synthetic
GMMGLFS

SEQ ID NO: 6 - protein, 6 aa, Artificial
  Synthetic
QEAVPF

SEQ ID NO: 7 - protein, 7 aa, Artificial
  Synthetic
GLVGLFA

SEQ ID NO: 8 - protein, 6 aa, Artificial
  Synthetic
EEVVPF

SEQ ID NO: 9 - protein, 7 aa, Artificial
  Synthetic
GALDAFG

SEQ ID NO: 10 - protein, 7 aa, Artificial
  Synthetic
TQNSLFG

SEQ ID NO: 11 - protein, 6 aa, Artificial
  Synthetic
GVKVII

SEQ ID NO: 12 - protein, 6 aa, Artificial
  Synthetic
GAFDFT

SEQ ID NO: 13 - protein, 7 aa, Artificial
  Synthetic
XLLXLFX

SEQ ID NO: 14 - protein, 7 aa, Artificial
  Synthetic
XQLXLFX

SEQ ID NO: 15 - protein, 7 aa, Artificial
  Synthetic
XNLXLFX

SEQ ID NO: 16 - protein, 6 aa, Artificial
  Synthetic
QLXLXF

SEQ ID NO: 17 - protein, 7 aa, Artificial
  Synthetic
GXXXLFG

SEQ ID NO: 18 - protein, 21 aa, Thermosynechococcus elongatus
PDTEKLRQEKDLLGFYVSNHP

SEQ ID NO: 19 - protein, 21 aa, Thermus aquaticus
AETRERGRSGLVGLFAEVEEP

SEQ ID NO: 20 - protein, 20 aa, Thermus aquaticus
SLVKAGALDAFGDRARLLAS

SEQ ID NO: 21 - protein, 21 aa, Thermus thermophilus
AETREKARSGMMGLFSEVEEP

SEQ ID NO: 22 - protein, 22 aa, Aquifex aeolicus
ANSEKALMATQNSLFGAPKEEV

SEQ ID NO: 23 - protein, 24 aa, Deinococcus radiodurans
AEINARAQSGMSMMFGMEEVKKER

SEQ ID NO: 24 - protein, 20 aa, Deinococcus radiodurans
SLIKSGAFDAFGERHQLIES

SEQ ID NO: 25 - protein, 23 aa, Escherichia coli
ADQHAKAEAIGQADMFGVLAEEP

SEQ ID NO: 26 - protein, 22 aa, Wolinella sp.
SKESERAKRMAENSLFGDSEEM

SEQ ID NO: 27 - protein, 23 aa, Wolbachia sp.
NKNKQDKESSQAALFGSLDVLKP

SEQ ID NO: 28 - protein, 21 aa, Tropheryma sp.
GEIQIMLSGQNYNNFSEQLVT

SEQ ID NO: 29 - protein, 21 aa, Treponema sp.
GVRQHQTKKGAMMGFGTFEDL

SEQ ID NO: 30 - protein, 21 aa, Shigella sp.
QHAKAEAIGQADMFGVLAEEP

SEQ ID NO: 31 - protein, 21 aa, Shewanella sp.
QHAKAEAIGQHDMFGLLNSDP

SEQ ID NO: 32 - protein, 21 aa, Salmonella sp.
QHAKAEAIGQTDMFGVLAEEP

SEQ ID NO: 33 - protein, 21 aa, Rickettsia sp.
FALGAIKGVTPNFGKLVTDER

SEQ ID NO: 34 - protein, 20 aa, Rickettsia sp.
TAYHEEQESNQFSLIKVSSL

SEQ ID NO: 35 - protein, 18 aa, Rickettsia sp.
TLAFYEFEAMGLFISNHP

SEQ ID NO: 36 - protein, 21 aa, Rickettsia sp.
TLVLSDPENIFELSIFSEEVL

SEQ ID NO: 37 - protein, 20 aa, Rhodopseudomonas sp.
NHEAATSGQNDMFGGLSDAP

SEQ ID NO: 38 - protein, 21 aa, Pseudomonas aeruginosa
QTARSHDSGHMDLFGGVFAEP

SEQ ID NO: 39 - protein, 21 aa, Porphyromonas sp.
TMESLALAGAFDSFALSREEY

SEQ ID NO: 40 - protein, 21 aa, Pirellula sp.
QADKKTGQASFFDAFDEEVDA

SEQ ID NO: 41 - protein, 21 aa, Pasteurella sp.
QHAKDAAMGQADMFGVLTESH

SEQ ID NO: 42 - protein, 21 aa, Neisseria sp.
QKAANANQGGLFDMMEDAIEP

SEQ ID NO: 43 - protein, 21 aa, Mycobacterium leprae
GTKKAEAIGQFDLFGGTDGGT

SEQ ID NO: 44 - protein, 21 aa, Mycobacterium bovis
QVEALATAGALGCFGMSRREA

SEQ ID NO: 45 - protein, 21 aa, Leptospira sp.
QERANEGQFSLFGNEESSFSL

SEQ ID NO: 46 - protein, 22 aa, Helicobacter sp.
AKDKANEMMQGGNSLFGAMEGG

SEQ ID NO: 47 - protein, 21 aa, Haemophilus sp.
QHSKMEALGQSDMFGVLTETP

SEQ ID NO: 48 - protein, 21 aa, Geobacter sp.
QEKESAQVSLFGAEEIVRTNG

SEQ ID NO: 49 - protein, 21 aa, Erwinia sp.
QHAKAEAIGQVDMFGVLADAP

SEQ ID NO: 50 - protein, 20 aa, Desulfovibrio sp.
LKELMTKKGQRMAFAGVEDL

SEQ ID NO: 51 - protein, 20 aa, Coxiella sp.
QRNRDMILGQHDLFGEEVKG

SEQ ID NO: 52 - protein, 22 aa, Coxiella sp.
EKETLGLYVSGHPLQACIKEMK

SEQ ID NO: 53 - protein, 19 aa, Corynebacterium sp.
TESLIKAGAFDSMDHPRKG

SEQ ID NO: 54 - protein, 25 aa, Corynebacterium sp.
TSTKKAADKGQFDLFAGLGADAEEV

SEQ ID NO: 55 - protein, 21 aa, Thermotoga maritima
VALEMILRGFSFLPPDIFKSD

SEQ ID NO: 56 - protein, 21 aa, Streptococcus pyogenes
KLDLYKSDAIEFQIKGDTLIP

SEQ ID NO: 57 - protein, 21 aa, Staphylococcus aureus
PISLEKSQAFEFIIEGDTLIP

SEQ ID NO: 58 - protein, 21 aa, Staphylococcus epidermidis
PISLEKSQAFDFIIEGDTLIP

SEQ ID NO: 59 - protein, 21 aa, Streptococcus thermophilus
QLDLYKSQATEFLIEGDTLIP

SEQ ID NO: 60 - protein, 21 aa, Streptococcus pneumoniae
KLDLYRSQATEFLIDGDTLIP

SEQ ID NO: 61 - protein, 22 aa, Enterococcus faecalis
MIDLYKSDAENFVIEGDTLIAP

SEQ ID NO: 62 - protein, 21 aa, Bacillus haldourans
KVDLYRSEATEFLVEGNTLIP

SEQ ID NO: 63 - protein, 21 aa, Bacillus licheniformis
KVDLYRSSASEFIIDGNSLIP

SEQ ID NO: 64 - protein, 21 aa, Lactococcus lactis
KIDLYRSEATEFVIDGDTLIP

SEQ ID NO: 65 - protein, 21 aa, Clostridium acetobutylicum
PIDIYKSHATKFLVEEDGLRP

SEQ ID NO: 66 - protein, 21 aa, Oceanobacillus iheyensis
KVDLYQSSATDFIVEGDSLLP

SEQ ID NO: 67 - protein, 21 aa, Thermoanaerobacter sp.
NVDLYRSDAEKFLITEEGLLP

SEQ ID NO: 68 - protein, 56 aa, Artificial
  Taq
KRALESLVKAGALDAFGDRARLLASLEPLLRWAAETRERGRSGLVGLFAEVEEPPL

SEQ ID NO: 69 - protein, 56 aa, Thermus thermophilus
KRTLESLIKAGALDGFGERARLLASLEGLLRWAAETREKARSGMMGLFSEVEEPPL

SEQ ID NO: 70 - protein, 52 aa, Aquifex sp.
KKVVEALVKAGAFDFTKKKRKELLAKVANSEKALMATQNSLFGAPKEEVEEL

SEQ ID NO: 71 - protein, 61 aa, Deinococcus radiodurans
RKALESLIKSGAFDAFGERHQLIESLEDALEDAAGTAEINARAQSGMSMMFGMEEVKKERP

SEQ ID NO: 72 - protein, 59 aa, Coxiella sp.
RNRDMILGQHDLFGEEVKGIDEDYTEVPEWNDSDRLRGEKETLGLYVSGHPLQACIKEM

SEQ ID NO: 73 - protein, 59 aa, Corynebacterium sp.
KRVTESLIKAGAFDSMDHPRKGLLLIHEDAVDAVTSTKKAADKGQFDLFAGLGADAEEV

SEQ ID NO: 74 - protein, 60 aa, Chromobacterium sp.
KRVIEALIRAGAFDAIEPNRALLFANVGLAMEAAEQAHANANQGGLFDMFGDDVAPAVEM

SEQ ID NO: 75 - protein, 59 aa, Chlamydophilia sp.
KKHTENLIDAGCFDVFESDRDVAQATLETLYDTISKEKKEAATGVMTFFSLNTMHQEHR

SEQ ID NO: 76 - protein, 59 aa, Chlamydia sp.
KKQLESLVDAGSFNCFEPNKDLAIAILNDLYDTFSREKKEAATGVLTFFSLNSMTKDPV

SEQ ID NO: 77 - protein, 58 aa, Candidatus sp.
KLSIDSIVASRDCDGEFHSLFDFCVRVDNSKVNQRIIEKLIYSGAFDFFGIHRSELIS

SEQ ID NO: 78 - protein, 59 aa, Borellia sp.
KKFLESAIKSGLFDSLDQNRKTLFENLDHLIEVVSEDKNNKKLGQNSLFGALESQDPIQ

SEQ ID NO: 79 - protein, 60 aa, Bordetella sp.
RRTIEALIKAGAFDTIEPNRAAMLASVGTAMEAAEQAARSANQSSLFGDDSGDVVAGELT

SEQ ID NO: 80 - protein, 62 aa, Bdellovibrio sp.
KKVIECLIKAGAFDGFGAHRAQLVAGYQKYLDRAIGLQKDREMGQSSLFDLGPSTETKVTLE

SEQ ID NO: 81 - protein, 58 aa, Acinetobacter sp.
KRTLEALIRAGALDCLQIERSSLMAQLPEAVQAAEQARQNRETGIMDLFGEVEEVQRK

SEQ ID NO: 82 - protein, 59 aa, Xyellana sp.
RRALEAMIHAGALDELGKNRASVMLQLPEVIKATEQMSRERESGQNSLFGNADPGTPVI

SEQ ID NO: 83 - protein, 59 aa, Wolinella sp.
KKALESLIKSGSMVGFGYSRRALLEQIEAINEASKESERAKRMAENSLFGDSEEMIVAR

SEQ ID NO: 84 - protein, 59 aa, Wolbachia sp.
KRALESLIKSGAFDSVHKNRKQLYESMDTLIYFANKNKQDKESSQAALFGSLDVLKPKL

SEQ ID NO: 85 - protein, 59 aa, Shigella sp.
RRVLEKLIMSGAFDRLGPHRAALMNSLGDALKAADQHAKAEAIGQADMFGVLAEEPEQI

SEQ ID NO: 86 - protein, 59 aa, Shewanella sp.
KRVIEKLICAGALDALGPHRASMMATLPEAISAADQHAKAEAIGQHDMFGLLNSDPEDS

SEQ ID NO: 87 - protein, 59 aa, Salmonella sp.
RRVLEKLIMSGAFDRLGPHRAALMNSLGDALKAADQHAKAEAIGQTDMFGVLAEEPEQI

SEQ ID NO: 88 - protein, 59 aa, Vibrio vulnificus
KRVIEKLIYAGALDRLGPHRAALMASLDDAVKAASQHHQAEAFGQADMFGVLTDAPEEV

SEQ ID NO: 89 - protein, 1160 aa, Escherichia coli
MSEPRFVHLRVHSDYSMIDGLAKTAPLVKKAAALGMPALAITDFTNLCGLVKFYGAGHGAGIKPIVGADFNVQCDLLGDELTHLTVLAANNTGYQNLTLLISKAYQRGYGAAGPIIDRDWLIELNEGLILLSGGRMGDVGRSLLRGNSALVDECVAFYEEHFPDRYFLELIRTGRPDEESYLHAAVELAEARGLPVVATNDVRFIDSSDFDAHEIRVAIHDGFTLDDPKRPRNYSPQQYMRSEEEMCELFADIPEALANTVEIAKRCNVTVRLGEYFLPQFPTGDMSTEDYLVKRAKEGLEERLAFLFPDEEERLKRRPEYDERLETELQVINQMGFPGYFLIVMEFIQWSKDNGVPVGPGRGSGAGSLVAYALKITDLDPLEFDLLFERFLNPERVSMPDFDVDFCMEKRDQVIEHVADMYGRDAVSQIITFGTMAAKAVIRDVGRVLGHPYGFVDRISKLIPPDPGMTLAKAFEAEPQLPEIYEADEEVKALIDMARKLEGVTRNAGKHAGGVVIAPTKITDFAPLYCDEEGKHPVTQFDKSDVEYAGLVKFDFLGLRTLTIINWALEMINKRRAKNGEPPLDIAAIPLDDKKSFDMLQRSETTAVFQLESRGMKDLIKRLQPDCFEDMIALVALFRPGPLQSGMVDNFIDRKHGREEISYPDVQWQHESLKPVLEPTYGIILYQEQVMQIAQVLSGYTLGGADMLRRAMGKKKPEEMAKQRSVFAEGAEKNGINAELAMKIFDLVEKFAGYGFNKSHSAAYALVSYQTLWLKAHYPAEFMAAVMTADMDNTEKVVGLVDECWRMGLKILPPDINSGLYHFHVNDDGEIVYGIGAIKGVGEGPIEAIIEARNKGGYFRELFDLCARTDTKKLNRRVLEKLIMSGAFDRLGPHRAALMNSLGDALKAADQHAKAEAIGQADMFGVLAEEPEQIEQSYASCQPWPEQVVLDGERETLGLYLTGHPINQYLKEIERYVGGVRLKDMHPTERGKVITAAGLVVAARVMVTKRGNRIGICTLDDRSGRLEVMLFTDALDKYQQLLEKDRILIVSGQVSFDDFSGGLKMTAREVMDIDEAREKYARGLAISLTDRQIDDQLLNRLRQSLEPHRSGTIPVHLYYQRADARARLRFGATWRVSPSDRLLNDLRGLIGSEQVELEFD

SEQ ID NO: 90 - protein, 1221 aa, Thermus thermophilus
MGRKLRFAHLHQHTQFSLLDGAAKLSDLLKWVKETTPEDPALAMTDHGNLFGAVEFYKKATEMGIKPILGYEAYVAAESRFDRKRGKGLDGGYFHLTLLAKDFTGYQNLVRLASRAYLEGFYEKPRIDREILREHAEGLIALSGCLGAEIPQFILQDRLDLAEARLNEYLSIFKDRFFIEIQNHGLPEQKKVNEVLKEFARKYGLGMVATNDGHYVRKEDARAHEVLLAIQSKSTLDDPGRWRFPCDEFYVKTPEEMRAMFPEEEWGDEPFDNTVEIARMCNVELPIGDKMVYRIPRFPLPARRTEAQYLMELTFKGLLRRYPDRITEGFYREVFRLLGKLPPHGDGEALAEALAQVEREAWERLMKSLPPLAGVKEWTAEAIFHRALYELSVIERMGFPGYFLIVQDYINWARRNGVSVGPGRGSAAGSLVAYAVGITNIDPLRFGLLFERFLNPERVSMPDIDTDFSDRERDRVIQYVRERYGEDKVAQIGTLGSLASKAALKDVARVYGIPHKKAEELAKLIPVQFGKPKPLQEAIQVVPELRAEMEKDPKVREVLEVAMRLEGLNRHASVHAAGVVIAAEPLTDLVPLMRDQEGRPVTQYDMGAVEALGLLKMDFLGLRTLTFLDEVKRIVKASQGVELDYDALPLDDPKTFALLSRGETKGVFQLESGGMTATLRGLKPRRFEDLIAILSLYRPGPMEHIPTYIRRHHGLEPVSYSEFPHAEKYLKPILDETYGIPVYQEQIMQIASAVAGYSLGEADLLRRAMGKKKLEEMQKHRERFVQGAKERGVPEEEANRLFDMLEAFANYGFNKSHAAAYSLLSYQTAYVKAHYPVEFMAALLSVERHDSDKVAEYIRDARAMGIEVLPPDVNRSGFDFLVQGRQILFGLSAVKNVGEAAAEAILRERERGGPYRSLGDFLKRLDEKVLNKRTLESLIKAGALDGFGERARLLASLEGLLRWAAETREKARSGMMGLFSEVEEPPLAEAAPLDEITRLRYEKEALGIYVSGHPILRYPGLRETATCTLEELPHLARDLPPRSRVLLAGMVEEVVRKPTKSGGMMARFVLSDETGALEAVAFGRAYDQVSPRLKEDTPVLVLAEVEREEGGVRVLAQAVWTYEELEQVPRALEVEVEASLLDDRGVAHLKSLLDEHAGTLPLYVRVQGAFGEALLALREVRVGEEALAALEAEGFRAYLLPDREVLLQGGQAGEAQEAVPF

SEQ ID NO: 91 - protein, 187 aa, Thermotoga maritima
PEWFIESCKRIKYLFPKAHAVAYVSMAFRIAYFKVHYPLQFYAAYFTIKGDQFDPVLVLRGKEAIKRRLRELKAMPAKDAQKKNEVSVLEVALEMILRGFSFLPPDIFKSDAKKFLIEGNSLRIPFNKLPGLGDSVAESIIRAREEKPFTSVEDLMKRTKVNKNHIELMKSLGVLGDLPETEQFTLF

SEQ ID NO: 92 - protein, 347 aa, Thermus thermophilus
RSGFDFLVQGRQILFGLSAVKNVGEAAAEAILRERERGGPYRSLGDFLKRLDEKVLNKRTLESLIKAGALDGFGERARLLASLEGLLRWAAETREKARSGMMGLFSEVEEPPLAEAAPLDEITRLRYEKEALGIYVSGHPILRYPGLRETATCTLEELPHLARDLPPRSRVLLAGMVEEVVRKPTKSGGMMARFVLSDETGALEAVAFGRAYDQVSPRLKEDTPVLVLAEVEREEGGVRVLAQAVWTYEELEQVPRALEVEVEASLLDDRGVAHLKSLLDEHAGTLPLYVRVQGAFGEALLALREVRVGEEALAALEAEGFRAYLLPDREVLLQGGQAGEAQEAVPF

SEQ ID NO: 93 - protein, 347 aa, Thermus thermophilus
RSGFDFLVQGRQILFGLSAVKNVGEAAAEAILRERERGGPYRSLGDFLKRLDEKVLNKRTLESLIKAGALDGFGERARLLASLEGLLRWAAETREKARSGLLGLFSEVEEPPLAEAAPLDEITRLRYEKEALGIYVSGHPILRYPGLRETATCTLEELPHLARDLPPRSRVLLAGMVEEVVRKPTKSGGMMARFVLSDETGALEAVAFGRAYDQVSPRLKEDTPVLVLAEVEREEGGVRVLAQAVWTYEELEQVPRALEVEVEASLLDDRGVAHLKSLLDEHAGTLPLYVRVQGAFGEALLALREVRVGEEALAALEAEGFRAYLLPDREVLLQGGQAGEVWQPLLF